(19) United States
(12) Patent Application Publication
(10) Pub. No.: US 2005/0287556 A1
(43) Pub. Date: Dec. 29, 2005

(54) Use of HLA-G genotyping in immune-mediated conditions

(76) Inventors: Carole Ober, Chicago, IL (US); Dan Nicolae, Chicago, IL (US)

Correspondence Address: BARNES & THORNBURG, LLP, P.O. BOX 2786, CHICAGO, IL, 60690-2786, US

(21) Appl. No.: 11/123,788

(22) Filed: May 6, 2005

Related U.S. Application Data
(60) Provisional application No. 60/669,773, filed on Apr. 7, 2005.
(60) Provisional application No. 60/568,597, filed on May 6, 2004.

(51) Int. Cl. C12Q 1/68

(52) U.S. Cl. 435/6

(57) ABSTRACT

Assays, including those available in kits, are used to obtain HLA-G genotypes. Kits provide reagents and devices to type biological samples for HLA-G through use of SNPs. Genotypes are predictors of risk.

This application claims priority to two U.S. provisional application 60/669,773 filed on Apr. 7, 2005, and 60/568,597 filed on May 6, 2004.

Methods and compositions for HLA-G genotyping including use of kits, are important additions to current testing for other HLA gener. Genotypes provide predictions of risks for various immune-mediated conditions.

### BACKGROUND

The HLA region on the short arm of chromosome 6 (6p21.3) contains the most polymorphic coding sequences in the human genome.

HLA allele frequency distributions as well as patterns of linkage disequilibrium differ significantly among different human populations. HLA population data can be valuable in estimating the likelihood of finding an HLA-matched donor for bone marrow transplantation, and useful as predictive markers for conditions and diseases such as autoimmune disorders, infertility and allergy.

HLA-G is a special HLA and has been implicated in various immune-mediated diseases and conditions. Specific variants have been reported as associated with risks of miscarriage, and preeclampsia. HLA typing is critical for matching donor and recipients for bone marrow transplantation; the use of well-matched donors increases survival and decreases graft vs. host disease. HLA typing is also important for solid organ transplantation, as well as other areas of use.

Unraveling a highly complex HLA type to a molecular level has many challenges. Many molecular typing platforms have been initially tested on HLA genes to determine their potential performance. A successful typing platform involves expertise in probe and primer design in combination with suitable software to interpret the data to achieve correct results.

High resolution DNA-based HLA typing of population samples of the polymorphic class I loci, HLA-A, -B and -C has only recently become feasible.

HLA-G is a novel HLA gene that has limited polymorphism in the coding region and a restricted tissue distribution. The HLA-G gene was first identified in 1987 but it was not discovered until 1990 that this gene encoded the unusual HLA molecule that was present in fetal placental cells at the maternal-fetal interface. It was subsequently shown that this one gene encodes at least 7 different protein isoforms in the placenta, but specific functions of each and the natural history of their expression pattern during pregnancy are still largely unknown. Functionally, HLA-G is thought to play a central role in the maintenance of maternal immunologic tolerance to the genetically foreign fetus. It has been shown to specifically inhibit or activate maternal natural killer (NK) cells and to inhibit maternal cytotoxic T cells. Thus, it appears that a major function of HLA-G in pregnancy is to inhibit maternal immune responses against genetically foreign fetal cells and to promote the protective T-helper (Th) 2 cytokine milieu that characterizes normal pregnancy.

Recently, it has been demonstrated that HLA-G is expressed in adult macrophages and dendritic cells in response to inflammation and in malignant and non-malignant lung diseases. Further, expression of HLA-G in biopsied myocardic cells from transplanted hearts correlated with prolonged graft survival and transplantation success. In this context, HLA-G is thought to inhibit Th1-mediated inflammation.

The HLA-G gene has limited variation (polymorphism) in its coding region, particularly compared with the other HLA genes, which are the most polymorphic in the human genome. Only 3 polymorphisms cause amino acid changes in the protein sequence and none have been shown to affect protein function. In contrast, a polymorphic variant (called 1597ΔC) is a frameshift mutation and chromosomes carrying this variant do not make any of the two most abundant isoforms, called G1 and G5. This variant is present and in high frequencies in people of African ancestry and is a null for the G1 and G5 isoforms. The 1597ΔC variant has been shown to be associated with recurrent miscarriage. Because of the relative paucity of variation in the HLA-G gene, few groups worldwide study the genetics of this unusual HLA gene.

HLA-G and Reproduction

Approximately 15% of recognized pregnancies end in miscarriage, making it the most common disorder in pregnancy. Moreover, 2-3% of married couples experience 3 or more miscarriages and often do not have any successful pregnancies. The underlying causes for multiple miscarriages are unexplained in half of these couples. Approximately 10% of married couples are infertile (unable to achieve pregnancy), and in about half of couples it is also unexplained. Preeclampsia is a devastating condition that occurs in 5-8% of all pregnancies and is associated with increased maternal and fetal morbidity and mortality. The causes of preeclampsia also remain unknown. The pathophysiology of all three conditions may be related to defects in implantation in some cases. Recently, HLA-G has been implicated in all three conditions further supporting the notion of a common etiology. For example, reduced levels of HLA-G mRNA and protein have been associated with implantation failure after in vitro fertilization (IVF) and with preeclampsia. Polymorphisms in the HLA-G gene have also been associated with miscarriage and preeclampsia, suggesting that polymorphisms in this gene influence expression levels and pregnancy outcome.

HLA-G and Asthma

Asthma affects nearly 14 million people worldwide and has been steadily increasing in frequency for the past 50 years. Although environmental factors clearly influence the onset, progression, and severity of this disease, family and twin studies indicate that genetic variation also influences susceptibility. Linkage of asthma and related phenotypes to chromosome 6p21 has been reported in seven genome screens, making it the most replicated region of the genome. Recently, HLA-G has been identified as an asthma and bronchial hyperresponsiveness (BHR) susceptibility gene on 6p21 and the expression of a soluble isoform (G5) was present in bronchial epithelial cells, expanding this gene's role to include an immune mediated condition.

HLA-G and Adult Cells

Within the past few years, the expression of HLA-G protein in adult cells has been implicated in a number of immune-mediated conditions that involve inflammation or tolerance. For example, HLA-G protein was present in bronchial epithelial cells in the asthmatic lung, in muscle fibers from patients with inflammatory myopathies, in T cell infiltrates in skin cells from patients with atopic dermatitis, and in intestinal epithelial cells of patients with ulcerative colitis. Its immunomodulatory role is also seen in patients undergoing transplants, where detection of HLA-G in the circulation and in the transplanted organ is correlated with increased success rates; moreover, tumors that express HLA-G are more likely to escape immune surveillance by the host.

These combined observations indicate that expression levels of HLA-G are important immune modulators in pregnancy-associated conditions, inflammatory diseases, tumor progression, and transplantation success. Polymorphisms in the promoter region of HLA-G influence transcription levels and, therefore, may underlie differential susceptibility to many common conditions.

### SUMMARY

A method of genotyping a biological sample for HLA-G includes the steps of:
(a) identifying a plurality of Single Nucleotide Polymorphisms (SNPs) in the HLA-G region; and
(b) translating information on the SNPs into a genotype. The plurality of SNPs are identified concurrently or simultaneously in both the promoter and exon regions, for example, in a single amplification reaction, and in combination are a genotype.

The plurality of SNPs are located either in the promoter region of HLA-G or in both the promoter and in the exons of HLA-G or in either region.

SNPs in the promoter region are listed in Table 7 and are identified by, e.g. direct sequencing.

SNPs in exons are identified, for example, by hybridization with specific probes.

SNPs in exons and promoter are listed in Table 8 as alleles (haplotypes) combined into genotypes.

The promoter region of HLA-G includes a DNA sequence from nucleotide position about 1350 to about +50 of the HLA-G promoter.

In an embodiment, the HLA-G promoter region is amplified by primers selected from the group consisting of nucleotide sequences designated 5′-AACAGTGCTAGAGCCACAG), 5′-AACAGTGCTAGAGCCACAA, 5′-GAAGAGGGTTCGGGGC, and 5′-GAAGAGGGTTCGGGGT.

A genotype is defined as a string of pseudo codes consisting of 0, 1 and 2 wherein the string of pseudo codes is selected from the string of pseudo codes listed in Table 8 by use of algorithms described herein.

A method for predicting an immune-associated risk in a subject includes the steps of:
(a) obtaining an HLA-G genotype of the subject and
(b) determining the immune-related risk of the subject by comparing the subject's HLA-G genotype as determined by methods and compositions described herein, with a reference HLA-G genotype. The difference may be indicated by levels of circulating HLA-G.

Immune-associated risks are determined, for example, for asthma, allergy, auto-immune disorders, infertility, fetal miscarriage, and tissue or organ transplantation. Risks may be determinded by expressed levels of HLA-G. Immune-associated risks may be is determined for a Th2-skewed immunologic condition.

An assay for a candidate drug to ameliorate a symptom of an immunologic condition, includes the steps of:
(a) determining association of on HLA-G genotype expression with the immunologic condition; and
(b) determining if the candidate drug affects the expression of the associated HLA-G.

A method for ameliorating the effects of an immunologic condition includes the steps of:
(a) determining an HLA-G polymorphism that is associated with the condition;
(b) determining an effect of the polymorphism on a metabolic pathway; and
(c) modulating the effect on the metabolic pathway to ameliorate the immunologic condition.

An HLA-G asthma susceptibility gene is useful to develop an inhibitor of an asthma symptom in a person at risk. Inhibitors may be delivered by an inhaler.

A diagnostic kit for HLA-G associated immunologic conditions includes:
(a) primers capable of amplifying HLA-G; and
(b) reagents to amplify HLA-G and identify SNPs. Primers specific for promoter haplotypes and reagents for sequencing to detect SNPs are in the kits. Primers specific for all SNP polymorphisms, in the promoter and exons, and reagents for hybridization to detect SNPs, are in the kits.

Further, information on HLA-G genotype associated risks for immunologic conditions are in the kits.

### BRIEF DESCRIPTION OF THE DRAWINGS

FIG. 1 is a schematic of the 5′-upstream regulatory region of HLA-G and the location of 27 polymorphisms. “+1” designates the transcriptional start site. Cis-acting regulatory elements are shown: TATA=TATA box; CCAAT=CAAT box; S/X1=Pan HLA regulatory elements; ISRE=interferon-specific regulatory element; EnhA=enhancer A; HSE=heat shock protein element; GAS=gamma (interferon) activated site; LCR=locus control region (also known as “TSRE” [tissue-specific regulatory element]). The LCR (or TSRE) contains the regulatory elements that direct trophoblast-specific expression (Schmidt and Orr 1995).

FIG. 2 (A-H) shows the full sequences of the 14 haplotypes defined by 27 polymorphisms in the 5′-upstream regulatory region of HLA-G.

FIG. 3 shows that in vitro expression assays reveal significant differences between HLA-G haplotypes; similar results were obtained in HBE cells. Promoter-specific expression levels a) (FIG. 10) at 24 and 40 hours of culture (baseline); b) cultured in 0 and 100 ng IL-10; c) cultured in 0, 10, 100, and 500 ng/ml TRAIL; d) in cells grown in 18% (normal culture conditions) and 2% O₂; and e) following heat shock (cell stress).

FIG. 4 shows EMSA using nuclear extract from cultured JEG-3 cells. Labeled oligos corresponding to the −725C/G and −716T/G polymorphisms were included in each lane along with the nuclear extract, as indicated above each lane. Cold competitor oligos were included in lanes 3-7,9-12, and 14-18, as indicated under each lane. Lanes 1, 7, and 13 did not include labeled or competitor oligos; lanes 2, 8, and 14 contained nuclear extract and labeled oligos, but no competitor oligo. Lanes 1-12 were from a single gel; lanes 13-18 were from a second gel.

FIG. 5 shows that the mAB 1-2C3 detects soluble HLA-G5 in HBE cells. Solid arrows show bronchial epithelial cells; dashed arrows show bronchial mucosal glands. All shown at 10×. Panel A: asthmatic lung stained with antibody 1-2C3 (anti-G5); Panel B: non-asthmatic lung stained with antibody 1-2C3; Panel C: asthmatic lung stained with IgG (negative) control; Panel D: non-asthmatic lung stained with IgG (negative) control). Antibodies specific for HLA-G1, -G2, and -G6 were negative in both asthmatic and non-asthmatic lung sections.

FIG. 6 shows haplotypes comprised of 23 polymorphisms in the 5′-upstream regulatory region, in exons 1 and 8, and their associated HLA-G allele. Frequencies of haplotypes in the Hutterites are shown. Alleles shown as black letters on a white background correspond to the chimp sequence and are presumed to be ancestral; alleles shown as white letters on a black background are presumed to be derived alleles. Alleles at two polymorphic sites in exon 1 are shown for comparison with earlier studies (Hviid et al. 1999).

FIG. 7 shows sequence flanking the −725C/G polymorphism in bisulfite-treated DNA. A, Genotype −725 CC. B, Genotype −725 GG.

FIG. 8 shows linkage to 6p21 in the Chicago families. A) The dashed line shows the results in the initial genome screen with framework markers. The solid line shows the results with five additional STRPs between framework markers D6S1281 and D6S258; B) 1 Mb region from D6S258 to D6S265 with positional candidate genes (all known genes, but not all pseudogenes (oval enclosure) and STRPs (rectangle enclosure), are included). Circles=SNPs; triangles=in/dels; squares=STRPs; HLA-A genotype comprised of multiple SNPs shown as a rectangle. Results of association studies with each polymorphism in four populations are shown in Table 6.

FIG. 9 shows LD block structure in the extended class I region. A) Graph of LD map (Zhang et al., 2002). Shaded boxes show five blocks in this region. B) Pairwise TDT of variants within each block. Block 2 is not shown because the two SNPs in the block were genotyped only in the families and there was no evidence of nonrandom transmission (P>0.05). Results in Chicago families are shown in the lower half and trios in the upper half. P-values were derived by simulations that conditioned on the evidence for linkage, but were not corrected for multiple comparisons.

FIG. 10 shows in vitro expression assays revealed significant differences between HLA-G haplotypes in a human bronchial epithelial (HBE) cell line. Results from nine independent experiments are shown. The G*01011 allele is the most common HLA-G allele and carries −964G. The G*01012 allele is the second most common HLA-G allele and carries the −964A allele. Two additional haplotypes were identical to the G*01011 haplotype except that one has a G at −725 (the other alleles shown have a C at this site), and one has both a G at −725 and a T at −1121 (all other alleles shown have a C at −1121). Expression levels were significantly different among the different haplotypes (ANOVA, P=0.005).

FIG. 11 shows haplotype-specific PCR of the HLA-G promoter region, demonstrating the specificity of the assay. DNA in lanes 2-5 were amplified with primers for −1306G and +15G, DNA in lanes 6-9 with primers for −1306A and +15A, DNA in lanes 11-14 with primers for −1306G and +15A, and DNA in lanes 15-18 with primers for −1306A and +15G. PCR reactions were performed in 50 μl reaction with 25 ng template DNA, 5 μl 10× PCR buffer without Mg, 2 μl 2.5 mM dNTP mix, 1 μl forward primer (10 μM), 1 μl reverse primer (10 μM), 3 μl 25 mM MgCl₂, 0.5 μl DMSO, 8 μl 50% glycerol, 0.2 μl Platinum Taq polymerase (5U/μl), and 28.8 μl sterile H₂O. PCR conditions were 95° C. for 10′; 40 cycles at 94° C. for 45′, 60° C. for 1′, and 72° C. for 2′; final cycle at 72° C. for 7′; holding cycle at 4° C. The haplotype (GA) or diplotypes (GG/GG, AA/AA, AG/AG) of the DNA in each lane is shown under the gel image. DNA ladder is in lanes 1, 10, and 19. Because the G-A or A-G haplotype was not observed in any human sample, chimpanzee DNA was used for the A-G control in lanes 5, 9, 14, and 18 and constructed a G-A haplotype in the lab (lanes 4, 8, 13, 17).

FIG. 12 Results of luciferase assays with HLA-G promoter haplotypes in JEG-3 transfected cells (cultured for 40 hours). Transfections were performed in triplicate; the mean renilla-normalized luciferase activities for 9 independent experiments are shown. Promoters were compared using 2-way ANOVA. Overall, there were significant differences in expression levels between the different promoters (P<0.001). The promoters with −725G (dark gray bars) had higher expression levels than promoters with a −725C (asterisk bars) (P<0.001) or a −725T (light gray bars) (P<0.001). There were no differences between the promoters carrying a −725C and the promoter carrying a −751T (P=0.204). These results indicated that 1) different promoters are associated with different expression levels, and 2) the −725G allele is associated with increased transcription in JEG-3 cells.

FIG. 13 The −725G allele is associated with increased transcription (results based on 13 experiments, each performed in triplicate). To further explore the differences observed in the above experiments, site-directed mutagenesis is used to determine whether the −725G allele and/or the −1121T allele is responsible for the greater transcriptional activities of these promoters and if they require being on a G*01011 background. To differentiate between these possibilities, a G at −725 on the *01012 promoter (last bar on right in FIG. 6) is inserted and a C at −725 on the *01011c (next to last bar in FIG. 6) promoter. Neither of these promoter haplotypes has been observed in the Hutterites or in >400 EA and AA control and asthmatic chromosomes. Among the naturally occurring haplotypes (asterisk), there were differences in expression levels between haplotypes (P<0.0001), as in FIGS. 3, 10. Promoters with the −725G allele (G*0101 lb) or the −725G/−1121T alleles (G*01011c) show higher expression levels than G*01011a and G*01012. Expression levels of the mutated G*01012 haplotype (−725C-G) were increasing compared with the derivative haplotype (2-way ANOVA, P<0.001) and decreased expression of the mutated G*01011c haplotype (−725G-C) compared with their derivative haplotypes (2-way ANOVA, P<0.001). However, the expression levels of the mutated G*01011c haplotype was intermediate between the G*01001c and haplotypes carrying −725C and the mutated G*01012 haplotype was intermediate between G*01012 and haplotypes with −725G, suggesting that additional variation on the G*01011 and G*01012 haplotypes influence expression levels.

### DETAILED DESCRIPTION

Previous HLA typing methodology is both limited and laborious. In fact, there are currently no kits or straightforward protocols available for genotyping the SNPs (single nucleotide polymorphisms) in the HLA-G gene. Although this gene has fewer polymorphisms in its exons (coding region) that the other HLA genes, it is still a challenging gene to genotype or sequence due to the high GC content and high homology with other HLA genes (the latter making specific PCRs challenging). The extraordinary levels of polymorphisms in the promoter region make genotyping this region of the gene particularly difficult. Sequencing genomic DNA from individuals was performed to determine HLA-G genotypes, but because most individuals are heterozygous in this region of the gene, sequencing calls were often ambiguous. In Ober et al., 2003, individual haplotypes were cloned into vectors so that many SNPs could be confirmed and assigned haplotypes (phase) in individuals heterozygous for multiple SNPs. That approach, while highly accurate, is very labor-intensive and is not optimal for genotyping large numbers of individuals.

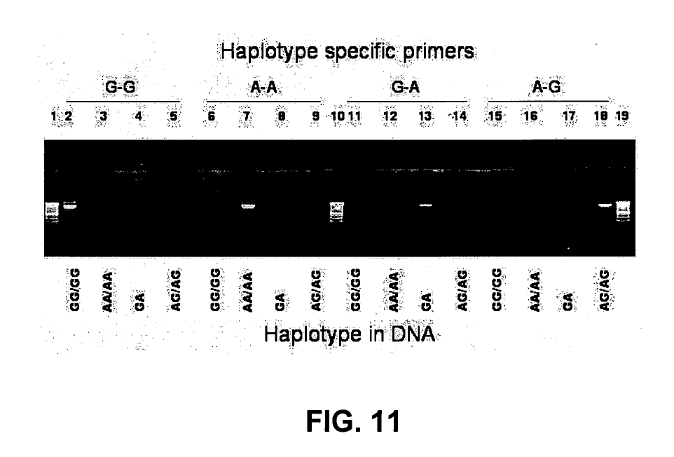

Therefore, a novel method for haplotype-specific PCR followed by direct sequencing (HSP-S) was developed for genotyping polymorphisms in the promoter region of HLA-G. (FIG. 11) 5′-primers were designed that were specific for either the −1306G (5′-AACAGTGCTAGAGCCACAG) or −1306A (5′-AACAGTGCTAGAGCCACAA) allele and 3′-primers that were specific for either the +15G (5′-GAAGAGGGTTCGGGGC) or +15A (5′-GAAGAGGGTTCGGGGT) allele. Each sample was amplified with each of 4 pairs of primers specific for (G-G, A-A, G-A, or A-G) along with control DNA containing each of these 4 combinations. The G-A haplotype was artificially constructed in the laboratory because it does not exist in nature. The Chimp DNA was used as a control for the A-G haplotype. The PCR product(s) and genotypes were sequenced and haplotypes determined for each sample. The sensitivity and specificity of this method was tested in a blinded study using DNA from 8 individuals with known genotypes, selected to represent different combinations of promoter haplotypes. The correct haplotype was identified in each of these 8 samples. This approach was relatively rapid and avoids, for the most part, ambiguous calls in heterozygous sequences. This approach was used to haplotype several hundred individuals and, as a result, 27 promoter SNPs were identified in these samples and the haplotype structure was characterized in European Americans, African Americans, and Asians.

In Table 7, the configuration of 28 SNPs on chromosomes (called halotypes). These SNPs occur in combinations that define 14 unique halotypes, each of which is associated with an HLA allele (column 1). The frequencies of these halotypes in three population samples are shown in columns 2-4. The polymorphic nucleotide positions (relative to the start translation) are shown in the headers of columns 5-32, and the base pair at that position on each halotype shown.

HLA-G polymorphisms may be related to metabolic products. If HLA-G products are associated with a disease or condition, testing can be directed to see if candidate drugs affect levels of the products. HLA-G assays are useful to develop drugs to ameliorite Th2-skewed immunologic conditions.

Risks may be predicted based on HLA-G genotypes. For example, recurrent miscarriage risk may be related to a specific genotype e.g. −725G HLA allele, that affects the amount of HLA-G protein produced.

An asthma susceptible gene may be used to develop inhibitors to acute episodes of asthma e.g. by means of an inhalant. Symptoms of allergy and asthma may be alleviated by administering agents that reduce HLA-G expression.

New Algorithm for Genotyping HLA-G

This is a method for determining HLA-G genotypes in diploid DNA, after genotyping each sample for 41 polymorphisms that define 21 unique alleles (or haplotypes), which includes polymorphisms in the promoter and exons 1, 2, 3, 4 and 8. This method is suitable for use with a genotyping platform where each SNP is typed in genomic DNA. There are a total of 231 possible genotypes that can occur in diploid DNA as combinations of these 21 alleles.

In Table 8, each individual's genotype at the HLA-G locus is composed of two different haplotypes. All possible pairs of haplotypes (called diotypes) are shown in columns 1 and 2. SNPs in the promoter region, exon 1, exon 2, Exon 3, exon, 4 and exon 8 are shown in the headers of columns 3-30. under each of these columns is the number of “wild type” alleles at each site corresponding to each diotype. The sequence of the G*010102/*010102 will have a score of “2” at every polymorphic site. Each diotype has a unique “string” of 43 scores, which can be determined by genotyping.

To determine the genotype, each sample is scored at each polymorphic site as 0, 1, or 2 pseudo codes corresponding to the number of copies of the reference allele at that site, using the G*01012a allele (Table 7) as the reference sequence. For the one tri-allelic SNP at −725, each individual is scored for each of the 3 alleles (C, G, T). Thus, there are a total of 43 scores corresponding to the 41 polymorphic sites. Each genotype will have a unique “string” of 43 scores, which will allow assigning genotypes to each individual. New alleles (comprised of known SNPs, but in different configurations) will also be detected by this method because the “string” will not match any of the known genotype combinations. As new alleles are defined, the genotyping algorithm will be modified to include these new discoveries. The promoter region sequence has not yet been defined for two rare alleles (called G3d5 and 010101 g), but these alleles can still be distinguished from the other alleles using coding region (exons 2 and 3) variation.

Using HLA-G SNPs in The Roche Method

In the method of genotyping HLA-G using methods developed by Roche, each SNP is typed in genomic DNA. DYNAL provides kits for testing for the classical HLA genes (HLA-A, B, Cw, DRV, OBD using the Roche method (Dynal Biotech, Oslo, Norway, www.dynalbiotech.com). It is contemplated that similar methods are suitable for use with HLA-G SNPs (Table 7). For example, Dynal's RELI™ SSO Typing generally involves a generic PCR amplification with biotinylated primers that flank the specific target region of an HLA locus. This amplification is followed by hybridization of the resulting amplicon to an array of sequence specific oligonucleotide (SSO) probes immobilized on a nylon membrane. The immobilization of these probes is desingated as reverse line blot, the acronym RELI™.

Under appropriate wash conditions, the probes remain bound only to their complementary sequence in the amplified DNA. The sensitivity of the system enables single nucleotide differences to be distinguished. The presence of the amplicon/probe complex is detected using a simple calorimetric reaction and appears as a blue precipitate.

RELI™ SSO Pattern Matching Program (PMP) Software provides interpretation of results and data management system. Automated data input-PMP receives strip image directly from Dynal RELI-Scan™ instrument or a flat bed scanner. Each probe has an individual signal intensity cut off value for accurate assignment of positive and negative signals. Software combines data from multiple tests to produce a single result. Software recommends typing kit solutions to resolve ambiguities. PMP allele Database is regularly updated to ensure all new alleles are included. Updates can be downloaded from Dynal Biotech website or e-mailed directly. Typing kits, instructions for interpreting data tables and methods can be obtained from Dynal Biotech.

The HLA-G gene is primarily expressed in placental cells that invade the maternal decidua during pregnancy. This gene encodes multiple isoforms that fulfill a variety of functions at the maternal-fetal interface throughout gestation. A null allele for the most abundant HLA-G isoform was associated with recurrent miscarriage in two independent studies, suggesting that reduced levels of the HLA-G1 protein may compromise successful pregnancy. To determine whether other polymorphisms that could affect expression levels of HLA-G were associated with fetal loss in women participating in a 15-year prospective study of pregnancy outcome. These were genotyped subjects for 18 single-nucleotide polymorphisms in the 1,300 bp upstream of exon 1, as well as for an insertion/deletion (in/del) polymorphism in the 3′ untranslated region. The 18 SNPs defined eight unique haplotypes. One polymorphism, −725C/G, was associated with fetal loss, with an increased risk for miscarriage in couples in which both partners carried the −725G allele, compared with couples not carrying this allele (odds ratio 2.76, 95% confidence interval 1.08-7.09;) P<0.035. Further, the G at nucleotide −725 creates a CpG dinucleotide, this CpG site is methylated on −725G alleles. Overall, extraordinary levels of variation were identified in the 5′-upstream regulatory region of HLA-G and provided evidence for an association between a promoter-region SNP and fetal loss rates, further attesting to the novel features and critical role of this gene in pregnancy.

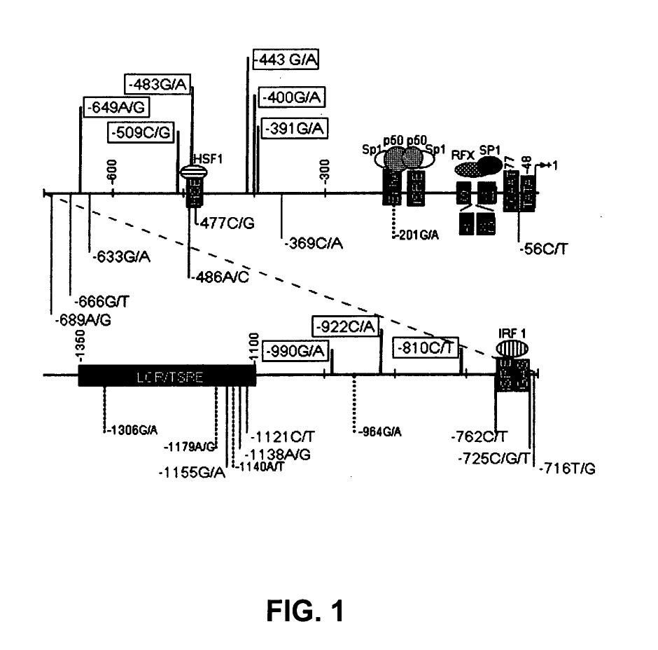

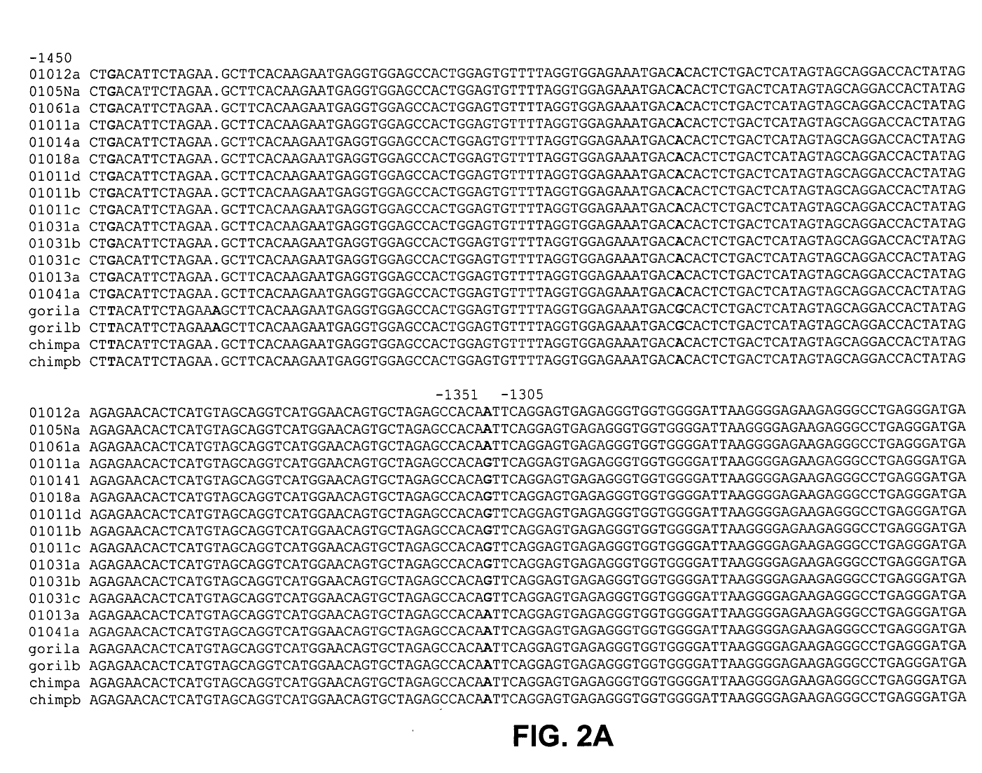

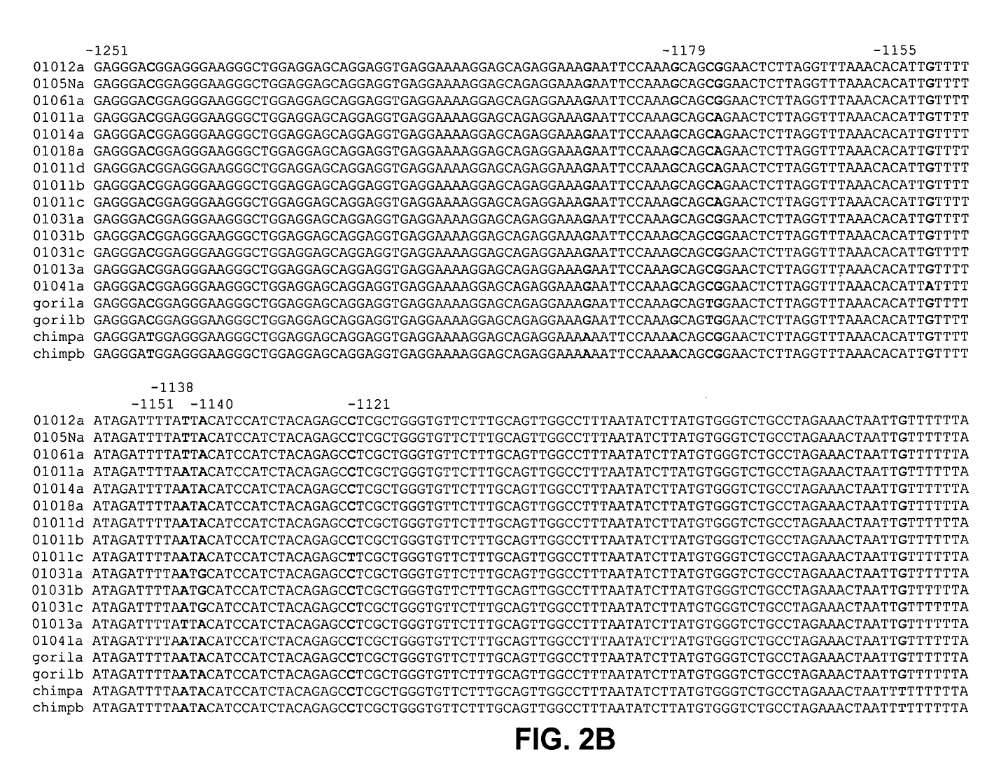

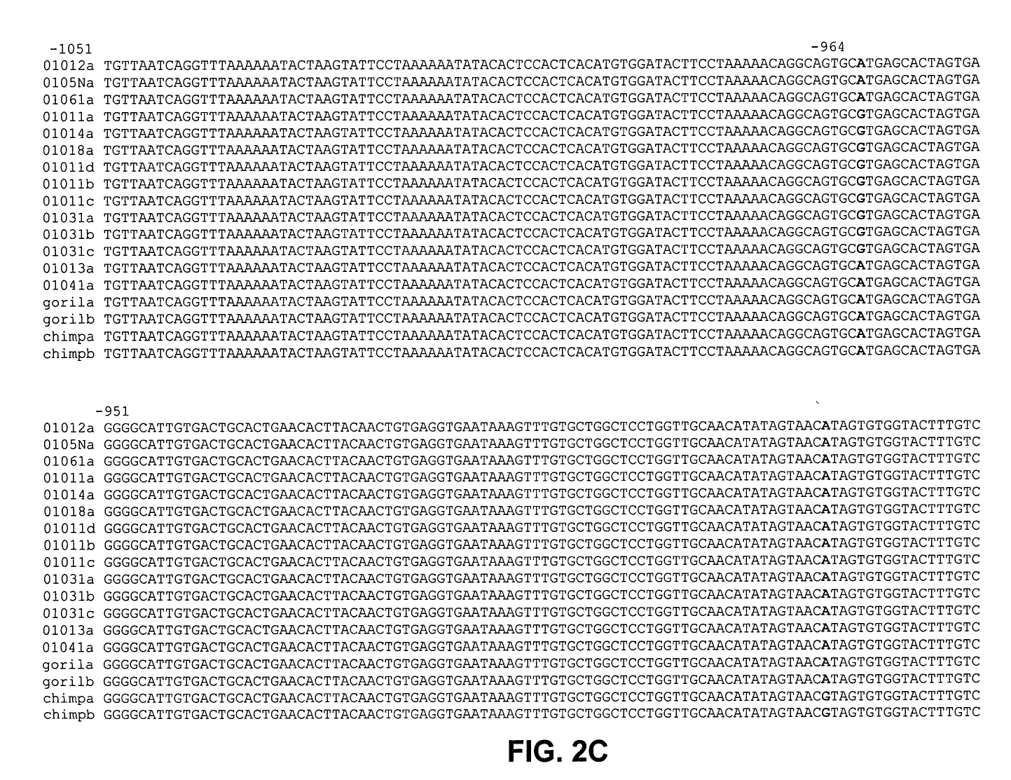

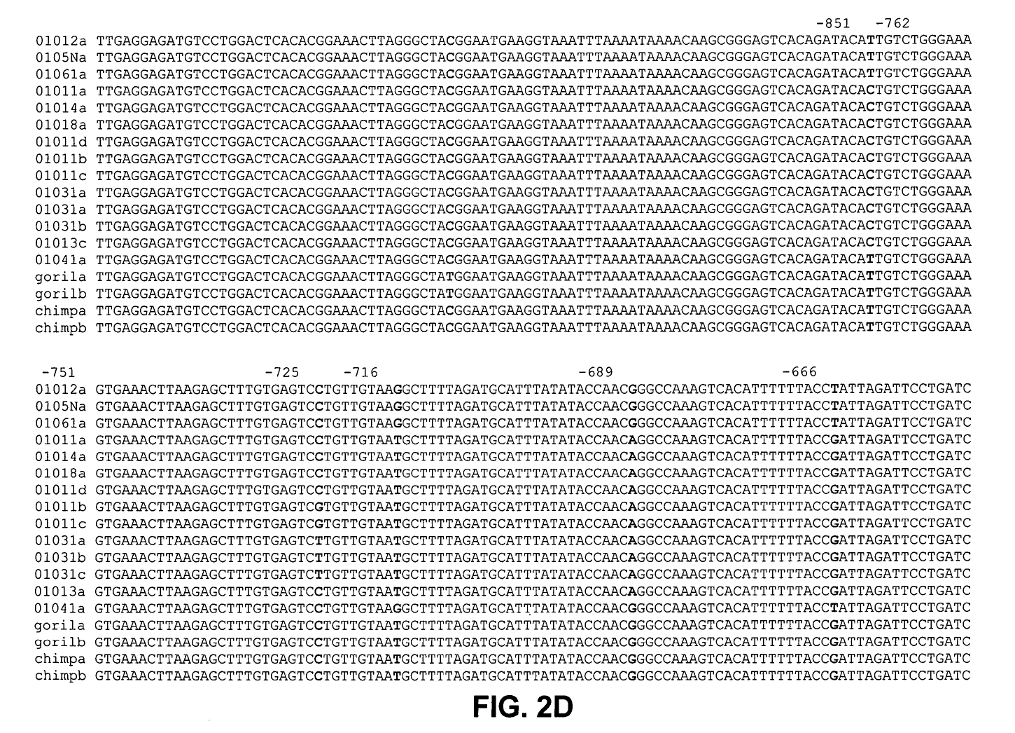

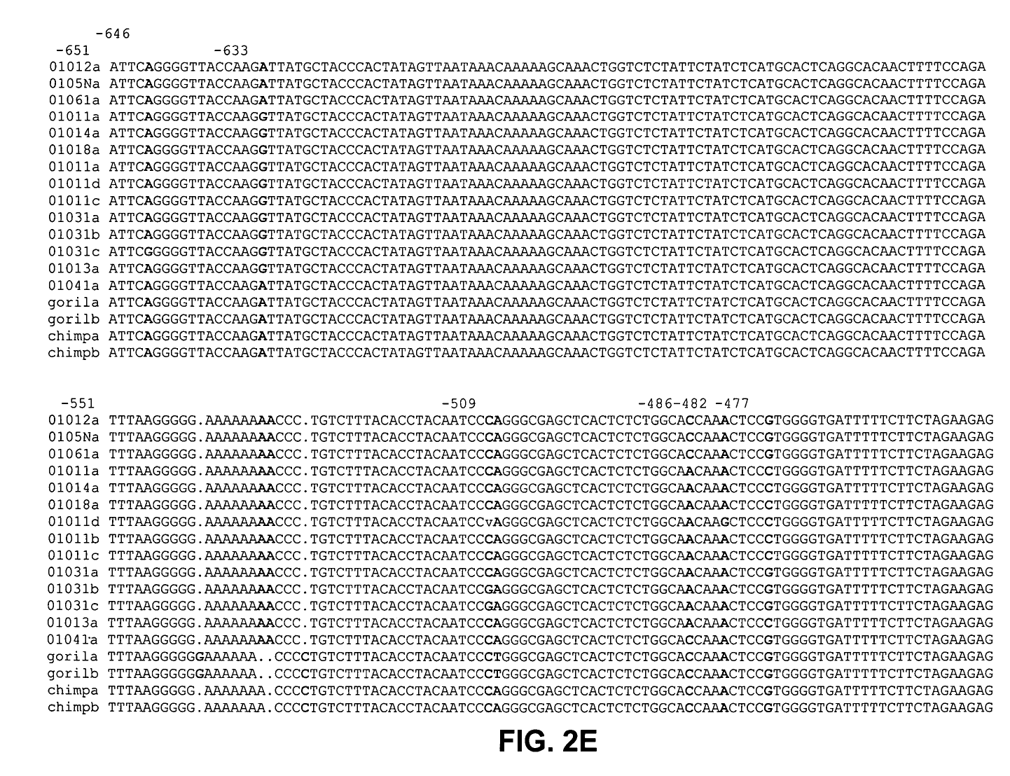

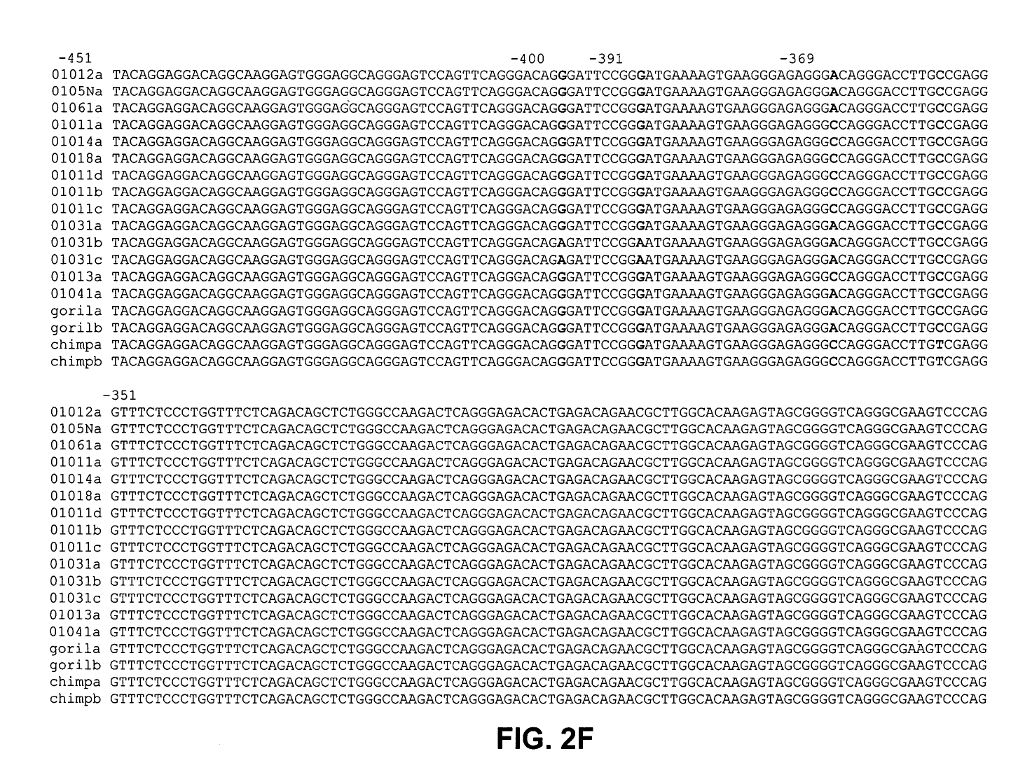

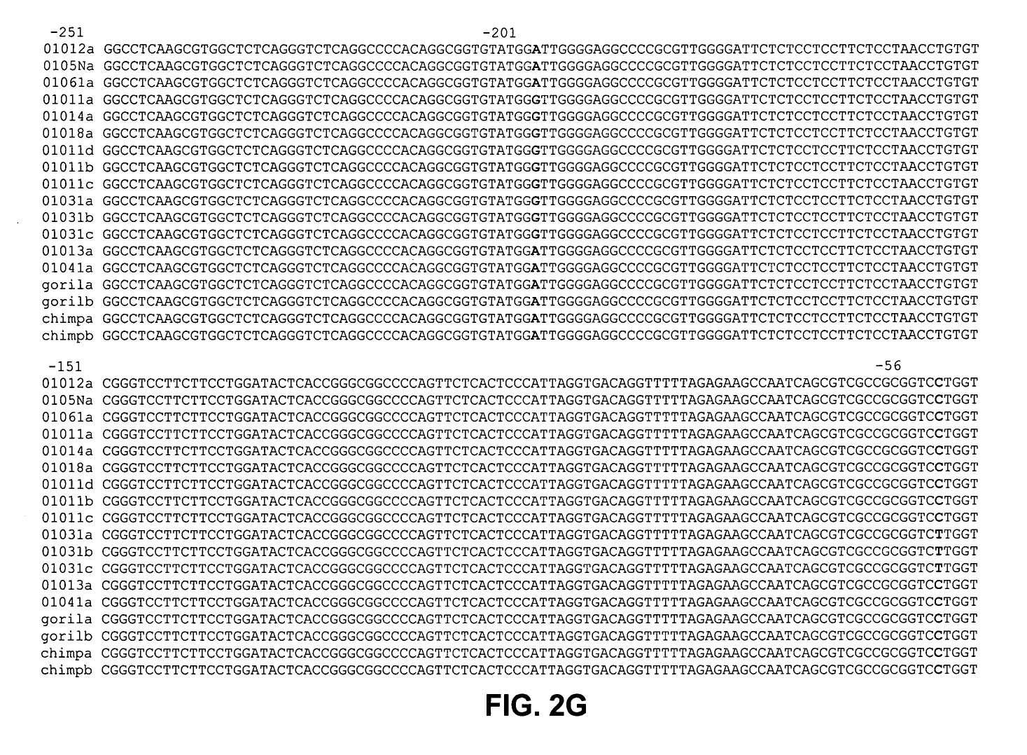

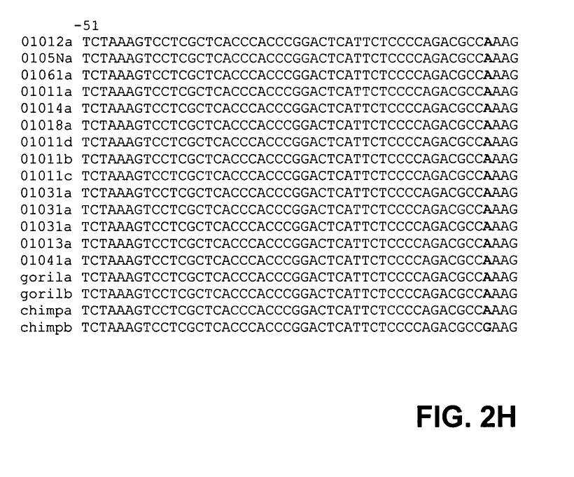

The association between the 1597ΔC null variant and recurrent miscarriage suggested that reduced levels of the G1 and/or G5 proteins are a risk factor for pregnancy loss. Therefore, to see if protein expression levels influence the immunologic milieu in pregnancy (and pregnancy outcome), additional variation was sought in the ˜1500 base pairs (bp) upstream from exon 1, a region that contains all of the known elements that regulate the transcription of HLA-G messenger (m) RNA. These studies led to the discovery of 17 novel polymorphisms in this important regulatory region, in addition to 5 that had been previously reported. These 22 polymorphisms occur on 10 different chromosomal backgrounds, called haplotypes. The locations of these polymorphisms relative to the known regulatory elements are shown in FIG. 1 and the sequences corresponding to these 10 haplotypes are shown in FIG. 2. One of the variants (−725G) is associated with miscarriage. The C→G change at position −725 resulted in a change in the methylation pattern at this site, and suggested that differential methylation could influence the regulation of transcription in HLA-G, as it does in other genes.

Based on genetic linkage and association studies and functional studies, the HLA-G gene is identified as an asthma susceptibility locus. The discovery opens up avenues for novel therapeutic strategies for asthma allergy, as well as for other immune medicated diseases, that have been linked or assciation to the HLA region on chromosome 6, such as multiple sclerosis, psoriasis, atopic dermatitis, inflammatory bowel disease, schizophrenia and most autoimmune diseases.

In this disclosure evidence is presented from studies in four independent samples for a novel asthma and bronchial hyperresponsiveness (BHR) susceptibility locus in the human leukocyte antigen (HLA) region on chromosome 6p21. This locus may contribute to risk for other inflammatory diseases that show linkage to this region.

Functional studies demonstrated that variation in the upstream region of HLA-G influences transcription rates in an in vitro assay. The role of variation in this gene in asthma and allergic disease, another group of “conditions” that are characterized by a skewed Th2 cytokine profile were investigated. Expression (or overexpression) of HLA-G in the lung may skew the immune response to airborne allergens in the lung in a Th2 direction, thereby either initiating or facilitating the characteristic Th2-mediated inflammatory response in the asthmatic lung. Molecular evolutionary studies of the upstream region of HLA-G showed that the variation in this region has been influenced by natural selection, indicating a functional role for some or all of these 22 polymorphisms.

### EXAMPLE 1

### In Vitro Studies Show Significant Haplotype-Specific Differences in Transcription Rates and in the Binding of Nuclear Proteins

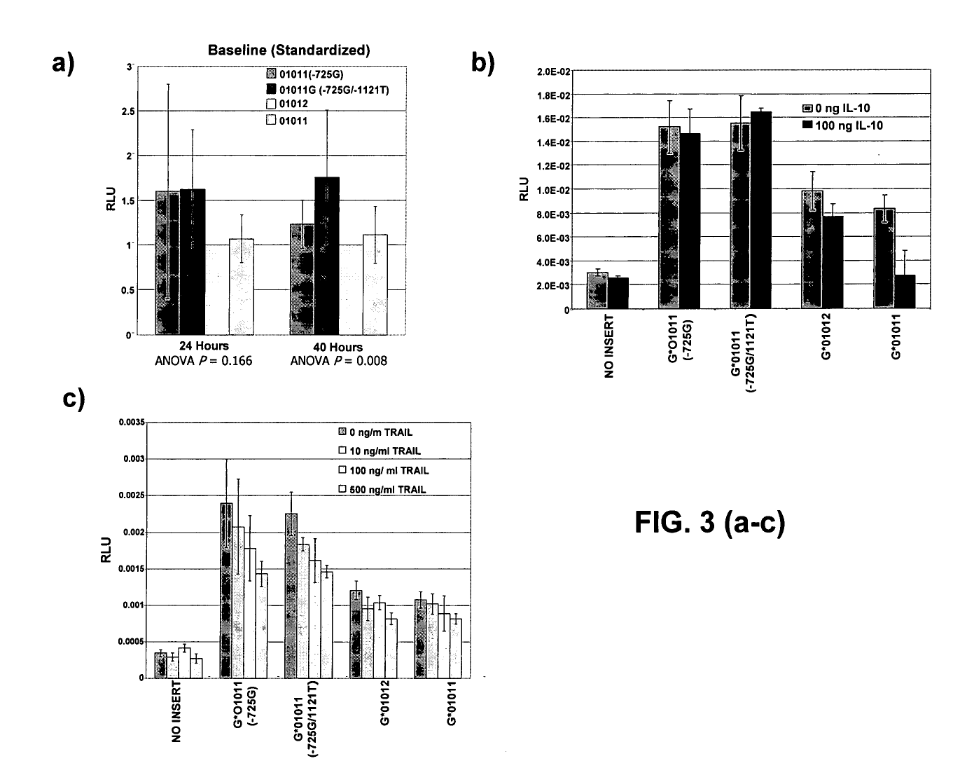

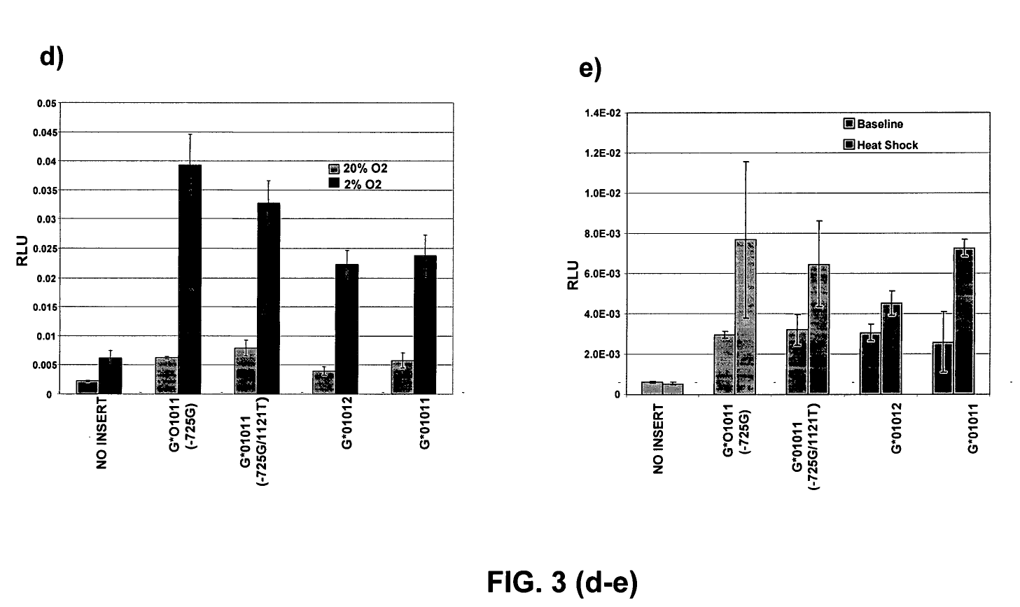

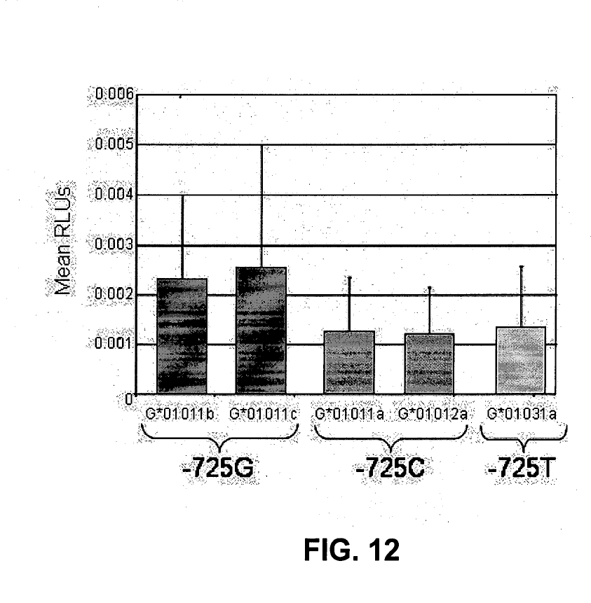

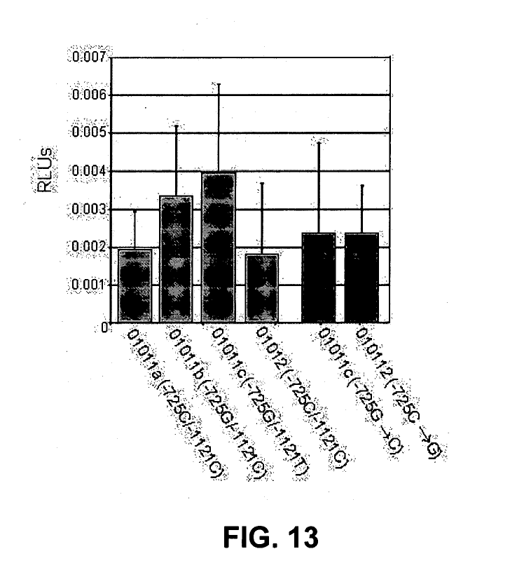

Each of 5 haplotypes disclosed were cloned into an expression vector carrying the firefly luciferase gene, and these vectors were transfected in a trophoblast (placental cell) line (called JEG-3) and a human bronchial epithelial cell line (called HBE). Expression studies were completed for these haplotypes including 2 that carry the miscarriage-associated −725G allele. Activity levels of these promoters were compared at baseline, in response to 9 cytokines that are relevant in both pregnancy and asthma, and under conditions of heat shock (stress) and low oxygen. Under all of the conditions tested to date, the 2 promoters with a G at −725 show significantly higher levels of expression compared with 2 promoters lacking the G at −725. Further, under some culture differences there are differential responses. The results of some representative experiments are shown in FIG. 3 and FIG. 12 and a summary of the experiments in JEG-3 cells are shown in Table 1. Site directed mutagenesis was used (FIG. 13).

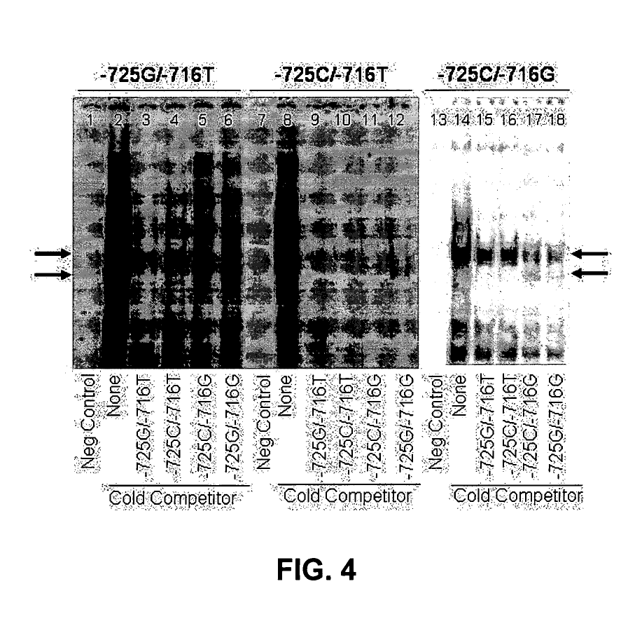

To determine if the miscarriage-associated SNP, −725C/G, influences the binding of nuclear proteins, electrophoretic shift assays (EMSAs) were conducted to determine whether the −725G allele could influence promoter function by affecting binding of nuclear proteins to the neighboring IRF-1 site or to a transcription factor binding site that coincides with this variant. To test the latter, 32-mers were designed that share identical sequence except at the −725C/G or −716T/G sites. Because of the proximity of the −716 SNP, this SNP was included 4 pairs of sense (s) and antisense (as) oligos for annealing and competition in the EMSAs. The oligos were radiolabelled with p³² and incubated with nuclear extracts from cultured JEG-3 cells by themselves and with excess cold oligos (competitors) included each of the 4 sense oligos and the corresponding antisense shown above. The results of these studies are shown in FIG. 4. In the lanes 2, 8, and 14 (without competitors) a number of bands are visible. One band, corresponding to the lower arrow is present in lanes that include oligo containing the −716T allele (lanes 2 and 8), but not in the lane with oligo that contains the −716G allele (lane 14). This indicates that the band corresponding to the lower of the two arrows represents the binding of nuclear proteins to oligos containing the −716T allele. Further, this lower band is competed away in lanes that include cold competitor containing the −716T allele (lanes 3, 4, 9, 10), indicating that these nuclear proteins are specific to the −716T allele. In the lanes that include labeled oligos with the −716G allele, there is no binding of nuclear proteins corresponding to the lower arrow (lanes 14-18). Instead, in these lanes there is band corresponding to the upper arrow, which is also visible in the lanes that contain an excess of cold competitor containing −716T (lanes 3, 4, 9, and 10). Thus, the binding of nuclear proteins at the site of the lower arrow correlates with the presence of the T allele at −716. Oligos with the −716G allele do not specifically bind these nuclear proteins, and the −725C/G polymorphism appears to have no effect on binding. In summary, this experiment indicates that a variant in the promoter region at −725 differentially binds nuclear proteins, which can alter transcription rates of HLA-G mRNA.

### EXAMPLE 2

### Genetic Variation in HLA-G is Associated with Increased Risk for Asthma and Allergy

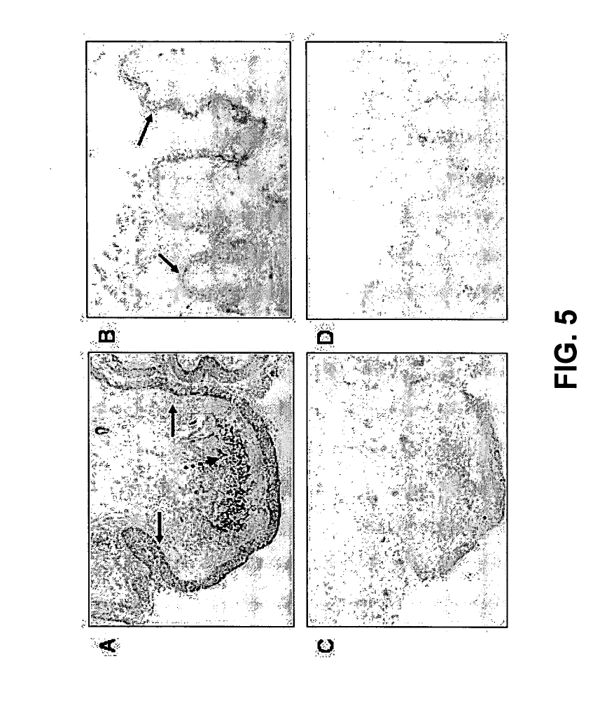

Because both pregnancy and allergic disease (including asthma) are characterized by a Th2-skewed immunologic environment, HLA-G is a candidate for the chromosome 6p21-linked asthma gene. A genetic linkage to this region of chromosome 6 had been reported in 7 genome-wide screens for asthma-susceptibility loci (Ober et al. 2000; Xu et al. 2001; Hoffjan and Ober 2002), By immunohistochemistry it was demonstrated that the G5 isoform of HLA-G is expressed in bronchial epithelial cells (FIG. 5). Variation in HLA-G, including the 5′-upstream regulatory region was surveyed to see if any of this variation could explain the original evidence for linkage to chromosome 6p21 in Chicago area asthma families. Variation in HLA-G accounted for nearly all the evidence for linkage on 6p, whereas none of the variation surveyed (>40 polymorphisms in 19 genes on 6p21) reduced the linkage evidence as much. It is likely that HLA-G is the asthma susceptibility locus on 6p. 3 additional populations were genotyped to see if any of the HLA-G variants were associated with susceptibility to asthma or the closely related phenotype, atopy (or allergy). Variation in HLA-G was associated with asthma or allergy in all 4 populations. In particular, the −964G/A polymorphism in the regulatory region was associated with asthma in the Chicago asthma families (P=0.053) and a second sample of asthmatic children from Chicago (P=0.040), and with bronchial hyperresponsiveness (BHR, a measurement of lung reactivity that defines the asthma phenotype) in members of a population isolate living in South Dakota (P=0.046) and in Dutch children of mothers with asthma (P=0.004). Further, the −964G/A polymorphism was associated with atopy (defined by a positive skin test to airborne allergens) in a Dutch population (P=0.006) and the high expressing −725G allele was associated with atopy in the South Dakota population (P=0.0051). Additional variation in HLA-G was associated with asthma or BHR with P-values as small as 0.013 in the Chicago families and 0.034 with −201G/A in the Chicago replication sample. Associations between variants in HLA-G and asthma or atopy in 4 distinct populations further provide compelling evidence that HLA-G is an asthma susceptibility locus.

An association with the 1597ΔC allele was examined to see if reduced expression of HLA-G5 protein is protective against the development of asthma or atopy. Because the frequency of this variant is very low in Caucasian populations, the frequency among a sample of African Americans with severe asthma was determined. Among these patients, the null allele was significantly less frequent among subjects with atopy compared to those without. Thus, these studies also support a role for HLA-G in asthma and allergy, and further suggest that reduced expression of HLA-G may be protective against allergic disease.

Many other diseases with an inflammatory component have been linked to chromosome 6p21, such as multiple sclerosis, psoriasis (Zhang et al. 2002), atopic dermatitis (Soderhall et al. 2001), inflammatory bowel disease (Mathew and Lewis 2004), and schizophrenia (Wright et al. 2001). To date, the variation underlying these linkages has not been identified. Variation in the HLA-G gene may contribute to susceptibility to a wide range of inflammatory diseases, including asthma, as well as disorders of pregnancy, such as miscarriage.

### EXAMPLE 3

### Variation in the 5-Upstream Region of HLA-G has been Maintained by Natural Selection

The extensive amount of variation in the upstream region of HLA-G, the relatively high frequency of most of the alleles, and the pattern of variants on each haplotype are quite striking. Molecular evolutionary studies were used to determine whether natural selection has patterned and maintained variation in this region. Finding evidence for natural selection would further indicate a functional role for the variation. Preliminary studies of 100 chromosomes from 50 individuals indicate that the variation in this region can not be explained by neutral evolution, but rather that natural selection has been acting on this variation. The amount of variation and the frequency spectrum of alleles are summarized in this region. Nucleotide diversity was 6×10⁻³, considerably higher than the genome average. Further, Tajima's D statistic, which summarizes the frequency spectrum, was positive and significantly different from 0 (D=2.20; P=0.018), consistent with the hypothesis that balancing selection (i.e., selection favoring heterozygotes) has acted on this locus. Variation in this region appears not neutral (i.e., it influences function) and diversity in the promoter region of HLA-G apparently has been maintained by natural selection.

### EXAMPLE 4

### Variation in the HLA-G Promoter Region Influences Miscarriage Rates

Patterns of Variation in the Promoter Region

Eighteen polymorphisms were present in the 1,300 bp upstream from the transcriptional start site of HLA-G in the Hutterites and 9 were identified in Chicago, causcasian, and African families (FIG. 1). Five SNPs (−201A/G, −964A/G, −140T/A, −1179G/A, and −1306A/G) were previously identified (Hviid et al. 1999); the remaining 13 SNPs are disclosed herein. Thirteen of the polymorphisms are within or very close to known transcription factor binding sites or regulatory elements. Nearly all others are either in complete LD with these sites (such as the −964A/G SNP with the −1306A/G SNP) or are in other putative binding sites. For example, the −666T allele disrupts a potential binding site for the GATA2/GATA3 transcription factors.

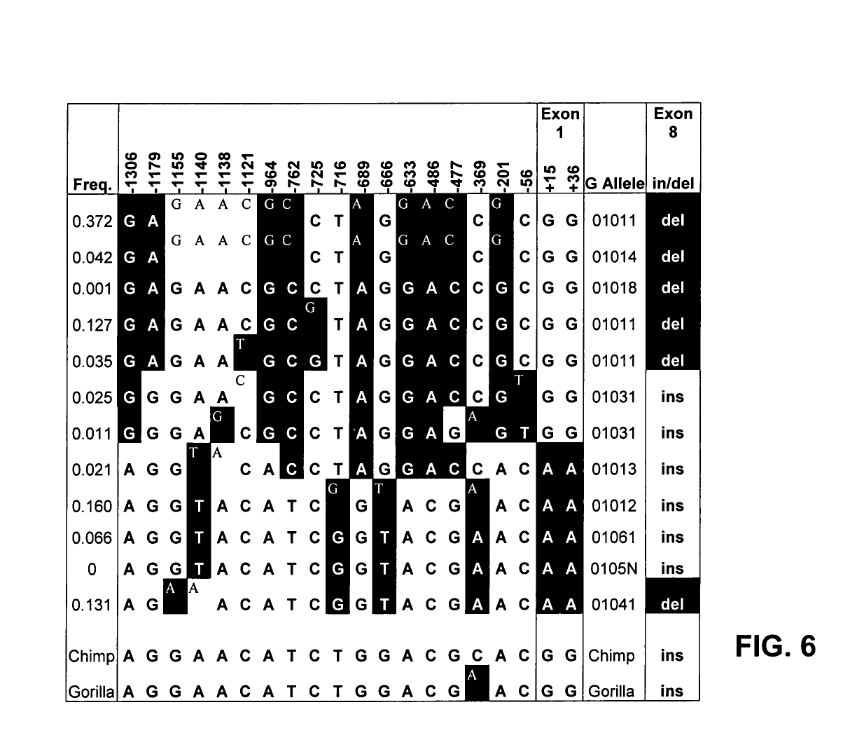

The frequencies of the alleles at each polymorphic site are shown in Table 2. Most are common polymorphisms, with minor allele frequencies >0.10. The more common allele corresponds to the chimpanzee sequence for 9 of the 27 variants and to the gorilla sequence for 8 of the variants (FIG. 6). One site (−369C/A) has an unusual pattern, in that different primate species have one or the other allele present in humans. Thus, for 8 variants, the more common allele is the ancestral allele (present in the chimpanzee and gorilla); for 10 variants, the more common allele is the derived allele (absent in the chimpanzee and gorilla); and for 1 variant (−369C/A), the ancestral state cannot be determined with certainty, because each allele is present in either the chimpanzee or the gorilla.

Overall, there is very strong LD between variants in this 1,300-bp region. Three variants that span the regulatory region, at nts −1306, −964, −201, are in complete LD in this population, with either G-G-G or A-AA occurring on each haplotype. The former corresponds to the derived and the latter to the ancestral alleles at each of these polymorphic sites. Similarly, a set of six variants clustered in the middle of the regulatory region, at nts −762, −716, −689, −666, −633, and −486, are in complete LD. These occur as either T-G-G-T-A-G or C-T-A-G-G-A. In this case, however, the haplotypes contain a mix of ancestral and derived alleles.

18 polymorphisms defined seven unique promoter haplotypes in the Hutterites (FIG. 6). In some cases, different HLA-G alleles, which are defined by variation in the coding region, share the same promoter haplotype: G*01011, G*01014, and G*01018 share one sequence, and G*01012, G*0105N, and G*01061 share another sequence. These likely represent the evolutionary relationships between alleles: the G*0105N and G*01061 alleles arose on a G*01012 haplotype background (Suarez et al. 1997; Hviid et al. 2001), and molecular studies indicated that G*0105N is a relatively young allele (Aldrich et al. 2002). On the other hand, some HLA-G alleles are associated with more than one promoter haplotype. Two variants (−1121T and −725G) define two additional G*01011 promoter haplotypes, which are associated with G*01011 alleles that are present only on HLA-A3 haplotypes. Three variants, −1138G, −477G, and −369A, define a second G*01031 promoter haplotype and are present on the only HLA-A34 haplotype in the Hutterites. On the basis of these results, exons 2 and 3 of the variant *01011 and *01031 alleles were resequenced, no additional variation was found. Six promoter variants, all with frequencies <0.20, were found exclusively with single HLA-G alleles: the −56T and −1138G alleles occur only with G*01031, the −725G and −1121T alleles occur only with G*01011, and the −1155A allele occurs only with G*01041. The remaining 13 variants were associated with more than one HLA-G allele.

Unexpectedly, the promoter haplotype associated with the G*01012, G*0105N, and G*01061 alleles is the closest to the nonhuman primate sequence, differing from the chimpanzee at only four sites (nts −1140, −716, −666, and −369) and from the gorilla at three sites (nts−1140, −716, and −666). The *01041 allele differs from the *01012 allele at only a single site in the promoter region (the derived allele −1155A). In contrast, the common *01011 allele differs from the chimpanzee at 8-10 sites and from the gorilla at 9-11 sites and is therefore the most divergent sequence, despite being the most common allele. The relatively rare *01013 allele is more similar to the ancestral sequence than are the *01011 alleles to the ancestral sequence, with only five differences from the chimpanzee and six from the gorilla. The promoter haplotypes associated with the *01031 allele are more similar overall to the *01011 alleles than to the *01012 alleles and differ from the chimpanzee at 9-11 sites and from the gorilla at 9 or 10 sites. Apart from these polymorphic sites, the human sequence differs from the chimpanzee sequence at 10 fixed sites and from the gorilla sequence at 11 fixed sites, or just under 1% of the sites surveyed, which is consistent with divergence rates between humans and chimpanzees/gorillas at other genes (Cavalcanti et al. 2002).

Analysis of Fetal Loss

On the basis of the patterns of LD among promoter SNPs and on promoter haplotype frequencies in the Hutterites, three SNPs, −689T/G, −725C/G, and −1306G/A were selected, a priori, for analyses of associations with fetal loss rates. These three SNPs, together with the 3-UTR in/del, defined five distinct groups of alleles. Because HLA-G is primarily expressed in fetal placental tissues and because tissues were not collected from aborted fetuses, HLA-G genotypes were tested in both the husbands and the wives, to determine whether transmission of a high-risk allele from either parent to the fetus would be associated with fetal loss.

The parental genotype combinations for the −1306A/−689G promoter haplotype, the −725C/G alleles, and the 14-bp in/del polymorphism are shown in Table 3. Fetal loss rates were highest among couples in which neither spouse carried a −1306A/−689G haplotype, in which both partners carried a −725G allele, and in which neither spouse carried an insertion allele (Table 3). For the logistic models, the baseline was couples in which one or both spouses carried a −1306A/−689G haplotype, neither spouse carried a −725G allele, and one or both spouses carried an insertion allele. The other groups were compared against these baselines. In the multivariable models controlling for mother's age, mother's inbreeding, and HLA-B matching, the presence of the −725G allele in both the husband and the wife was the only HLA-G polymorphism that was significantly associated with fetal loss rates (odds ratio [OR] 2.72; 95% CI 1.08-6.87; P=0.034 (Table 4). Neither the −1306A/−689G SNPs (OR 1.63; 95% CI 0.79-3.38;) nor the ins allele (OR 1.50; 95% P=186) nor the ins allele (OR 1.50; 95% CI 0.81-2.78; P=0.200) was significantly associated with risk for miscarriage, when each was considered in separate logistic models controlling for mother's age, mother's inbreeding, and HLA-B matching. The multivariate logistic regression model was fit including all three genetic variables and controlling for mother's age, mother's inbreeding, and HLA-B matching. After excluding nonsignificant (P>0.95) genetic variables, reduced to the final model with only the number of spouses carrying a −725G allele remaining (Table 4).

In the final model, the effects of matching for HLAB alleles and of mother's age on fetal loss rate were reduced and no longer significant (P>0.05). This may indicate that the presence of the −725G allele in both spouses is a more significant risk factor for fetal loss in the Hutterites than either maternal age or HLA-B matching. There was no evidence of association between maternal age and the −725G allele. However, there could be confounding between the −725G allele and HLA-B matching, because these genetic factors are associated in this sample. Also, the 95% CIs are fairly large, because of a small subgroup with the −725G allele. Further, it is not possible to determine from these data whether the risk for miscarriage is increased only in fetuses that are homozygous for the −725G allele or if heterozygous fetuses are also at risk. However, the fact that fetal loss rates are higher in couples in which only one spouse carries the −725G allele compared with those in couples with no allele (0.17 vs. 0.10, respectively) suggests that heterozygous fetuses may also be at risk, even though these differences were not significantly different herein (Table 4). Further, consistent with the modest risk associated with this allele in the Hutterites (OR 2.72; 95% CI 1.08-6.87) (Table 4), the frequencies of −725G heterozygotes and homozygotes were in Hardy-Weinberg proportions in the population.

The −1121T allele resides on a single G*01011 allele that also includes the −725G allele. Therefore, to rule out the possibility that the effect of the −725G on miscarriage is due to LD with the −1121T allele, the effects of this allele on fetal loss were examined. Because the −1121T allele is relatively infrequent in the Hutterites (frequency 0.035) and there were no couples in which both partners carried this allele, miscarriage rates were compared in couples with and without this allele. The miscarriage rates did not differ between these two groups (0.14 in couples without a −1121T allele and 0.19 in couples with a −1121T allele;) or when the P=0.56−725G allele was included in the model (P=0.97).

Further Studies of the −725G Allele

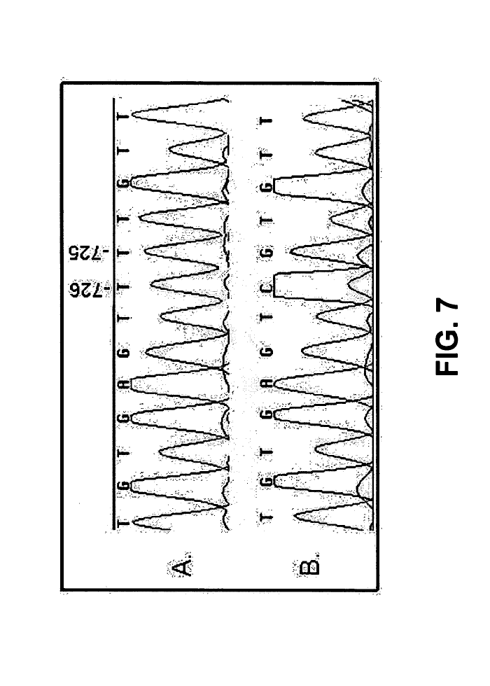

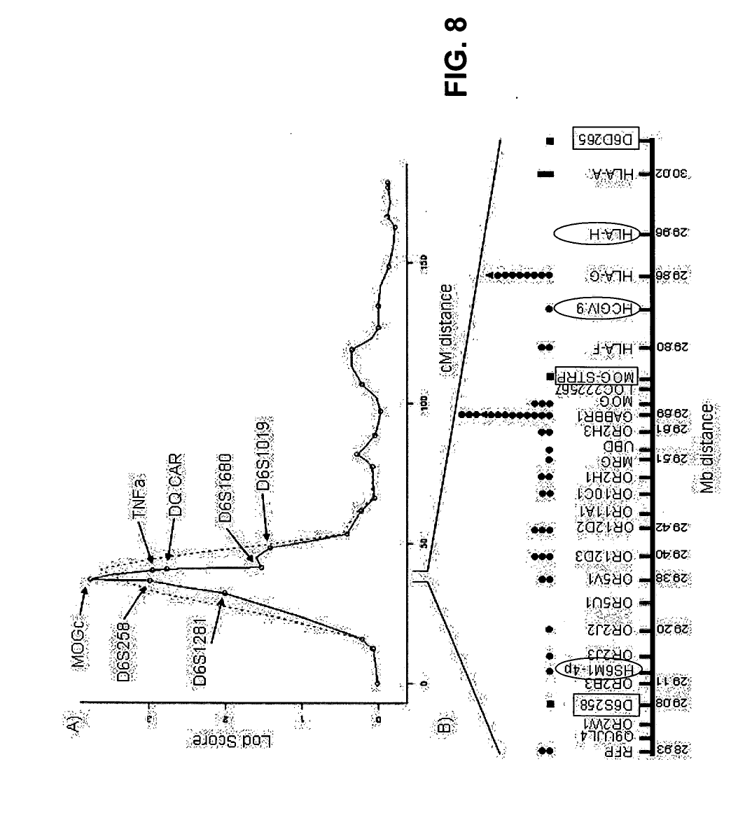

The transposition of a C to a G at position −725 creates a CpG dinucleotide at nts −726 and −725. Therefore, a question was whether −726C was methylated when associated with a G at nt −725 and perhaps influenced the binding of interferon response factor-1 (IRF-1) at the neighboring interferon-specific regulatory element binding site (FIG. 1) (Lefebvre et al. 1999). To investigate the former question, bisulfite treated DNA derived from peripheral blood cells from two individuals with the −725 CC genotype and three individuals with the −725 GG genotype (all samples are CC at nt −726) was sequenced. Bisulfite converts cytosines (C) to thymines (T) in nonmethylated DNA but does not alter cytosines that are methylated (C^{M}) (Frommer et al. 1992). After bisulfite treatment, the samples with −725 CC and −726 CC genotypes were homozygous TT at both −725 and −726, as expected in unmethylated DNA (FIG. 7A). However, the C at −726 was unconverted in samples with the −725 GG and −726 CC genotypes (FIG. 7B), although all other non-CpG cytosines in the sequence were converted to thymines (complete sequence not shown). Therefore, the −726C is methylated on −725G alleles in the majority of cells. A small T peak at this site (FIG. 8B) indicates that methylation in DNA derived from peripheral blood cells is incomplete. Nonetheless, these studies demonstrate that the allele that is associated with miscarriage in the Hutterites has a C^{M} at nt −726.

Miscarriage is the most common gestational disorder, affecting ˜15% of pregnancies. Although the majority of sporadic losses and a lesser proportion of recurrent pregnancy losses are due to chromosomal abnormalities in the fetus (Warburton and Fraser 1964; Stephenson et al. 2002), a significant proportion of both sporadic and recurrent fetal losses remain unexplained. Two studies implicated a null allele in HLA-G, *0105N, in the etiology of recurrent miscarriage, suggesting that reduced levels of HLA-G1 protein may adversely affect reproductive outcome (Aldrich et al. 2001; Pfeiffer et al. 2001). Two additional alleles, *01013 in the German study (Pfeiffer et al. 2001) and *01041 in the U.S. study (Aldrich et al. 2001), were also associated with miscarriage. A goal of the present disclosure was to determine whether additional variation that could affect levels of HLA-G protein was associated with miscarriage. The promoter region and 3′ UTR of the HLA-G gene were examined to determine where variation in these regions could directly affect transcription rates or mRNA stability, respectively, and indirectly affect levels of protein expression and pregnancy success. In contrast to the previous studies, this study was conducted in healthy women who were unselected with respect to reproductive histories but who were participants in a 15-year prospective study of pregnancy outcome. A significant association was found between a variant in the upstream regulatory region of HLA-G, −725G, which is present on a subset of G*01011 alleles and is located ˜10 bp 3′ to an IRF-1 binding motif (Lefebvre et al. 1999). Further, the transposition from a C to a G creates a CpG dinucleotide, at nts −726 and −725, that is methylated on −725G alleles.

Although the Hutterites are an isolated population, their HLA haplotypes are similar to those found in other European populations (Weitkamp and Ober 1999) and all but five rare HLA-G alleles (G*01015, G*01016, G*01042, G*01043, and G*0105N) are present in the Hutterites at frequencies similar to those in other populations (Ober et al. 1996; Ober 1997). The promoter-region haplotypes disclosed herein will also be found in other populations and associated with the same HLA-G alleles, although it is possible that additional haplotypes and even additional variation are present in outbred groups.

The two HLA-G alleles that were associated with miscarriage in previous studies, *01013 and *01041, have different promoter haplotypes, and neither carries the −725G allele. This is consistent with the fact that neither the *01013 nor the *01041 (nor the newly described *01061) allele was associated with miscarriage in the Hutterites. However, it is possible that the −725G allele is present on *01013 and *0104 haplotypes in outbred populations or that additional functional variation, which is not present in the Hutterites, is present in the promoter region of these alleles in outbred patients with recurrent miscarriage.

The extraordinary amount of variation in the 5′-upstream regulatory region of HLA-G (23 polymorphisms in 1,350 bp) was unexpected, given the relatively low levels of polymorphism in the coding region of this gene (Ober and Aldrich 1997). In particular, many of the polymorphic sites described in this study coincide with upstream regulatory elements that are unique to HLA-G. For example, two polymorphisms (−486C/A and −477G/C) are located within a heat shock element, three polymorphisms (−762T/C, −725C/G, and −716G/T) closely flank an IRF-1 binding motif, and six polymorphisms (−1306A/G, −1179G/A, −1155G/A, −1140T/A, −1138A/G, and −1121C/T) reside within the tissue-specific regulatory element, a region critical for trophoblast-specific expression of HLA-G in transgenic mice (Schmidt and Orr 1993). Thus, many of these polymorphisms, either alone or in combination, could influence transcriptional rates of HLA-G proteins in a tissue-specific manner. Although functional data showing differences in transcriptional properties of the −725G and −725C alleles is not available, the creation of a methylated CpG close to an important regulatory element on −725G alleles (Lefebvre et al. 1999) and the association between −725G and miscarriage in the Hutterites suggest that differences likely exist. A proposed functional role for the −725G allele is further supported by a recent study demonstrating that HLA-G transcription is inhibited by DNA methylation (Moreau et al. 2003). Thus, the introduction of an additional methylated cytosine on −725C alleles may downregulate transcription of HLA-G.

Alternatively, the −725G allele may be in LD with another variant that confers risk to miscarriage. It is unlikely that this variant is within the coding region of HLA-G, because the variation in the exons is identical on all *01011 alleles in the Hutterites. However, the presence of intronic variation could affect splicing, or even variation in a nearby gene that is in LD with the −725G allele. In fact, there is very significant LD between HLA-G and HLA-A alleles in all populations studied, and, in the Hutterites, the −725G allele is found exclusively on six different haplotypes that carry an HLA-A3 allele (Ober et al. 1996). Although the A3 allele per se has not been associated with miscarriage (reviewed by Ober and van der Ven 1998), Christiansen et al. (1989) have proposed that some extended HLA haplotypes carry abortion-susceptibility alleles. Thus, it is possible that the −725G allele resides on such a haplotype or even that the −725G allele is the susceptibility allele on these extended haplotypes.

The pattern of variation in the HLA-G promoter region is also remarkable. The two most common alleles, *01011 and *01012, which differ at only two silent sites in the coding region of the gene, have the most divergent promoter sequences and define two major groups of promoter haplotypes (FIG. 6). The *01012 allele is most similar to the ancestral (chimpanzee) haplotype and is identical to the promoter sequence of the *0105N and *01061 alleles, both of which arose on a *01012 background in the relatively recent past (Suarez et al. 1997; Hviid et al. 2001; Aldrich et al. 2002). The promoter region of the *01041 allele differs from the *01012 group of alleles by only a single nucleotide (−1155A). The *01011 allele, the most common allele in all populations studied, is the most divergent from the ancestral sequence and shares a common promoter sequence with the closely related *01014 and *01018 alleles. The promoter region of the more common of the two *01031 alleles differs from the more common of the three *01011 haplotype at two sites (−1179G and −56T), and two additional differences (at −1138G and −369A) define the second *01031 allele. The 5′-upstream region of the relatively rare *01013 allele is more similar to the ancestral sequence than is the *01011 allele and is more similar to the *01012 group of alleles at its 5-end (from nts −1306 to −964) than to the *01011 group of alleles. This unusual pattern may be consistent with gene conversion events reshuffling blocks of variation among haplotypes, similar to what has been observed in HLA class II genes (Zangenberg et al. 1995).

In summary, the HLA-G gene continues to reveal features that distinguish it from the other classical and nonclassical class I HLA genes. Extraordinary polymorphism exists in the upstream regulatory region of this gene and an association between miscarriage and a polymorphic variant that occurs at a frequency of 0.16 in the study population. This variant flanks a binding site for the transcription factor IRF-1 and creates a methylated CpG dinucleotide, which could alter the conformation of DNA and affect IRF-1 binding. It is not yet known if this variant allele influences the transcriptional properties of HLA-G or which of the HLAG isoforms might be affected. Nonetheless, the association with miscarriage suggests that the −725G allele may downregulate transcription of HLA-G and, like the *0105N null allele, result in reduced protein expression. Thus, the likelihood of a successful pregnancy may be determined, in part, by either the absolute levels of HLA-G protein or the relative levels of the different protein isoforms, further suggesting a critical role for this unusual gene throughout pregnancy.

### EXAMPLE 5

### Fine Mapping and Positional Candidate Studies Identify an Asthma Susceptibility Locus in the HLA Region on Chromosome 6p21

A genome-wide screen was conducted in families who participated in the Collaborative Study on the Genetics of Asthma (CSGA). The strongest linkage signal in 129 Caucasian families was on chromosome 6p21 at marker D6S1281 (Lod=1.91, P=0.003), 2.5 cM telomeric to the human leukocyte antigen (HLA) complex. All the evidence for 6p linkage was in the 35 Caucasian families ascertained in Chicago (Lod=3.6) (FIG. 8A). Subsequent studies were conducted in these families, and in 46 Caucasian asthmatic child-parent trios also ascertained in Chicago and two other populations that had previously shown evidence of linkage with asthma-associated phenotypes to markers in this region.

To further narrow the linked region, the Chicago families were genotyped for five additional short tandem repeat polymorphisms (STRPs), two that reside within the HLA region (DQ.CAR and TNFa) and three flanking markers (D6S258, MOGc, and D6S1680) (FIG. 8A). The lod score in these families increased to 3.8, peaking at MOGc. The information content of these markers for linkage was 95%, indicating that the lod score could not increase nor improve the resolution by adding more markers. Further, the MOGc 136 bp allele was overtransmitted to asthmatic children in the families (42 transmission [TR]: 23 nontransmission [NT]; P (corrected for relatedness and number of alleles)=0.06). In the trios, the MOGc 134 bp allele was overtransmitted to asthmatic children (17 TR: 6 NT; P<0.05), suggesting that the susceptibility gene (or genes) resides on different haplotype backgrounds in the families and in the trios. Because of the extensive LD in the HLA region, the disease locus could have been located at a far distance despite the evidence for association between MOGc alleles and asthma. To determine the extent of the shared haplotype among affected individuals, the families were genotyped at the HLA-A locus and at six STRP loci spanning the HLA class I region between TNFα and HLA-A. Both the HLA-A locus and the remainder of the HLA region proximal to were excluded.

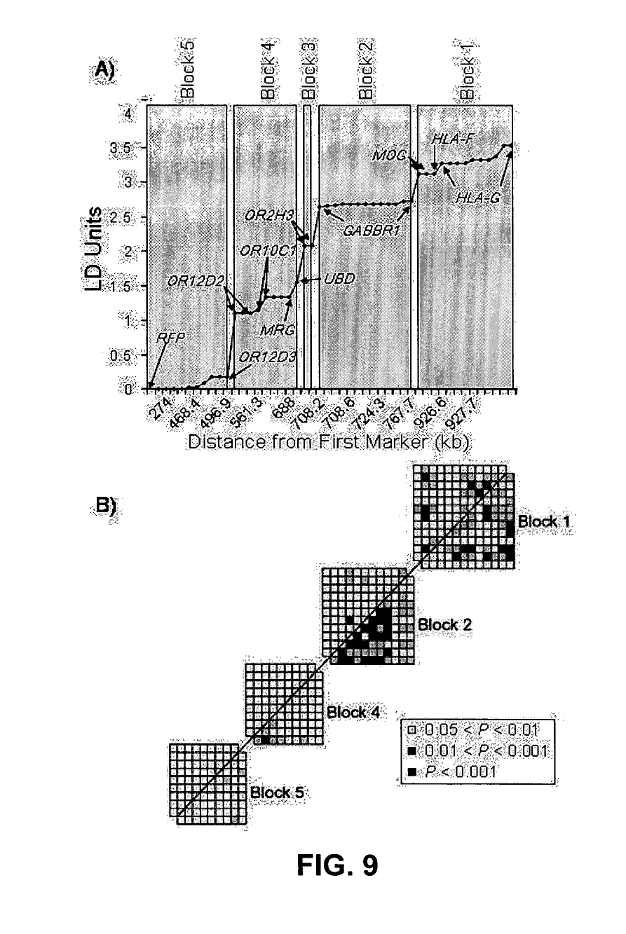

Chromosome 6p21 is one of the best characterized regions in the human genome and the nucleotide sequence between D6S258 and D6S265 is known. This region is gene rich with 19 known or predicted genes and at least 30 pseudogenes in the 1 Mb region from HLA-A to OR2B3 (FIG. 9B). To localize the susceptibility-associated variation in this region, the families and trios for an additional 48 polymorphisms in 15 genes and two pseudogenes (FIG. 8B; were genotyped).

To determine whether any of these variants explained some or all of the original evidence for linkage in the families by conditioning on the genotype at each polymorphic site was performed and the evidence re-examining for linkage. Multipoint analysis conditional on the genotypes of one variant in the HLA-G gene (1489C/T, His93His) yielded a lod score of 0.9, which is substantially lower than the maximum lod score in this region. Analyses conditional on the other variants led to lod scores larger than 0.9. Thus, the 1489C/T polymorphism accounted for most of the linkage in the families; the remaining evidence for linkage could be due to chance sharing among affected family members or to the presence of a second susceptibility locus in the linked region. Nonetheless, it was unexpected that conditioning on a single SNP could result in a reduction in the lod score of this magnitude.

The pattern of LD was examined across this region in unrelated individuals from the families and trios and identified five LD blocks (FIG. 9A). To determine which LD block contains variation that contributes to asthma susceptibility, pairwise combinations of SNPs within each block by the transmission disequilibrium test (TDT), conditional on the evidence for linkage at each position. Only pairwise combinations of SNPs in block 1 showed significant nonrandom transmission of haplotypes (P<0.001) in both the families and trios (FIG. 9B).

Analyses of the individual variants revealed that four polymorphisms in HLA-G (block 1) were associated with asthma in both the families and trios; SNPs in two genes in block 4 (OR12D2 and OR10C1) and one gene each in block 2 (GABBR1) and block 5 (OR5V) were associated with asthma in the families only. Thus, the HLA-G locus in block 1 was the only locus in the region that showed evidence for association with asthma in both the families and trios. However, in the families the association was with a haplotype carrying the −964G allele (36 TR: 20 NT), whereas the association in the trios was with a different haplotype carrying the −964A allele (23 TR: 11 NT).

To further localize and characterize the susceptibility locus (or loci), selected markers were genotyped in two populations that previously showed linkage of asthma-related phenotypes to 6p21: the Hutterites, a founder population of European descent, and Dutch families. Because of the different ascertainment schemes, different statistical approaches were used in each sample. Ascertainment in the Hutterites was population-based; individuals in this single, large pedigree were not selected on the basis of any particular phenotype. As a result, a test of association was used that was designed for large, multigenerational pedigrees. (Bourgain et al., 2003). Here, cases were defined as individuals with BHR (N=156), and controls as individuals with a negative history of asthma symptoms and without BHR (N=434). Four SNPs were identified in the HLA-G gene that showed evidence for association with BHR (P<0.05). Only one other variant (of 21 genotyped) showed any evidence for association with BHR (OR12D2*1; P=0.05). In the Hutterites, the −964G allele was associated with BHR, similar to the results in the Chicago families.

The 200 Dutch families were ascertained through a parent with asthma. Therefore, these data were analyzed stratified by mothers' and fathers' affection status (BHR+ or BHR−). The −964A allele was overtransmitted to children with BHR if the mother was unaffected (57 TR: 27 NT; P=0.004), whereas the −964G allele was overtransmitted to children with BHR if the mother was affected (61 TR: 45 NT; P=0.15). The differences in transmission patterns of alleles to BHR children of mothers with and without BHR was highly significant (P=0.0008). Similar analyses stratifying by father's asthma status did not show a significant trend. Table 5 shows the HLA-G −964 genotype-specific prevalences for BHR and atopy in Dutch children by mother's affection status. The prevalence of atopy is not influenced by maternal BHR status, although it differs by genotype: 60% of AA children, 49% of AG children, and 41% of GG children are atopic (P=0.006). In contrast, the prevalences of BHR are not different if maternal status is ignored, but the prevalence of BHR among children with the GG genotype is significantly influenced by maternal status. Among children of BHR mothers, 56% of GG children are BHR; among children of non-BHR mothers, 26% of GG children are BHR (P=0.001). Thus GG children are less likely to be atopic, but more likely to be BHR if their mother is also BHR. A similar trend was observed in the Hutterites: the frequency of the −964G allele was higher (0.69) among BHR children of BHR mothers than among BHR children of non-BHR mothers (0.60), although this difference was not significant (P=0.16).

Thus, in the Chicago families and trios, and in the Hutterite and Dutch families, variants in the HLA-G gene were associated with asthma or BHR. Although no other variation examined in this region showed associations in all four populations, it is possible that unidentified variation in HLA-G or in other genes in block 1 also contributes to susceptibility. Further, because different alleles and haplotypes were associated in the different populations and also when stratified by maternal affection status, susceptibility at this locus is complex, is influenced by maternal factors, and may be associated with multiple related phenotypes.

The association of HLA-G variants with asthma is particularly intriguing because asthma, like pregnancy, is characterized by a predominance of Th2 cytokines. Further, the interaction between maternal factors and child's HLA-G genotype in the Dutch and Hutterite families is notable given the important role that this gene plays in pregnancy and that maternal asthma is a well established risk factor for the development of asthma.

To evaluate whether the HLA-G gene could contribute toward the immunologic milieu in the asthmatic lung, the expression pattern of HLA-G was examined in lung tissues from one asthmatic and one non-asthmatic individual. The strong expression of HLA-G in bronchial epithelial cells in the asthmatic lung, was demonstrated by immunohistochemistry whereas expression was less in the non-asthmatic lung (FIG. 5). Expression in the lung was limited to the soluble isoform, HLA-G5 (also called soluble G1). Neither the transmembrane G1 and G2 isoforms nor the soluble G6 isoform were identified in these tissues. Thus, expression of HLA-G in the lung might contribute towards the aberrant immunologic response to inhaled allergens in genetically susceptible individuals. Because SNPs in the HLA-G promoter region were associated with asthma or BHR in all populations studied, these polymorphisms may influence transcription of HLA-G.

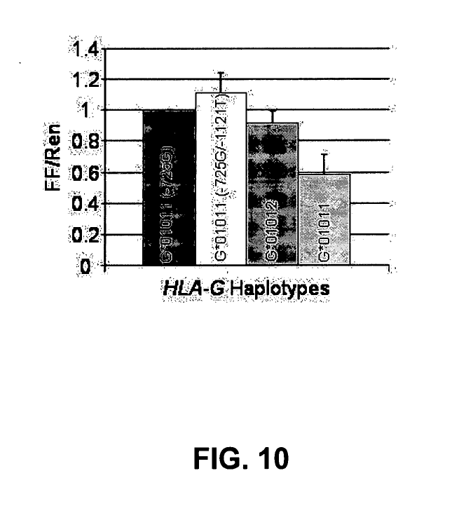

Four promoter region haplotypes were used in a luciferase assay (FIG. 10). There were significant differences in basal expression levels between these four haplotypes (ANOVA, P=0.005), indicating that variation in the promoter region of HLA-G influences luciferase transcription in a human bronchial epithelial (HBE) cell line. Unexpectedly, the haplotype that was associated with asthma or BHR in the Chicago families, in Hutterites, and in Dutch children of mothers with BHR showed the lowest expression levels in this assay. Although the relative expression levels of these haplotypes may differ considerably under biological conditions and/or during an inflammatory response, these studies indicate that the variation associated with asthma or BHR in four populations also influences the transcriptional properties of this gene.

Although the LD pattern in this region does not rule out variation in other genes in block 1 as contributing to susceptibility, several lines of evidence suggest that HLA-G is at least one of the asthma susceptibility loci in this region. First, a single SNP in HLA-G (1489C/T) accounts for nearly all the linkage in this region. For other variation to be causal, it would have to be in nearly perfect LD with and at similar frequency to the HLA-G 1489C/T SNP and not in LD with the other SNPs tested. Further, if the effect size of the initial linkage signal was biased upwards due to chance sharing in the families, then HLA-G may be the sole asthma susceptibility locus in this region. Even if the signal was not biased, additional undetected variation in HLA-G could also contribute to risk. Second, among the variants that were surveyed across the linked region, only SNPs in HLA-G were associated with asthma or BHR in four different populations, which were ascertained using different strategies. Third, the HLA-G soluble isoform, G5, was highly expressed in bronchial epithelial cells in an asthmatic lung. The presence of soluble HLA-G protein in bronchial epithelial cells indicates that it could participate in a local inflammatory response to airborne allergens or other antagonists. Fourth, in vitro expression assays revealed significant differences between haplotypes carrying different alleles in the HLA-G promoter region, suggesting the potential for genotype-specific responses to antagonists. Based on these combined data, HLA-G is likely an asthma susceptibility locus on chromosome 6p21.

In addition to asthma and associated phenotypes, many other inflammatory diseases, such as multiple sclerosis, psoriasis, atopic dermatitis, inflammatory bowel disease, and schizophrenia, have been linked to the HLA region. To date, the variation underlying these linkages has not been identified. Variation in the HLA-G gene may contribute to susceptibility to a wide range of inflammatory diseases, including asthma. Thus, this gene that likely evolved to promote tolerance in pregnancy, may contribute to risk for many common diseases, suggesting that novel therapeutic strategies could have broad relevance to these immune-mediated diseases.

### Materials and Methods

1. HLA-G and Reproduction

Population

The Hutterites are an Anabaptist sect that originated in the Tyrolean Alps in the 1500s and settled in what is Ober et al.: HLA-G and Miscarriage 1427 now South Dakota in the 1800s (Steinberg et al. 1967; Hostetler 1974). The >35,000 extant Hutterites are descendants of >90 founders who lived in the early 1700s to early 1800s (Martin 1970). The Hutterites of South Dakota have participated in our studies of HLA and fertility since 1982. These individuals live on 36 communal farms (called colonies) and are descendants of only 64 of the 90 Hutterite founders (Ober et al. 1997). As a result of the small number of founding genomes, only 67 unique HLA haplotypes are present in the population (51 ancestral and 16 recent recombinant haplotypes) (Weitkamp and Ober 1999). In addition, the Hutterites' traditional proscription of contraception and a naturally high fertility rate resulted in large families (median completed sibship size was 10 in 1965) and relatively few (2%) childless couples (Sheps 1965; Ober et al. 1999).

In 1986, a prospective study was initiated to assess the relationship between parental HLA types and pregnancy outcome in the South Dakota Hutterites (Ober et al. 1992, 1998b). The women in this study are provided with calendar diaries and EPT pregnancy test kits (kindly provided by Warner-Lambert). They record in the diary dates of menses, changes in nursing patterns, illnesses or travel for the husband and wife, and dates of miscarriages or deliveries. In addition, they are instructed to test for pregnancy if they do not start menses exactly 1 mo. after the 1 st d. of their previous period and to record the results of all pregnancy tests in the diaries. They are also asked to start testing for pregnancy on a monthly basis starting 6 mos. after delivery, until menses resumes. Results of all pregnancy tests and outcomes of each pregnancy are recorded in the diaries, which are collected yearly, either in person or through the mail. The results reported here include data collected through 2001, representing 15 years of study.

The sample included 474 pregnancies that were either miscarried or followed beyond 20 wk. of gestation in 191 women. The fetal loss rate in this sample was 15.6% (74 losses in 474 pregnancies), which is nearly identical to estimates of clinically recognized miscarriage rates in outbred populations (Wilcox et al. 1988). All women with fetal losses also had successful pregnancies, and none would be considered to have recurrent miscarriage (Aldrich et al. 2001; Pfeiffer et al. 2001). Of these 191 women, genetic data were available for both partners in 162 couples, providing 403 pregnancies.

Haplotype Assignments and Polymorphism Detection in the Hutterites

HLA haplotypes were initially characterized in 1,045 Hutterites by the direct observation of alleles segregating in families at five serologically-typed loci: HLA-A, HLAB, HLA-C, HLA-DR, and HLA-DQ, (Kostyu et al. 1989). HLA haplotypes were inferred in an additional 216 individuals on the basis of the haplotypes present in their children, spouse, siblings, and/or parents. The subsequent molecular and biochemical genotyping of 21 HLA region loci, including HLA-G (Ober et al. 1996), has been conducted in a sample of 85 Hutterites selected to represent all of the 51 ancestral haplotypes present in the Hutterites of South Dakota (Weitkamp and Ober 1999). In these ˜80 individuals, all haplotypes were represented by at least three Hutterites who were selected from distant branches of the pedigree, with the exception of eight rare haplotypes that were represented by two (N=7) or one (N=1) individual. Using this approach, the alleles on each haplotype were determined at 21 loci and then assigned these alleles to all Hutterites with that particular haplotype, as described by Weitkamp and Ober (1999). More recently, HLA haplotypes were assigned to an additional 730 Hutterites by molecular genotyping at informative loci and inferring haplotypes from family data. Among the 324 spouses (162 couples) in this study, haplotypes were determined by serology in 217 and by molecular genotyping and inference in 107.

For studies of polymorphisms in the HLA-G promoter region, a sample of 42 Hutterites was selected who carried 47 of the 51 ancestral HLA haplotypes and all eight HLA-G alleles present in the population. These 42 individuals were not selected on the basis of reproductive history. Alleles could not be assigned to four ancestral haplotypes, because there were insufficient amounts of DNA from individuals representing these rare haplotypes. In addition, studies included DNA from a women who was homozygous for the null G*0105N allele (Ober et al. 1998a), because this allele is not present in the Hutterites (Ober et al. 1996). Studies of the exon 814-bp in/del alleles in the Hutterites were described elsewhere (Ober et al. 1996). For surveying the 5′-upstream promoter region, DNA from all individuals was PCR-amplified using primers that generated a 1,752-bp fragment from −1477 bp (5′) to +208 (3′) relative to the transcriptional start site: 5_-ACATTCTAGAAGCTTCACAAGAATG and 3′-TGGGCCTTGGTGTTCCGTG. The PCR product was sequenced in both directions by Big Dye Terminator v.3 (Applied Biosystems) on an ABI 3100 Automated Sequencer (Applied Biosystems), using the PCR primers as sequencing primers as well as six internal primers:

G-908-TTCACCTCACAGTTGTAAGTGTTC,
G-830F-CACACGGAAACTTAGGGCTACG,
G-1123F-GCCTCGCTGGGTGTTCTTTGC,
G-304RGCCAAGCGTTCTGTCTCAGTGT,
GPR-247-TCAAGCGTGGCTCTCAGGGTC,
and
GIN1-98-GTTTCCCTCCTGACCCCGCACT.

In addition, the sequence of the promoter haplotype was determined in cloned DNA from 17 heterozygous individuals. For these experiments, PCR-amplified DNA was cloned into pCR 4-TOPO vector Invitrogen) and was sequenced using the same protocols described herein. DNA from two chimpanzees (Pan troglodytes) and two gorillas (Gorilla gorilla) was also sequenced, to determine the ancestral allele at each polymorphic site. The human BAC clone AP000521.1 was used as the reference sequence.

Alleles at each of the polymorphic sites were assigned to 47 Hutterite haplotypes, as described above. Allele frequencies were determined from the haplotype frequencies, which were estimated from a sample of 962 married Hutterites representing all four lines of colony descent (Ober et al. 1997), as described elsewhere (Ober et al. 1996; Weitkamp and Ober 1999).

Methylation Studies

To determine whether the −725C/G SNP has different methylation patterns associated with each allele, bisulfite-treated DNA was sequenced from individuals with different genotypes at this SNP. Genomic DNA was treated with bisulfite, following published protocols (Olek et al. 1996; Warnecke et al. 2002) with minor modifications. In brief, 100 ng of DNA was pretreated with proteinase K (1 mg/ml; 37° C. for 18 h) prior to treatment with 1 ml of bisulfite solution (5.7 g of sodium metabisulphite, 165 mg of hydroquinone, 2.25 ml of 2 M NaOH, and 9 ml of ddH20). Tubes with 500 ml of mineral oil were kept at −20° C. for 10-20 min. prior to adding agarose beads (Olek et al. 1996); after adding the beads, they were kept on ice for an additional 10-20 min. PCR primers were designed following the suggestions of Warnecke et al. (2002) and were as follows: forward,AGGAGATGTTTTGGATTTAT; and reverse, AAATAACATAACCTTAATAACC. These same primers were used for sequencing, as described herein.

Statistical Analysis

The relationship between polymorphisms in the HLAG gene and fetal loss was assessed in Hutterite couples participating in the prospective study, through use of a generalized estimating equation logistic regression model (chapter 8, section 8.2 of Diggle et al. 1994). This model adjusts for the clustering of pregnancies within couples (Hauck and Ober 1991). The models were fit using STATA software (STATA Corporation).

To minimize the number of comparisons, promoter-region SNPs were selected for the analysis of fetal loss that both captured the patterns of LD and identified the major haplotype groups. To do this, the smallest number of SNPs were identified that, together with the exon 8 in/del, differentiated the four haplotypes with frequencies >0.10. One SNP from each of the two haplotype blocks disclosed herein:—1306G/A and −689A/G. These two SNPs, along, with the in/del, identified all haplotypes with frequencies >0.10 except one, which was differentiated from the others by the −725C/G/T SNP. Taken together, these four variants defined six haplotype groups and were the smallest number of variants required to identify all haplotypes with frequency >0.10. Given the large number of possible combinations of haplotypes in the husbands and wives in the sample, couples were classified as to the number of spouses per couple (on a scale of 0-2) that carried a −1306A/−689G (AG) haplotype, the −725G allele, or an insertion (ins) allele. This selection and categorization process was performed prior to examining the effects of these polymorphisms on fetal loss rates. In the multivariable logistic model, the effects of the −1306A/−689G (AG) haplotypes, the −725G allele, and 14-bp ins allele were examined. In all analyses, variables were controlled that are known to affect fetal loss in the Hutterites. These include maternal age and HLA-B matching (Ober et al. 1998b); maternal inbreeding was also included because it provided more precise estimates for the covariates. Neither inbreeding in the father nor relatedness between the parents influences fertility in this population (Ober et al. 1998b, 1999). The final model was reduced to include only significant genetic variables.

Electronic-Database Information

The URL for data presented herein is as follows:
Online Mendelian Inheritance in Man (OMIM), http:/www.ncbi.nlm.nih.gov/Omim/(for HLA-G)
   2. HLA-G and Asthma
   Sample Ascertainment

Details of the protocols used to evaluate the CSGA families are in Lester et al. (2001), Ober et al. (2000), Hutterites, and Dutch families (Pankuysen et al., 1995). Briefly, CSGA families were ascertained through two siblings with asthma and were extended to include other affected relatives, but never skipping through more than one unaffected relative. The 35 Chicago families ranged in size from 4 to 18 (mean family size 7). Asthma was diagnosed as follows: 1) either a) fall in baseline FEV1 by 20% at 25 mg/ml methacholine or b) 15% increase in baseline FEV1 after bronchodilator use; c) 2 of the following symptoms: cough, wheeze or dyspnea; 3) current medication use or doctor's diagnosis of asthma. All participating relatives of the siblings were studied. Trios were individuals meeting the same criteria as the CSGA asthmatics and their parents (N=46). 693 Hutterites who are related to each other in a 13-generation, 1,623-member pedigree were evaluated using a modified CGSA protocol. BHR was defined as a fall in baseline FEV1 by 20% at <25 mg/ml methacholine. The 200 Dutch families included 598 individuals with BHR (out of a total of 1,183 individuals). BHR was defined as fall in baseline FEV1 by 20% at 32 mg/ml histamine. Atopy was defined as a positive skin prick test to airborne allergens. These studies were approved by the Institutional Review Boards at each institution.

SNP Selection

One common SNP was genotyped every 10-20 kb across each gene the region and all nonsynonomous SNPs, when possible. SNPs were selected from dbSNP (http://www.ncbi.nlm.nih.gov/SNP/) or discovered. In this gene, SNPs that identify clusters of variants that are in perfect or near perfect LD or uniquely define all of the common HLA-G alleles (>5% in the Hutterites).

Immunohistochemistry

Human donor lungs that could not be used for transplantation were obtained under an IRB-approved protocol from the Regional Organ Bank of Illinois. Bronchi were dissected and frozen in OCT. 5 micron sections were stained for antibodies against HLA-G1, -G2, -G5, and -G6 isoforms, as described. by Morales et al., (2003) Slides were read without knowledge of the affection status of the donor.

Expression Assay

A 1379 bp fragment (−1412 to −33 relative to the translational start site) from the known HLA-G promoter was PCR-amplified from individuals carrying each of four unique promoter haplotypes (Ober et al., 2003) using the following primers: forward CAC-GGTACC-ACTGGAGTGTTTTAGGTGGAGA, reverse CAC-CTCGAG-GTGAGCGAGGACTTTAGAACCA. The PCR products were cloned into the promoterless pGL3-basic vector, upstream of the firefly luciferase reporter gene (Promega, Madison, Wis.). 1 μg of the reporter construct (or pGL3 empty vector) was transfected into subconfluent 16HBE cells. Transfection efficiency was normalized by co-transfecting 12 ng of pRL.SV40 Renilla luciferase vector (Promega). Cells were harvested and lysed 40 hours post-transfection. The firefly and renilla activities were assayed using a luminometer and Promega's Dual-Luciferase Reporter Assay System. HLA-G promoter activity was determined by dividing firefly relative light units (RLUs) by renilla RLUs. Transfections were performed in triplicate, and normalized luciferase activity was averaged among wells and then standardized to the G*01011 (−725G) haplotype to provide the data for each experiment.

Statistical Analyses

Linkage analyses were performed with the program ALLEGRO (Gudbjartsson et al., 2000) using affecteds-only allele-sharing methods. The exponential allele-sharing model was used on the scoring function Spairs (Kong and Cox, 1997). The identity-by descent (IBD) status was determined using multipoint calculations. P-values are calculated based on large-sample approximations. The TDT was performed on all the affecteds in the families for whom both parents were genotyped. (Spielman et al., 1993). The chi-square statistic was used, and in markers with more than two alleles the maximum of the individual chi-square functions was used as the test statistic. In the datasets where there were more than two affected individuals per family, the significance of the test was evaluated using simulations. The simulations were performed conditional on the IBD process at that location. The missing genotype patterns were identical in the simulated and observed datasets.

Two-marker TDT was performed for selected pairs of markers within each of five blocks, defined so that markers in different blocks have small estimated LD measures. This procedure leads to a smaller number of tests, and therefore to a less stringent threshold of significance. Only trios without missing genotypes for the pair of markers were used. The transmission counts were estimated based on the 2-marker haplotype frequencies that were calculated from unrelated individuals. The test statistic for one pair of markers is the maximum over all the haplotypes of (t-n)²/(1+n+t), where t is the estimated number of transmissions, and n is the estimated number of non-transmissions of the haplotype from heterozygous parents. The statistical significance was evaluated by simulation. Founder haplotypes were simulated within each block of markers, and Mendelian transmission assuming no recombination were simulated conditional on the IBD process. The missing genotype patterns were identical in the simulated and observed datasets. Two-marker analyses, identical to those disclosed herein, were performed in each simulated dataset. The P-values are calculated as the number of simulated datasets where a larger test statistic was found. The difference in prevalences was calculated using chi-square statistics. P-values were obtained by simulating conditional on the pedigree structure and the IBD process.

TABLE 1
Summary of results of luciferase experiments in JEG-3 cells. Statistics are provided
only for treatments with at least 4 independent experiments (each performed in triplicate).
Significant differences between haplotypes or after treatment are bolded.

| | No. | |
|---|---|---|
| Treatment | Experiments | Results |
| None | 8 | Significant differences between promoters (P < 0.001) after 40 hours of culture, |
| | | with higher levels associated with promoters with −725G allele at 24 and 40 hours. |
| Heat Shock | 2 | Large increase in expression; possible larger relative response of the common G*01011 |
| | | promoter and smaller relative response of the G*01012 promoter. |
| Low Oxygen (2%) | 2 | Large increase in expression; no obvious differences in promoter-specific response. |
| Chemical hypoxia (CoCL2) | 4 | Large increase in expression; no obvious differences in promoters-specific responses. |
| Demethylation with | 2 | Increase (<2-fold) of all promoters; no obvious differences in promoter-specific responses. |
| 5′-azacytidine | | |
| IL-10 | 2 | Small effects on expression of promoters with −725G allele and a decrease in expression |
| | | of the common *01011 and *01012 promoters. |
| TRAIL | 4 | Decrease in expression of all promoters in a dose-dependent fashion; common G*01011 |
| | | promoter showing significant decrease at 500 ng (P = 0.009). |
| LIF | 1 | Small increases in expression (<2-fold) of all promoters. |
| IFN-β | 1 | Large (˜3-fold) increase in expression of all promoters. |
| TGF-β | 1 | Possible promoter-specific (promoters with −725G show little change from |
| | | aseline whereas the common G*01011 and G*01012 promoters show increase). |
| TNF-α | 1 | Small decreases in expression of all promoters. |
| IL-1β | 1 | No obvious effects on expression of any promoters. |
| IL-13 | 1 | No obvious effects on expression of any promoters. |
| IL-4 | 1 | No obvious effects on expression of any promoters. |

TABLE 2
HLA-G Promoter SNPs. Frequency of the minor allele (underlined) at HLA-G
promoter SNPs and one exon 1 SNP (+15G/A) in the Hutterites (HT) and in outbred
EA (N = 47) and AA (N = 44) control subjects. The −922C/A SNP was present only in 3
asthma patients. Nt positions are relative to the translational start site.

| nt | | | | | | | | | | | | | |
|---|---|---|---|---|---|---|---|---|---|---|---|---|---|
| position | −1306 | −1179 | −1155 | −114C | −1138 | −1121 | −964 | −922 | −810 | −762 | −725 | −716 | −689 |
| SNP | G/A | A/G | G/A | A/T | A/G | C/T | G/A | C/A | C/T | C/T | C/G/T | T/G | G/A |
| Freq. in | 0.36 | 0.41 | 0.13 | 0.25 | 0.011 | 0.035 | 0.38 | 0 | 0 | 0.36 | 0.16/0.036 | 0.36 | 0.36 |
| HT | | | | | | | | | | | | | |
| Freq. in | 0.50 | 0.48 | 0.053 | 0.45 | 0.022 | 0.053 | 0.50 | 0 | 0.011 | 0.50 | 0.12/0.022 | 0.50 | 0.50 |
| EA | | | | | | | | | | | | | |
| Freq. in | 0.55 | 0.35 | 0.24 | 0.31 | 0.10 | 0 | 0.55 | 0 | 0 | 0.55 | 0.07/0.10 | 0.55 | 0.45 |
| AA | | | | | | | | | | | | | |
| nt | | | | | | | | | | | | | |
| position | −666 | −646 | −633 | −509 | −486 | −483 | −477 | −400 | −391 | −369 | −201 | −56 | +15 |
| SNP | G/T | A/G | G/A | C/G | A/C | A/G | C/G | G/A | G/A | C/A | G/A | C/T | G/A |
| Freq. in | 0.36 | 0.011 | 0.36 | 0 | 0.36 | 0 | 0.35 | 0.036 | 0.036 | 0.35 | 0.38 | 0.036 | 0.036 |
| HT | | | | | | | | | | | | | |
| Freq. in | 0.50 | 0.011 | 0.50 | 0.022 | 0.50 | 0.021 | 0.52 | 0.022 | 0.022 | 0.52 | 0.50 | 0.022 | 0.50 |
| EA | | | | | | | | | | | | | |
| Freq. in | 0.55 | 0.045 | 0.55 | 0.068 | 0.55 | 0.057 | 0.65 | 0.068 | 0.068 | 0.65 | 0.55 | 0.10 | 0.55 |
| AA | | | | | | | | | | | | | |

TABLE 3
Parental Genotype Combinations for the 51306G/A and 5689A/G
Haplotypes, the 5725C/G Polymorphism, and the 14-bp in/del
Polymorphism, in Hutterite Couples

| | | | |
|---|---|---|---|
| No. of Spouses | | | |
| Loss | No. of | | Fetal |
| per Couple | Pregnancies | Genotype Combinations in Couples | Rate |

With
−1306A/−689G
haplotype:

| | | | |
|---|---|---|---|
| 0 | 68 | nonAG/nonAGx × nonAG/nonAG | .22 |
| 1 | 182 | AG/nonAG × nonAG/nonAG | .13 |
| | | or AG/AG × nonAG/nonAG | |
| 2 | 153 | AG/nonAG × AG/nonAG, | .13 |
| | | AG/AG × AG/nonAG or | |
| | | AG/AG × AG/AG | |
| With | | | |
| −725G allele: | | | |
| 0 | 186 | CC × CC | .10 |
| 1 | 188 | CC × CG or CC × GG | .17 |
| 2 | 29 | CG × CG or CG × GG | .28 |
| With exon 14 | | | |
| ins allele: | | | |
| 0 | 101 | del/del × del/del | .21 |
| 1 | 190 | ins/del × del/del or | .11 |
| | | ins/ins × del/del | |
| 2 | 112 | ins/del × ins/del, ins/ins × ins/del, | .16 |
| | | or ins/ins × ins/ins | |

TABLE 4
Analysis of Fetal Loss in the Hutterites

| Variable | P | OR | 95% CI |
|---|---|---|---|
| Mother's age | .061 | 1.07 | .99-1.15 |
| Mother's inbreeding | .751 | .96 | .80-1.18 |
| HLA-B matching | .056 | 1.85 | .98-3.34 |
| −725G in one spouse | .1161 | .71 | .88-3.34 |
| −725G in both spouses | .034 | 2.72 | 1.08-6.87 |

TABLE 5
HLA-G genotype, risk for asthma, and maternal affection status.
A) Number of Dutch children with BHR and atopy by child's genotype
and mother's affection status. The prevalence of atopy is influenced by
the child's genotype, but not the mother's affection status
(P = 0.006 for differences between genotypes). The prevalence of
BHR is influenced by both the child's genotype and the mother's
affection status. Among GG children the prevalence of BHR is 56% if
the mother is BHR and 26% if the mother not BHR (P = 0.001). B)
Number of children with asthma in the Chicago families by child's
genotype and mother's affection status. The genotype distributions in
the children differ by mother's affection status. The −964GG genotype
is more common among asthmatic children of BHR mothers, whereas the
−964AA genotype is more common among asthmatic children of
non-BHR mothers, although these differences do not reach statistic
significance. Genotype differences are more striking for HLA-G* 1489
(difference between asthmatic children of BHR+ and BHR− mothers;
P = 0.009), the same SNP that explained the evidence for linkage.
A)

| Child's HLA- | | | | | |
|---|---|---|---|---|---|
| G −964 | Mother's | Child's Affection Status | | | |
| Genotype | Status | BHR+ | BHR− | Atopic | Non-Atopic |
| AA | BHR+ | 22 | 28 | 33 | 18 |
| AA | BHR− | 27 | 30 | 32 | 25 |
| AG | BHR+ | 57 | 63 | 62 | 59 |
| AG | BHR− | 56 | 68 | 60 | 67 |
| GG | BHR+ | 57 | 45 | 44 | 61 |
| GG | BHR− | 18 | 50 | 28 | 44 |

B)

| | HLA-G −964 | | | HLA-G 1489 | | |
|---|---|---|---|---|---|---|
| Mother's | Genotype | | | Genotype | | |
| Status | GG | AG | AA | CC | CT | TT |
| BHR+ | 12 | 17 | 4 | 18 | 15 | 0 |
| BHR− | 9 | 19 | 18 | 9 | 30 | 12 |

TABLE 6
TDT in asthma trios in CSGA families and trios and in Dutch
families; case-control test in Hutterites. Associations are with asthma in the
Chicago families and trios and with BHR in the Hutterite and Dutch families. P-
values <0.05 are shown in bold.

| | | | | P-value | | | | |
|---|---|---|---|---|---|---|---|---|
| | | | | | | | Dutch Families | |
| | Mb from | | dbSNP | Chicago | Chicago | | Mother | Mother |
| Locus | p-ter | Polymorphism | rs# | Families | Trios | Hutterites | BHR− | BHR+ |
| RFP*2 | 28.98 | C/T | 209131 | 0.778 | 0.715 | — | — | — |
| RFP*1 | 29.03 | A/G | 1237485 | 0.641 | 0.862 | — | — | — |
| HS6M1-4P*1 | 29.17 | A/G | 3131088 | 0.732 | 0.398 | — | — | — |
| OR2J3*1 | 29.25 | C/T | 3129157 | 0.580 | 0.796 | — | — | — |
| OR2J2*1 | 29.25 | A/G | 3130743 | 0.948 | 0.411 | — | — | — |
| OR5V1*1 | 29.43 | G/T | 6930033 | 0.075 | 0.414 | 0.482 | — | — |
| OR5V1*2 | 29.43 | C/T | 4713210 | 0.0013 | 0.763 | — | — | — |
| OR12D3*3 | 29.44 | C/T | 4713211 | 0.873 | 0.369 | — | — | — |
| OR12D3*2 | 29.44 | C/T | 2354502 | 0.185 | 0.612 | — | — | — |
| OR12D3*1 | 29.46 | A/G | 238881 | 0.273 | 0.180 | — | — | — |
| OR12D2*3 | 29.47 | G/T (V47F) | | 0.0090 | 0.670 | 0.054 | — | — |
| OR12D2*2 | 29.47 | C/T (L56P) | 4987411 | 0.183 | 0.835 | 0.108 | — | — |
| OR12D2*1 | 29.47 | A/G (V159I) | 2073151 | 0.0620 | 0.117 | 0.056 | 0.80 | 0.530 |
| OR10C1*1 | 29.52 | C/T | 2074469 | 0.0180 | 0.564 | 0.896 | — | — |
| OR10C1*2 | 29.52 | C/T | 2074464 | 0.771 | 0.336 | 0.182 | — | — |
| OR2H1*1 | 29.54 | C/T | 2021729 | 0.290 | 0.827 | — | — | — |
| OR2H1*2 | 29.54 | C/T | 3128854 | 0.913 | 0.433 | — | — | — |
| MRG*1 | 29.56 | A/G | 1233492 | 0.132 | 0.683 | — | — | — |
| UBD*1 | 29.63 | C/G | 444013 | 0.943 | 0.493 | — | — | — |
| OR2H3*1 | 29.66 | C/T (F587L) | 3129034 | 0.536 | — | 0.332 | — | — |
| OR2H3*2 | 29.66 | C/T (A642V) | 1233387 | 0.424 | — | 0.750 | — | — |
| GABBR1*8 | 29.68 | C/T | | 0.042 | 0.480 | — | — | — |
| GABBR1*7 | 29.68 | C/T | | 0.271 | 0.655 | — | — | — |
| GABBR1*1 | 29.68 | A/G | 881284 | 0.130 | 0.527 | — | — | — |
| GABBR1*19 | 29.68 | A/G | | 0.270 | 0.317 | — | — | — |
| GABBR1*6 | 29.68 | T/C | 29261 | 0.067 | 0.705 | — | — | — |
| GABBR1*5 | 29.68 | T/C | | 0.842 | 0.317 | — | — | — |
| GABBR1*2 | 29.68 | T/C (F658F) | 29230 | 0.018 | 0.617 | 0.352 | 0.30 | 0.470 |
| GABBR1*21 | 29.69 | C/T | | 0.037 | 1 | — | — | — |
| GABBR1*22 | 29.69 | T/C | 29257 | 0.168 | 0.206 | — | — | — |
| GABBR1*4 | 29.69 | ΔA | | 0.462 | — | — | — | — |
| GABBR1*3 | 29.70 | C/G | 29221 | 0.611 | 0.670 | — | — | — |
| GABBR1*24 | 29.70 | C/T | 29242 | 0.296 | 0.257 | — | — | — |
| GABBR1*23 | 29.71 | C/T | | 0.019 | 1 | — | — | — |
| MOG*3 | 29.74 | A/G | 3130250 | 0.466 | 0.655 | — | — | — |
| MOG*2 | 29.74 | A/G | 1318631 | 0.083 | 0.18 | 0.159 | 0.027 | 0.527 |
| MOG*4 | 29.74 | T/G | 2535246 | 0.154 | 0.028 | 0.833 | — | — |
| HLA-F*2 | 29.80 | C/T | 136126 | 0.450 | 0.086 | 0.145 | — | — |
| HLA-F*1 | 29.80 | A/C | 176925 | 0.635 | 0.617 | — | — | — |
| HCGIV.9*1 | 29.87 | C/T | 1610718 | 0.176 | 0.827 | — | — | — |
| HLA-G*−1306 | 29.90 | G/A | 1736936 | 0.065 | 0.144 | 0.046 | — | — |
| HLA-G*−964 | 29.90 | G/A | 1632947 | 0.053 | 0.040 | 0.046 | 0.0040 | 0.151 |
| HLA-G*−725 | 29.90 | C/G/T | 12233334 | 0.596 | 0.317 | 0.60 | 0.62 | 0.060 |
| HLA-G*−689 | 29.90 | A/G | 2735022 | 0.025 | 0.105 | 0.076 | — | — |
| HLA-G*−666 | 29.90 | G/T | 1632945 | 0.121 | 0.170 | 0.076 | — | — |
| HLA-G*−201 | 29.90 | G/A | 1631950 | 0.105 | 0.034 | 0.046 | — | — |
| HLA-G*1489 | 29.90 | C/T (H93H) | 1624278 | 0.182 | 1.0 | 0.51 | — | — |
| HLA-G*1538 | 29.90 | C/T (L110I) | | 0.495 | 1.0 | 0.0030 | — | — |
| HLA- | 29.90 | 14 bp ins/del | 16375 | 0.013 | 0.647 | 0.16 | 0.150 | 0.720 |
| G*3741 ins | | | | | | | | |

TABLE 7
The configuration of 28 SNPs (FIG. 1) on chromosomes (called haplotypes).

| | Frequency | | | | | | | | | | | | | |
|---|---|---|---|---|---|---|---|---|---|---|---|---|---|---|
| | | Eur. | Han | | | | | | | | | | | |
| Associated | Afr. Amer. | Amer. | Chinese | SNPs in the Promoter Region of HLA-G | | | | | | | | | | |
| HLA-G Allele | (N = 44) | (N = 47) | (N = 43) | −1306 | −1179 | −1155 | −1140 | −1138 | −1121 | −990 | −964 | −922* | −810 | −762 |
| 010101a | 0.227 | 0.34 | 0.37 | G | A | G | A | A | C | G | G | C | C | C |
| 010101d | 0.057 | 0.021 | 0 | G | A | G | A | A | C | G | G | C | C | C |
| 010101b | 0.068 | 0.064 | 0.035 | G | A | G | A | A | C | G | G | C | C | C |
| 010101c | 0 | 0.053 | 0.011 | G | A | G | A | A | T | G | G | C | C | C |
| 010101e | 0 | 0 | 0.011 | G | A | G | A | A | C | A | G | C | C | C |
| 010301a | 0.023 | 0.011 | 0.011 | G | G | G | A | G | C | G | G | C | C | C |
| 010301b | 0.011 | 0.011 | 0 | G | G | G | A | G | C | G | G | C | C | C |
| 010301c | 0.034 | 0 | 0 | G | G | G | A | G | C | G | G | C | C | C |
| 010301d | 0.034 | 0 | 0 | G | G | G | A | G | C | G | G | C | C | C |
| 010102a | 0.307 | 0.436 | 0.3 | A | G | G | T | A | C | G | A | C | C | T |
| 010102b | 0 | 0.011 | 0 | A | G | G | T | A | C | G | A | C | T | T |
| 010102c* | 0 | 0 | 0 | A | G | G | T | A | C | G | A | A | C | T |
| 010401a | 0.239 | 0.053 | 0.17 | A | G | A | A | A | C | G | A | C | C | T |
| 010401b | 0 | 0 | 0.08 | A | G | A | A | A | C | G | A | C | C | T |
| orang | | | | A | A | — | A | A | C | G | A | C | T | T |
| chimp | | | | A | G | G | A | A | C | G | A | C | C | T |
| gorilla | | | | A | G | G | A | A | C | G | A | C | T | T |

| Associated | SNPs in the Promoter Region of HLA-G | | | | | | | | | | | | | | | | |
|---|---|---|---|---|---|---|---|---|---|---|---|---|---|---|---|---|---|
| HLA-G Allele | −725 | −716 | −689 | −666 | −646 | −633 | −509 | −486 | −483 | −477 | −443 | −400 | −391 | −369 | −201 | −56 | +15 |
| 010101a | C | T | A | G | A | G | C | A | A | C | G | G | G | C | G | C | G |
| 010101d | C | T | A | G | A | G | C | A | G | C | G | G | G | C | G | C | G |
| 010101b | G | T | A | G | A | G | C | A | A | C | G | G | G | C | G | C | G |
| 010101c | G | T | A | G | A | G | C | A | A | C | G | G | G | C | G | C | G |
| 010101e | C | T | A | G | A | G | C | A | A | C | G | G | G | C | G | C | G |
| 010301a | T | T | A | G | A | G | G | A | A | G | G | A | A | A | G | T | G |
| 010301b | T | T | A | G | G | G | G | A | A | G | G | A | A | A | G | T | G |
| 010301c | T | T | A | G | G | G | G | A | A | G | G | G | G | A | G | T | G |
| 010301d | T | T | A | G | A | G | C | A | A | G | G | A | A | A | G | T | G |
| 010102a | C | G | G | T | A | A | C | C | A | G | G | G | G | A | A | C | A |
| 010102b | C | G | G | T | A | A | C | C | A | G | G | G | G | A | A | C | A |
| 010102c* | C | G | G | T | A | A | C | C | A | G | G | G | G | A | A | C | A |
| 010401a | C | G | G | T | A | A | C | C | A | G | G | G | G | A | A | C | A |
| 010401b | C | G | G | T | A | A | C | C | A | G | A | G | G | A | A | C | A |
| orang | C | T | G | G | A | A | C | C | A | G | G | G | G | A | A | C | G |
| chimp | C | T | G | G | A | A | C | C | A | G | G | G | G | C | QA | C | G |
| gorilla | C | T | G | G | A | A | C | C | A | G | G | G | G | A | A | C | G |

*−922A and the *01012c haplotype was only present in 2 individuals with asthma

TABLE 8
Individual genotype at the HLA-G locus

| Allele1 | Allele2 | −1306A/G | −1179G/A | −1155G/A | −1140T/A | −1138A/G | −1121C/T | −990G/A | −0964A/G | −922C/A | −810C/T |
|---|---|---|---|---|---|---|---|---|---|---|---|
| 010103 | 010103 | 2 | 2 | 2 | 2 | 2 | 2 | 2 | 2 | 2 | 2 |
| 010103 | 010104 | 1 | 1 | 2 | 1 | 2 | 2 | 1 | 1 | 2 | 2 |
| 010103 | 010108 | 1 | 1 | 2 | 1 | 2 | 2 | 1 | 1 | 2 | 2 |
| 010103 | 010101a | 1 | 1 | 2 | 1 | 2 | 2 | 2 | 1 | 2 | 2 |
| 010103 | 010101b | 1 | 1 | 2 | 1 | 2 | 2 | 2 | 1 | 2 | 2 |
| 010103 | 010101c | 1 | 1 | 2 | 1 | 2 | 1 | 2 | 1 | 2 | 2 |
| 010103 | 010101d | 1 | 1 | 2 | 1 | 2 | 2 | 2 | 1 | 2 | 2 |
| 010103 | 010101e | 1 | 1 | 2 | 1 | 2 | 2 | 1 | 1 | 2 | 2 |
| 010103 | 010301a | 1 | 2 | 2 | 1 | 1 | 1 | 2 | 1 | 2 | 2 |
| 010103 | 010301b | 1 | 2 | 2 | 1 | 1 | 1 | 2 | 1 | 2 | 2 |
| 010103 | 010301c | 1 | 2 | 2 | 1 | 1 | 1 | 2 | 1 | 2 | 2 |
| 010103 | 010301d | 1 | 2 | 2 | 1 | 1 | 1 | 2 | 1 | 2 | 2 |
| 010103 | 010401a | 2 | 2 | 1 | 1 | 2 | 2 | 2 | 2 | 2 | 2 |
| 010103 | 010401b | 2 | 2 | 1 | 1 | 2 | 2 | 2 | 2 | 2 | 2 |
| 010108 | 010104 | 0 | 0 | 2 | 0 | 2 | 2 | 0 | 0 | 2 | 2 |
| 010108 | 010108 | 0 | 0 | 2 | 0 | 2 | 2 | 0 | 0 | 2 | 2 |
| 010601 | 010103 | 2 | 2 | 2 | 2 | 2 | 2 | 2 | 2 | 2 | 2 |
| 010601 | 010104 | 1 | 1 | 2 | 1 | 2 | 2 | 1 | 1 | 2 | 2 |
| 010601 | 010108 | 1 | 1 | 2 | 1 | 2 | 2 | 1 | 1 | 2 | 2 |
| 010601 | 010601 | 2 | 2 | 2 | 2 | 2 | 2 | 2 | 2 | 2 | 2 |
| 010601 | 010101a | 1 | 1 | 2 | 1 | 2 | 2 | 2 | 1 | 2 | 2 |
| 010601 | 010101b | 1 | 1 | 2 | 1 | 2 | 2 | 2 | 1 | 2 | 2 |
| 010601 | 010101c | 1 | 1 | 2 | 1 | 2 | 1 | 2 | 1 | 2 | 2 |
| 010601 | 010101d | 1 | 1 | 2 | 1 | 2 | 2 | 2 | 1 | 2 | 2 |
| 010601 | 010101e | 1 | 1 | 2 | 1 | 2 | 2 | 1 | 1 | 2 | 2 |
| 010601 | 010301a | 1 | 2 | 2 | 1 | 1 | 1 | 2 | 1 | 2 | 2 |
| 010601 | 010301b | 1 | 2 | 2 | 1 | 1 | 1 | 2 | 1 | 2 | 2 |
| 010601 | 010301c | 1 | 2 | 2 | 1 | 1 | 1 | 2 | 1 | 2 | 2 |
| 010601 | 010301d | 1 | 2 | 2 | 1 | 1 | 1 | 2 | 1 | 2 | 2 |
| 010601 | 010401a | 2 | 2 | 1 | 1 | 2 | 2 | 2 | 2 | 2 | 2 |
| 010601 | 010401b | 2 | 2 | 1 | 1 | 2 | 2 | 2 | 2 | 2 | 2 |

| Allele1 | Allele2 | −762T/C | −725C | −725G | −725T | −716G/T | −689G/A | −666T/G | −649A/G | −633A/G | −509C/G | −486C/A |
|---|---|---|---|---|---|---|---|---|---|---|---|---|
| 010103 | 010103 | 2 | 2 | 0 | 0 | 2 | 2 | 2 | 2 | 2 | 2 | 2 |
| 010103 | 010104 | 1 | 2 | 0 | 0 | 1 | 1 | 1 | 2 | 1 | 2 | 1 |
| 010103 | 010108 | 1 | 2 | 0 | 0 | 1 | 1 | 1 | 2 | 1 | 2 | 1 |
| 010103 | 010101a | 1 | 2 | 0 | 0 | 1 | 1 | 1 | 2 | 1 | 2 | 1 |
| 010103 | 010101b | 1 | 1 | 1 | 0 | 1 | 1 | 1 | 2 | 1 | 2 | 1 |
| 010103 | 010101c | 1 | 1 | 1 | 0 | 1 | 1 | 1 | 2 | 1 | 2 | 1 |
| 010103 | 010101d | 1 | 2 | 0 | 0 | 1 | 1 | 1 | 2 | 1 | 2 | 1 |
| 010103 | 010101e | 1 | 2 | 0 | 0 | 1 | 1 | 1 | 2 | 1 | 2 | 1 |
| 010103 | 010301a | 1 | 1 | 0 | 1 | 1 | 1 | 1 | 2 | 1 | 1 | 1 |
| 010103 | 010301b | 1 | 1 | 0 | 1 | 1 | 1 | 1 | 1 | 1 | 1 | 1 |
| 010103 | 010301c | 1 | 1 | 0 | 1 | 1 | 1 | 1 | 1 | 1 | 1 | 1 |
| 010103 | 010301d | 1 | 1 | 0 | 1 | 1 | 1 | 1 | 2 | 1 | 2 | 1 |
| 010103 | 010401a | 2 | 2 | 0 | 0 | 2 | 2 | 2 | 2 | 2 | 2 | 2 |
| 010103 | 010401b | 2 | 2 | 0 | 0 | 2 | 2 | 2 | 2 | 2 | 2 | 2 |
| 010108 | 010104 | 0 | 2 | 0 | 0 | 0 | 0 | 0 | 2 | 0 | 2 | 0 |
| 010108 | 010108 | 0 | 2 | 0 | 0 | 0 | 0 | 0 | 2 | 0 | 2 | 0 |
| 010601 | 010103 | 2 | 2 | 0 | 0 | 2 | 2 | 2 | 2 | 2 | 2 | 2 |
| 010601 | 010104 | 1 | 2 | 0 | 0 | 1 | 1 | 1 | 2 | 1 | 2 | 1 |
| 010601 | 010108 | 1 | 2 | 0 | 0 | 1 | 1 | 1 | 2 | 1 | 2 | 1 |
| 010601 | 010601 | 2 | 2 | 0 | 0 | 2 | 2 | 2 | 2 | 2 | 2 | 2 |
| 010601 | 010101a | 1 | 2 | 0 | 0 | 1 | 1 | 1 | 2 | 1 | 2 | 1 |
| 010601 | 010101b | 1 | 1 | 1 | 0 | 1 | 1 | 1 | 2 | 1 | 2 | 1 |
| 010601 | 010101c | 1 | 1 | 1 | 0 | 1 | 1 | 1 | 2 | 1 | 2 | 1 |
| 010601 | 010101d | 1 | 2 | 0 | 0 | 1 | 1 | 1 | 2 | 1 | 2 | 1 |
| 010601 | 010101e | 1 | 2 | 0 | 0 | 1 | 1 | 1 | 2 | 1 | 2 | 1 |
| 010601 | 010301a | 1 | 1 | 0 | 1 | 1 | 1 | 1 | 2 | 1 | 1 | 1 |
| 010601 | 010301b | 1 | 1 | 0 | 1 | 1 | 1 | 1 | 1 | 1 | 1 | 1 |
| 010601 | 010301c | 1 | 1 | 0 | 1 | 1 | 1 | 1 | 1 | 1 | 1 | 1 |
| 010601 | 010301d | 1 | 1 | 0 | 1 | 1 | 1 | 1 | 2 | 1 | 2 | 1 |
| 010601 | 010401a | 2 | 2 | 0 | 0 | 2 | 2 | 2 | 2 | 2 | 2 | 2 |
| 010601 | 010401b | 2 | 2 | 0 | 0 | 2 | 2 | 2 | 2 | 2 | 2 | 2 |

| Allele1 | Allele2 | −482A/G | −477G/C | −443G/A | −400G/A | −391G/A | −369A/G | −201A/G | −56C/T | +15A/G | +36A/G | AA31 (A/T) |
|---|---|---|---|---|---|---|---|---|---|---|---|---|
| 010103 | 010103 | 2 | 2 | 2 | 2 | 2 | 2 | 2 | 2 | 2 | 2 | 2 |
| 010103 | 010104 | 2 | 1 | 2 | 2 | 1 | 1 | 1 | 2 | 1 | 1 | 2 |
| 010103 | 010108 | 2 | 1 | 2 | 2 | 1 | 1 | 1 | 2 | 1 | 1 | 2 |
| 010103 | 010101a | 2 | 1 | 2 | 2 | 1 | 1 | 1 | 2 | 1 | 1 | 2 |
| 010103 | 010101b | 2 | 1 | 2 | 2 | 1 | 1 | 1 | 2 | 1 | 1 | 2 |
| 010103 | 010101c | 2 | 1 | 2 | 2 | 1 | 1 | 1 | 2 | 1 | 1 | 2 |
| 010103 | 010101d | 1 | 1 | 2 | 2 | 1 | 1 | 1 | 2 | 1 | 1 | 2 |
| 010103 | 010101e | 2 | 1 | 2 | 2 | 1 | 1 | 1 | 2 | 1 | 1 | 2 |
| 010103 | 010301a | 2 | 2 | 2 | 2 | 1 | 1 | 1 | 1 | 1 | 1 | 1 |
| 010103 | 010301b | 2 | 2 | 2 | 1 | 1 | 2 | 1 | 1 | 1 | 1 | 1 |
| 010103 | 010301c | 2 | 2 | 2 | 2 | 2 | 2 | 1 | 1 | 1 | 1 | 1 |
| 010103 | 010301d | 2 | 2 | 2 | 1 | 1 | 2 | 1 | 1 | 1 | 1 | 1 |
| 010103 | 010401a | 2 | 2 | 2 | 2 | 2 | 2 | 2 | 2 | 2 | 2 | 2 |
| 010103 | 010401b | 2 | 2 | 1 | 2 | 2 | 2 | 2 | 2 | 2 | 2 | 2 |
| 010108 | 010104 | 2 | 0 | 2 | 2 | 0 | 0 | 0 | 2 | 0 | 0 | 2 |
| 010108 | 010108 | 2 | 0 | 2 | 2 | 0 | 0 | 0 | 2 | 0 | 0 | 2 |
| 010601 | 010103 | 2 | 2 | 2 | 2 | 2 | 2 | 2 | 2 | 2 | 2 | 2 |
| 010601 | 010104 | 2 | 1 | 2 | 2 | 1 | 1 | 1 | 2 | 1 | 1 | 2 |
| 010601 | 010108 | 2 | 1 | 2 | 2 | 1 | 1 | 1 | 2 | 1 | 1 | 2 |
| 010601 | 010601 | 2 | 2 | 2 | 2 | 2 | 2 | 2 | 2 | 2 | 2 | 2 |
| 010601 | 010101a | 2 | 1 | 2 | 2 | 1 | 1 | 1 | 2 | 1 | 1 | 2 |
| 010601 | 010101b | 2 | 1 | 2 | 2 | 1 | 1 | 1 | 2 | 1 | 1 | 2 |
| 010601 | 010101c | 2 | 1 | 2 | 2 | 1 | 1 | 1 | 2 | 1 | 1 | 2 |
| 010601 | 010101d | 1 | 1 | 2 | 2 | 1 | 1 | 1 | 2 | 1 | 1 | 2 |
| 010601 | 010101e | 2 | 1 | 2 | 2 | 1 | 1 | 1 | 2 | 1 | 1 | 2 |
| 010601 | 010301a | 2 | 2 | 2 | 2 | 1 | 1 | 1 | 1 | 1 | 1 | 1 |
| 010601 | 010301b | 2 | 2 | 2 | 1 | 1 | 2 | 1 | 1 | 1 | 1 | 1 |
| 010601 | 010301c | 2 | 2 | 2 | 2 | 2 | 2 | 1 | 1 | 1 | 1 | 1 |
| 010601 | 010301d | 2 | 2 | 2 | 1 | 1 | 2 | 1 | 1 | 1 | 1 | 1 |
| 010601 | 010401a | 2 | 2 | 2 | 2 | 2 | 2 | 2 | 2 | 2 | 2 | 2 |
| 010601 | 010401b | 2 | 2 | 1 | 2 | 2 | 2 | 2 | 2 | 2 | 2 | 2 |

| | | AA35 | AA57 | AA69 | AA93 | AA100 | AA107 | AA110 | AA130 | | | |
|---|---|---|---|---|---|---|---|---|---|---|---|---|
| Allele1 | Allele2 | (A/T) | (A/G) | (C/T) | (T/C) | (C/T) | (A/T) | (C/A) | (C/D) | AA188 (C/T) | AA258 (C/T) | 14 bp in/del |
| 010103 | 010103 | 2 | 2 | 2 | 0 | 2 | 0 | 2 | 2 | 2 | 2 | 2 |
| 010103 | 010104 | 2 | 2 | 1 | 0 | 2 | 1 | 2 | 2 | 2 | 2 | 1 |
| 010103 | 010108 | 2 | 2 | 2 | 0 | 2 | 1 | 2 | 2 | 2 | 2 | 1 |
| 010103 | 010101a | 2 | 1 | 2 | 0 | 2 | 1 | 2 | 2 | 2 | 2 | 1 |
| 010103 | 010101b | 2 | 1 | 2 | 0 | 2 | 1 | 2 | 2 | 2 | 2 | 1 |
| 010103 | 010101c | 2 | 1 | 2 | 0 | 2 | 1 | 2 | 2 | 2 | 2 | 1 |
| 010103 | 010101d | 2 | 1 | 2 | 0 | 2 | 1 | 2 | 2 | 2 | 2 | 1 |
| 010103 | 010101e | 2 | 1 | 2 | 0 | 2 | 1 | 2 | 2 | 2 | 2 | 1 |
| 010103 | 010301a | 2 | 1 | 2 | 0 | 2 | 1 | 2 | 2 | 1 | 2 | 2 |
| 010103 | 010301b | 2 | 1 | 2 | 0 | 2 | 1 | 2 | 2 | 1 | 2 | 2 |
| 010103 | 010301c | 2 | 1 | 2 | 0 | 2 | 1 | 2 | 2 | 1 | 2 | 2 |
| 010103 | 010301d | 2 | 1 | 2 | 0 | 2 | 1 | 2 | 2 | 1 | 2 | 2 |
| 010103 | 010401a | 2 | 2 | 2 | 0 | 2 | 1 | 1 | 2 | 2 | 2 | 1 |
| 010103 | 010401b | 2 | 2 | 2 | 0 | 2 | 1 | 1 | 2 | 2 | 2 | 1 |
| 010108 | 010104 | 2 | 2 | 1 | 0 | 2 | 2 | 2 | 2 | 2 | 2 | 0 |
| 010108 | 010108 | 2 | 2 | 2 | 0 | 2 | 2 | 2 | 2 | 2 | 2 | 0 |
| 010601 | 010103 | 2 | 2 | 2 | 1 | 2 | 1 | 2 | 2 | 2 | 1 | 2 |
| 010601 | 010104 | 2 | 2 | 1 | 1 | 2 | 2 | 2 | 2 | 2 | 1 | 1 |
| 010601 | 010108 | 2 | 2 | 2 | 1 | 2 | 2 | 2 | 2 | 2 | 1 | 1 |
| 010601 | 010601 | 2 | 2 | 2 | 2 | 2 | 2 | 2 | 2 | 2 | 0 | 2 |
| 010601 | 010101a | 2 | 1 | 2 | 1 | 2 | 2 | 2 | 2 | 2 | 1 | 1 |
| 010601 | 010101b | 2 | 1 | 2 | 1 | 2 | 2 | 2 | 2 | 2 | 1 | 1 |
| 010601 | 010101c | 2 | 1 | 2 | 1 | 2 | 2 | 2 | 2 | 2 | 1 | 1 |
| 010601 | 010101d | 2 | 1 | 2 | 1 | 2 | 2 | 2 | 2 | 2 | 1 | 1 |
| 010601 | 010101e | 2 | 1 | 2 | 1 | 2 | 2 | 2 | 2 | 2 | 1 | 1 |
| 010601 | 010301a | 2 | 1 | 2 | 1 | 2 | 2 | 2 | 2 | 1 | 1 | 2 |
| 010601 | 010301b | 2 | 1 | 2 | 1 | 2 | 2 | 2 | 2 | 1 | 1 | 2 |
| 010601 | 010301c | 2 | 1 | 2 | 1 | 2 | 2 | 2 | 2 | 1 | 1 | 2 |
| 010601 | 010301d | 2 | 1 | 2 | 1 | 2 | 2 | 2 | 2 | 1 | 1 | 2 |
| 010601 | 010401a | 2 | 2 | 2 | 1 | 2 | 2 | 1 | 2 | 2 | 1 | 1 |
| 010601 | 010401b | 2 | 2 | 2 | 1 | 2 | 2 | 1 | 2 | 2 | 1 | 1 |

| Allele1 | Allele2 | −1306A/G | −1179G/A | −1155G/A | −1140T/A | −1138A/G | −1121C/T | −990G/A | −0964A/G | −922C/A | −810C/T |
|---|---|---|---|---|---|---|---|---|---|---|---|
| 010601 | 0105N | 2 | 2 | 2 | 2 | 2 | 2 | 2 | 2 | 2 | 2 |
| 010101a | 010104 | 0 | 0 | 2 | 0 | 2 | 2 | 1 | 0 | 2 | 2 |
| 010101a | 010108 | 0 | 0 | 2 | 0 | 2 | 2 | 1 | 0 | 2 | 2 |
| 010101a | 010101a | 0 | 0 | 2 | 0 | 2 | 2 | 2 | 0 | 2 | 2 |
| 010101a | 010101b | 0 | 0 | 2 | 0 | 2 | 2 | 2 | 0 | 2 | 2 |
| 010101a | 010101c | 0 | 0 | 2 | 0 | 2 | 1 | 2 | 0 | 2 | 2 |
| 010101a | 010101d | 0 | 0 | 2 | 0 | 2 | 2 | 2 | 0 | 2 | 2 |
| 010101a | 010101e | 0 | 0 | 2 | 0 | 2 | 2 | 1 | 0 | 2 | 2 |
| 010101b | 010104 | 0 | 0 | 2 | 0 | 2 | 2 | 1 | 0 | 2 | 2 |
| 010101b | 010108 | 0 | 0 | 2 | 0 | 2 | 2 | 1 | 0 | 2 | 2 |
| 010101b | 010101b | 0 | 0 | 2 | 0 | 2 | 2 | 2 | 0 | 2 | 2 |
| 010101b | 010101c | 0 | 0 | 2 | 0 | 2 | 1 | 2 | 0 | 2 | 2 |
| 010101b | 010101d | 0 | 0 | 2 | 0 | 2 | 2 | 2 | 0 | 2 | 2 |
| 010101b | 010101e | 0 | 0 | 2 | 0 | 2 | 2 | 1 | 0 | 2 | 2 |
| 010101c | 010104 | 0 | 0 | 2 | 0 | 2 | 1 | 1 | 0 | 2 | 2 |
| 010101c | 010108 | 0 | 0 | 2 | 0 | 2 | 1 | 1 | 0 | 2 | 2 |
| 010101c | 010101c | 0 | 0 | 2 | 0 | 2 | 0 | 2 | 0 | 2 | 2 |
| 010101c | 010101d | 0 | 0 | 2 | 0 | 2 | 1 | 2 | 0 | 2 | 2 |
| 010101c | 010101e | 0 | 0 | 2 | 0 | 2 | 1 | 1 | 0 | 2 | 2 |
| 010101d | 010104 | 0 | 0 | 2 | 0 | 2 | 2 | 1 | 0 | 2 | 2 |
| 010101d | 010108 | 0 | 0 | 2 | 0 | 2 | 2 | 1 | 0 | 2 | 2 |
| 010101d | 010101d | 0 | 0 | 2 | 0 | 2 | 2 | 2 | 0 | 2 | 2 |
| 010101d | 010101e | 0 | 0 | 2 | 0 | 2 | 2 | 1 | 0 | 2 | 2 |
| 010101e | 010104 | 0 | 0 | 2 | 0 | 2 | 2 | 0 | 0 | 2 | 2 |
| 010101e | 010108 | 0 | 0 | 2 | 0 | 2 | 2 | 0 | 0 | 2 | 2 |
| 010101e | 010101e | 0 | 0 | 2 | 0 | 2 | 2 | 0 | 0 | 2 | 2 |
| 010102a | 010103 | 2 | 2 | 2 | 2 | 2 | 2 | 2 | 2 | 2 | 2 |
| 010102a | 010104 | 1 | 1 | 2 | 1 | 2 | 2 | 1 | 1 | 2 | 2 |
| 010102a | 010108 | 1 | 1 | 2 | 1 | 2 | 2 | 1 | 1 | 2 | 2 |
| 010102a | 010601 | 2 | 2 | 2 | 2 | 2 | 2 | 2 | 2 | 2 | 2 |
| 010102a | 010101a | 1 | 1 | 2 | 1 | 2 | 2 | 2 | 1 | 2 | 2 |

| Allele1 | Allele2 | −762T/C | −725C | −725G | −725T | −716G/T | −689G/A | −666T/G | −649A/G | −633A/G | −509C/G | −486C/A |
|---|---|---|---|---|---|---|---|---|---|---|---|---|
| 010601 | 0105N | 2 | 2 | 0 | 0 | 2 | 2 | 2 | 2 | 2 | 2 | 2 |
| 010101a | 010104 | 0 | 2 | 0 | 0 | 0 | 0 | 0 | 2 | 0 | 2 | 0 |
| 010101a | 010108 | 0 | 2 | 0 | 0 | 0 | 0 | 0 | 2 | 0 | 2 | 0 |
| 010101a | 010101a | 0 | 2 | 0 | 0 | 0 | 0 | 0 | 2 | 0 | 2 | 0 |
| 010101a | 010101b | 0 | 1 | 1 | 0 | 0 | 0 | 0 | 2 | 0 | 2 | 0 |
| 010101a | 010101c | 0 | 1 | 1 | 0 | 0 | 0 | 0 | 2 | 0 | 2 | 0 |
| 010101a | 010101d | 0 | 2 | 0 | 0 | 0 | 0 | 0 | 2 | 0 | 2 | 0 |
| 010101a | 010101e | 0 | 2 | 0 | 0 | 0 | 0 | 0 | 2 | 0 | 2 | 0 |
| 010101b | 010104 | 0 | 1 | 1 | 0 | 0 | 0 | 0 | 2 | 0 | 2 | 0 |
| 010101b | 010108 | 0 | 1 | 1 | 0 | 0 | 0 | 0 | 2 | 0 | 2 | 0 |
| 010101b | 010101b | 0 | 0 | 2 | 0 | 0 | 0 | 0 | 2 | 0 | 2 | 0 |
| 010101b | 010101c | 0 | 0 | 2 | 0 | 0 | 0 | 0 | 2 | 0 | 2 | 0 |
| 010101b | 010101d | 0 | 1 | 1 | 0 | 0 | 0 | 0 | 2 | 0 | 2 | 0 |
| 010101b | 010101e | 0 | 1 | 1 | 0 | 0 | 0 | 0 | 2 | 0 | 2 | 0 |
| 010101c | 010104 | 0 | 1 | 1 | 0 | 0 | 0 | 0 | 2 | 0 | 2 | 0 |
| 010101c | 010108 | 0 | 1 | 1 | 0 | 0 | 0 | 0 | 2 | 0 | 2 | 0 |
| 010101c | 010101c | 0 | 0 | 2 | 0 | 0 | 0 | 0 | 2 | 0 | 2 | 0 |
| 010101c | 010101d | 0 | 1 | 1 | 0 | 0 | 0 | 0 | 2 | 0 | 2 | 0 |
| 010101c | 010101e | 0 | 1 | 1 | 0 | 0 | 0 | 0 | 2 | 0 | 2 | 0 |
| 010101d | 010104 | 0 | 2 | 0 | 0 | 0 | 0 | 0 | 2 | 0 | 2 | 0 |
| 010101d | 010108 | 0 | 2 | 0 | 0 | 0 | 0 | 0 | 2 | 0 | 2 | 0 |
| 010101d | 010101d | 0 | 2 | 0 | 0 | 0 | 0 | 0 | 2 | 0 | 2 | 0 |
| 010101d | 010101e | 0 | 2 | 0 | 0 | 0 | 0 | 0 | 2 | 0 | 2 | 0 |
| 010101e | 010104 | 0 | 2 | 0 | 0 | 0 | 0 | 0 | 2 | 0 | 2 | 0 |
| 010101e | 010108 | 0 | 2 | 0 | 0 | 0 | 0 | 0 | 2 | 0 | 2 | 0 |
| 010101e | 010101e | 0 | 2 | 0 | 0 | 0 | 0 | 0 | 2 | 0 | 2 | 0 |
| 010102a | 010103 | 2 | 2 | 0 | 0 | 2 | 2 | 2 | 2 | 2 | 2 | 2 |
| 010102a | 010104 | 1 | 2 | 0 | 0 | 1 | 1 | 1 | 2 | 1 | 2 | 1 |
| 010102a | 010108 | 1 | 2 | 0 | 0 | 1 | 1 | 1 | 2 | 1 | 2 | 1 |
| 010102a | 010601 | 2 | 2 | 0 | 0 | 2 | 2 | 2 | 2 | 2 | 2 | 2 |
| 010102a | 010101a | 1 | 2 | 0 | 0 | 1 | 1 | 1 | 2 | 1 | 2 | 1 |

| Allele1 | Allele2 | −482A/G | −477G/C | −443G/A | −400G/A | −391G/A | −369A/G | −201A/G | −56C/T | +15A/G | +36A/G | AA31 (A/T) |
|---|---|---|---|---|---|---|---|---|---|---|---|---|
| 010601 | 0105N | 2 | 2 | 2 | 2 | 2 | 2 | 2 | 2 | 2 | 2 | 2 |
| 010101a | 010104 | 2 | 0 | 2 | 2 | 0 | 0 | 0 | 2 | 0 | 0 | 2 |
| 010101a | 010108 | 2 | 0 | 2 | 2 | 0 | 0 | 0 | 2 | 0 | 0 | 2 |
| 010101a | 010101a | 2 | 0 | 2 | 2 | 0 | 0 | 0 | 2 | 0 | 0 | 2 |
| 010101a | 010101b | 2 | 0 | 2 | 2 | 0 | 0 | 0 | 2 | 0 | 0 | 2 |
| 010101a | 010101c | 2 | 0 | 2 | 2 | 0 | 0 | 0 | 2 | 0 | 0 | 2 |
| 010101a | 010101d | 1 | 0 | 2 | 2 | 0 | 0 | 0 | 2 | 0 | 0 | 2 |
| 010101a | 010101e | 2 | 0 | 2 | 2 | 0 | 0 | 0 | 2 | 0 | 0 | 2 |
| 010101b | 010104 | 2 | 0 | 2 | 2 | 0 | 0 | 0 | 2 | 0 | 0 | 2 |
| 010101b | 010108 | 2 | 0 | 2 | 2 | 0 | 0 | 0 | 2 | 0 | 0 | 2 |
| 010101b | 010101b | 2 | 0 | 2 | 2 | 0 | 0 | 0 | 2 | 0 | 0 | 2 |
| 010101b | 010101c | 2 | 0 | 2 | 2 | 0 | 0 | 0 | 2 | 0 | 0 | 2 |
| 010101b | 010101d | 1 | 0 | 2 | 2 | 0 | 0 | 0 | 2 | 0 | 0 | 2 |
| 010101b | 010101e | 2 | 0 | 2 | 2 | 0 | 0 | 0 | 2 | 0 | 0 | 2 |
| 010101c | 010104 | 2 | 0 | 2 | 2 | 0 | 0 | 0 | 2 | 0 | 0 | 2 |
| 010101c | 010108 | 2 | 0 | 2 | 2 | 0 | 0 | 0 | 2 | 0 | 0 | 2 |
| 010101c | 010101c | 2 | 0 | 2 | 2 | 0 | 0 | 0 | 2 | 0 | 0 | 2 |
| 010101c | 010101d | 1 | 0 | 2 | 2 | 0 | 0 | 0 | 2 | 0 | 0 | 2 |
| 010101c | 010101e | 2 | 0 | 2 | 2 | 0 | 0 | 0 | 2 | 0 | 0 | 2 |
| 010101d | 010104 | 1 | 0 | 2 | 2 | 0 | 0 | 0 | 2 | 0 | 0 | 2 |
| 010101d | 010108 | 1 | 0 | 2 | 2 | 0 | 0 | 0 | 2 | 0 | 0 | 2 |
| 010101d | 010101d | 0 | 0 | 2 | 2 | 0 | 0 | 0 | 2 | 0 | 0 | 2 |
| 010101d | 010101e | 1 | 0 | 2 | 2 | 0 | 0 | 0 | 2 | 0 | 0 | 2 |
| 010101e | 010104 | 2 | 0 | 2 | 2 | 0 | 0 | 0 | 2 | 0 | 0 | 2 |
| 010101e | 010108 | 2 | 0 | 2 | 2 | 0 | 0 | 0 | 2 | 0 | 0 | 2 |
| 010101e | 010101e | 2 | 0 | 2 | 2 | 0 | 0 | 0 | 2 | 0 | 0 | 2 |
| 010102a | 010103 | 2 | 2 | 2 | 2 | 2 | 2 | 2 | 2 | 2 | 2 | 2 |
| 010102a | 010104 | 2 | 1 | 2 | 2 | 1 | 1 | 1 | 2 | 1 | 1 | 2 |
| 010102a | 010108 | 2 | 1 | 2 | 2 | 1 | 1 | 1 | 2 | 1 | 1 | 2 |
| 010102a | 010601 | 2 | 2 | 2 | 2 | 2 | 2 | 2 | 2 | 2 | 2 | 2 |
| 010102a | 010101a | 2 | 1 | 2 | 2 | 1 | 1 | 1 | 2 | 1 | 1 | 2 |

| | | AA35 | AA57 | AA69 | AA93 | AA100 | AA107 | AA110 | AA130 | | | |
|---|---|---|---|---|---|---|---|---|---|---|---|---|
| Allele1 | Allele2 | (A/T) | (A/G) | (C/T) | (T/C) | (C/T) | (A/T) | (C/A) | (C/D) | AA188 (C/T) | AA258 (C/T) | 14 bp in/del |
| 010601 | 0105N | 2 | 2 | 2 | 2 | 2 | 2 | 2 | 1 | 2 | 1 | 2 |
| 010101a | 010104 | 2 | 1 | 1 | 0 | 2 | 2 | 2 | 2 | 2 | 2 | 0 |
| 010101a | 010108 | 2 | 1 | 2 | 0 | 2 | 2 | 2 | 2 | 2 | 2 | 0 |
| 010101a | 010101a | 2 | 0 | 2 | 0 | 2 | 2 | 2 | 2 | 2 | 2 | 0 |
| 010101a | 010101b | 2 | 0 | 2 | 0 | 2 | 2 | 2 | 2 | 2 | 2 | 0 |
| 010101a | 010101c | 2 | 0 | 2 | 0 | 2 | 2 | 2 | 2 | 2 | 2 | 0 |
| 010101a | 010101d | 2 | 0 | 2 | 0 | 2 | 2 | 2 | 2 | 2 | 2 | 0 |
| 010101a | 010101e | 2 | 0 | 2 | 0 | 2 | 2 | 2 | 2 | 2 | 2 | 0 |
| 010101b | 010104 | 2 | 1 | 1 | 0 | 2 | 2 | 2 | 2 | 2 | 2 | 0 |
| 010101b | 010108 | 2 | 1 | 2 | 0 | 2 | 2 | 2 | 2 | 2 | 2 | 0 |
| 010101b | 010101b | 2 | 0 | 2 | 0 | 2 | 2 | 2 | 2 | 2 | 2 | 0 |
| 010101b | 010101c | 2 | 0 | 2 | 0 | 2 | 2 | 2 | 2 | 2 | 2 | 0 |
| 010101b | 010101d | 2 | 0 | 2 | 0 | 2 | 2 | 2 | 2 | 2 | 2 | 0 |
| 010101b | 010101e | 2 | 0 | 2 | 0 | 2 | 2 | 2 | 2 | 2 | 2 | 0 |
| 010101c | 010104 | 2 | 1 | 1 | 0 | 2 | 2 | 2 | 2 | 2 | 2 | 0 |
| 010101c | 010108 | 2 | 1 | 2 | 0 | 2 | 2 | 2 | 2 | 2 | 2 | 0 |
| 010101c | 010101c | 2 | 0 | 2 | 0 | 2 | 2 | 2 | 2 | 2 | 2 | 0 |
| 010101c | 010101d | 2 | 0 | 2 | 0 | 2 | 2 | 2 | 2 | 2 | 2 | 0 |
| 010101c | 010101e | 2 | 0 | 2 | 0 | 2 | 2 | 2 | 2 | 2 | 2 | 0 |
| 010101d | 010104 | 2 | 1 | 1 | 0 | 2 | 2 | 2 | 2 | 2 | 2 | 0 |
| 010101d | 010108 | 2 | 1 | 2 | 0 | 2 | 2 | 2 | 2 | 2 | 2 | 0 |
| 010101d | 010101d | 2 | 0 | 2 | 0 | 2 | 2 | 2 | 2 | 2 | 2 | 0 |
| 010101d | 010101e | 2 | 0 | 2 | 0 | 2 | 2 | 2 | 2 | 2 | 2 | 0 |
| 010101e | 010104 | 2 | 1 | 1 | 0 | 2 | 2 | 2 | 2 | 2 | 2 | 0 |
| 010101e | 010108 | 2 | 1 | 2 | 0 | 2 | 2 | 2 | 2 | 2 | 2 | 0 |
| 010101e | 010101e | 2 | 0 | 2 | 0 | 2 | 2 | 2 | 2 | 2 | 2 | 0 |
| 010101a | 010103 | 2 | 2 | 2 | 1 | 2 | 1 | 2 | 2 | 2 | 2 | 2 |
| 010101a | 010104 | 2 | 2 | 1 | 1 | 2 | 2 | 2 | 2 | 2 | 2 | 1 |
| 010101a | 010108 | 2 | 2 | 2 | 1 | 2 | 2 | 2 | 2 | 2 | 2 | 1 |
| 010101a | 010601 | 2 | 2 | 2 | 2 | 2 | 2 | 2 | 2 | 2 | 1 | 2 |
| 010101a | 010101a | 2 | 1 | 2 | 1 | 2 | 2 | 2 | 2 | 2 | 2 | 1 |

| Allele1 | Allele2 | −1306A/G | −1179G/A | −1155G/A | −1140T/A | −1138A/G | −1121C/T | −990G/A | −0964A/G | −922C/A | −810C/T |
|---|---|---|---|---|---|---|---|---|---|---|---|
| 010102a | 010101b | 1 | 1 | 2 | 1 | 2 | 2 | 2 | 1 | 2 | 2 |
| 010102a | 010101c | 1 | 1 | 2 | 1 | 2 | 1 | 2 | 1 | 2 | 2 |
| 010102a | 010101d | 1 | 1 | 2 | 1 | 2 | 2 | 2 | 1 | 2 | 2 |
| 010102a | 010101e | 1 | 1 | 2 | 1 | 2 | 2 | 1 | 1 | 2 | 2 |
| 010102a | 010102a | 2 | 2 | 2 | 2 | 2 | 2 | 2 | 2 | 2 | 2 |
| 010102a | 010102b | 2 | 2 | 2 | 2 | 2 | 2 | 2 | 2 | 2 | 1 |
| 010102a | 010102c | 2 | 2 | 2 | 2 | 2 | 2 | 2 | 2 | 1 | 2 |
| 010102a | 010301a | 1 | 2 | 2 | 1 | 1 | 1 | 2 | 1 | 2 | 2 |
| 010102a | 010301b | 1 | 2 | 2 | 1 | 1 | 1 | 2 | 1 | 2 | 2 |
| 010102a | 010301c | 1 | 2 | 2 | 1 | 1 | 1 | 2 | 1 | 2 | 2 |
| 010102a | 010301d | 1 | 2 | 2 | 1 | 1 | 1 | 2 | 1 | 2 | 2 |
| 010102a | 010401a | 2 | 2 | 1 | 1 | 2 | 2 | 2 | 2 | 2 | 2 |
| 010102a | 010401b | 2 | 2 | 1 | 1 | 2 | 2 | 2 | 2 | 2 | 2 |
| 010102a | 0105N | 2 | 2 | 2 | 2 | 2 | 2 | 2 | 2 | 2 | 2 |
| 010102b | 010103 | 2 | 2 | 2 | 2 | 2 | 2 | 2 | 2 | 2 | 1 |
| 010102b | 010104 | 1 | 1 | 2 | 1 | 2 | 2 | 1 | 1 | 2 | 1 |
| 010102b | 010108 | 1 | 1 | 2 | 1 | 2 | 2 | 1 | 1 | 2 | 1 |
| 010102b | 010601 | 2 | 2 | 2 | 2 | 2 | 2 | 2 | 2 | 2 | 1 |
| 010102b | 010101a | 1 | 1 | 2 | 1 | 2 | 2 | 2 | 1 | 2 | 1 |
| 010102b | 010101b | 1 | 1 | 2 | 1 | 2 | 2 | 2 | 1 | 2 | 1 |
| 010102b | 010101c | 1 | 1 | 2 | 1 | 2 | 1 | 2 | 1 | 2 | 1 |
| 010102b | 010101d | 1 | 1 | 2 | 1 | 2 | 2 | 2 | 1 | 2 | 1 |
| 010102b | 010101e | 1 | 1 | 2 | 1 | 2 | 2 | 1 | 1 | 2 | 1 |
| 010102b | 010102b | 2 | 2 | 2 | 2 | 2 | 2 | 2 | 2 | 2 | 0 |
| 010102b | 010102c | 2 | 2 | 2 | 2 | 2 | 2 | 2 | 2 | 1 | 1 |
| 010102b | 010301a | 1 | 2 | 2 | 1 | 1 | 1 | 2 | 1 | 2 | 1 |
| 010102b | 010301b | 1 | 2 | 2 | 1 | 1 | 1 | 2 | 1 | 2 | 1 |
| 010102b | 010301c | 1 | 2 | 2 | 1 | 1 | 1 | 2 | 1 | 2 | 1 |
| 010102b | 010301d | 1 | 2 | 2 | 1 | 1 | 1 | 2 | 1 | 2 | 1 |
| 010102b | 010401a | 2 | 2 | 1 | 1 | 2 | 2 | 2 | 2 | 2 | 1 |
| 010102b | 010401b | 2 | 2 | 1 | 1 | 2 | 2 | 2 | 2 | 2 | 1 |

| Allele1 | Allele2 | −762T/C | −725C | −725G | −725T | −716G/T | −689G/A | −666T/G | −649A/G | −633A/G | −509C/G | −486C/A |
|---|---|---|---|---|---|---|---|---|---|---|---|---|
| 010102a | 010101b | 1 | 1 | 1 | 0 | 1 | 1 | 1 | 2 | 1 | 2 | 1 |
| 010102a | 010101c | 1 | 1 | 1 | 0 | 1 | 1 | 1 | 2 | 1 | 2 | 1 |
| 010102a | 010101d | 1 | 2 | 0 | 0 | 1 | 1 | 1 | 2 | 1 | 2 | 1 |
| 010102a | 010101e | 1 | 2 | 0 | 0 | 1 | 1 | 1 | 2 | 1 | 2 | 1 |
| 010102a | 010102a | 2 | 2 | 0 | 0 | 2 | 2 | 2 | 2 | 2 | 2 | 2 |
| 010102a | 010102b | 2 | 2 | 0 | 0 | 2 | 2 | 2 | 2 | 2 | 2 | 2 |
| 010102a | 010102c | 2 | 2 | 0 | 0 | 2 | 2 | 2 | 2 | 2 | 2 | 2 |
| 010102a | 010301a | 1 | 1 | 0 | 1 | 1 | 1 | 1 | 2 | 1 | 1 | 1 |
| 010102a | 010301b | 1 | 1 | 0 | 1 | 1 | 1 | 1 | 1 | 1 | 1 | 1 |
| 010102a | 010301c | 1 | 1 | 0 | 1 | 1 | 1 | 1 | 1 | 1 | 1 | 1 |
| 010102a | 010301d | 1 | 1 | 0 | 1 | 1 | 1 | 1 | 2 | 1 | 2 | 1 |
| 010102a | 010401a | 2 | 2 | 0 | 0 | 2 | 2 | 2 | 2 | 2 | 2 | 2 |
| 010102a | 010401b | 2 | 2 | 0 | 0 | 2 | 2 | 2 | 2 | 2 | 2 | 2 |
| 010102a | 0105N | 2 | 2 | 0 | 0 | 2 | 2 | 2 | 2 | 2 | 2 | 2 |
| 010102b | 010103 | 2 | 2 | 0 | 0 | 2 | 2 | 2 | 2 | 2 | 2 | 2 |
| 010102b | 010104 | 1 | 2 | 0 | 0 | 1 | 1 | 1 | 2 | 1 | 2 | 1 |
| 010102b | 010108 | 1 | 2 | 0 | 0 | 1 | 1 | 1 | 2 | 1 | 2 | 1 |
| 010102b | 010601 | 2 | 2 | 0 | 0 | 2 | 2 | 2 | 2 | 2 | 2 | 2 |
| 010102b | 010101a | 1 | 2 | 0 | 0 | 1 | 1 | 1 | 2 | 1 | 2 | 1 |
| 010102b | 010101b | 1 | 1 | 1 | 0 | 1 | 1 | 1 | 2 | 1 | 2 | 1 |
| 010102b | 010101c | 1 | 1 | 1 | 0 | 1 | 1 | 1 | 2 | 1 | 2 | 1 |
| 010102b | 010101d | 1 | 2 | 0 | 0 | 1 | 1 | 1 | 2 | 1 | 2 | 1 |
| 010102b | 010101e | 1 | 2 | 0 | 0 | 1 | 1 | 1 | 2 | 1 | 2 | 1 |
| 010102b | 010102b | 2 | 2 | 0 | 0 | 2 | 2 | 2 | 2 | 2 | 2 | 2 |
| 010102b | 010102c | 2 | 2 | 0 | 0 | 2 | 2 | 2 | 2 | 2 | 2 | 2 |
| 010102b | 010301a | 1 | 1 | 0 | 1 | 1 | 1 | 1 | 2 | 1 | 1 | 1 |
| 010102b | 010301b | 1 | 1 | 0 | 1 | 1 | 1 | 1 | 1 | 1 | 1 | 1 |
| 010102b | 010301c | 1 | 1 | 0 | 1 | 1 | 1 | 1 | 1 | 1 | 1 | 1 |
| 010102b | 010301d | 1 | 1 | 0 | 1 | 1 | 1 | 1 | 2 | 1 | 2 | 1 |
| 010102b | 010401a | 2 | 2 | 0 | 0 | 2 | 2 | 2 | 2 | 2 | 2 | 2 |
| 010102b | 010401b | 2 | 2 | 0 | 0 | 2 | 2 | 2 | 2 | 2 | 2 | 2 |

| Allele1 | Allele2 | −482A/G | −477G/C | −443G/A | −400G/A | −391G/A | −369A/G | −201A/G | −56C/T | +15A/G | +36A/G | AA31 (A/T) |
|---|---|---|---|---|---|---|---|---|---|---|---|---|
| 010102a | 010101b | 2 | 1 | 2 | 2 | 1 | 1 | 1 | 2 | 1 | 1 | 2 |
| 010102a | 010101c | 2 | 1 | 2 | 2 | 1 | 1 | 1 | 2 | 1 | 1 | 2 |
| 010102a | 010101d | 1 | 1 | 2 | 2 | 1 | 1 | 1 | 2 | 1 | 1 | 2 |
| 010102a | 010101e | 2 | 1 | 2 | 2 | 1 | 1 | 1 | 2 | 1 | 1 | 2 |
| 010102a | 010102a | 2 | 2 | 2 | 2 | 2 | 2 | 2 | 2 | 2 | 2 | 2 |
| 010102a | 010102b | 2 | 2 | 2 | 2 | 2 | 2 | 2 | 2 | 2 | 2 | 2 |
| 010102a | 010102c | 2 | 2 | 2 | 2 | 2 | 2 | 2 | 2 | 2 | 2 | 2 |
| 010102a | 010301a | 2 | 2 | 2 | 2 | 1 | 1 | 1 | 1 | 1 | 1 | 1 |
| 010102a | 010301b | 2 | 2 | 2 | 1 | 1 | 2 | 1 | 1 | 1 | 1 | 1 |
| 010102a | 010301c | 2 | 2 | 2 | 2 | 2 | 2 | 1 | 1 | 1 | 1 | 1 |
| 010102a | 010301d | 2 | 2 | 2 | 1 | 1 | 2 | 1 | 1 | 1 | 1 | 1 |
| 010102a | 010401a | 2 | 2 | 2 | 2 | 2 | 2 | 2 | 2 | 2 | 2 | 2 |
| 010102a | 010401b | 2 | 2 | 1 | 2 | 2 | 2 | 2 | 2 | 2 | 2 | 2 |
| 010102a | 0105N | 2 | 2 | 2 | 2 | 2 | 2 | 2 | 2 | 2 | 2 | 2 |
| 010102b | 010103 | 2 | 2 | 2 | 2 | 2 | 2 | 2 | 2 | 2 | 2 | 2 |
| 010102b | 010104 | 2 | 1 | 2 | 2 | 1 | 1 | 1 | 2 | 1 | 1 | 2 |
| 010102b | 010108 | 2 | 1 | 2 | 2 | 1 | 1 | 1 | 2 | 1 | 1 | 2 |
| 010102b | 010601 | 2 | 2 | 2 | 2 | 2 | 2 | 2 | 2 | 2 | 2 | 2 |
| 010102b | 010101a | 2 | 1 | 2 | 2 | 1 | 1 | 1 | 2 | 1 | 1 | 2 |
| 010102b | 010101b | 2 | 1 | 2 | 2 | 1 | 1 | 1 | 2 | 1 | 1 | 2 |
| 010102b | 010101c | 2 | 1 | 2 | 2 | 1 | 1 | 1 | 2 | 1 | 1 | 2 |
| 010102b | 010101d | 1 | 1 | 2 | 2 | 1 | 1 | 1 | 2 | 1 | 1 | 2 |
| 010102b | 010101e | 2 | 1 | 2 | 2 | 1 | 1 | 1 | 2 | 1 | 1 | 2 |
| 010102b | 010102b | 2 | 2 | 2 | 2 | 2 | 2 | 2 | 2 | 2 | 2 | 2 |
| 010102b | 010102c | 2 | 2 | 2 | 2 | 2 | 2 | 2 | 2 | 2 | 2 | 2 |
| 010102b | 010301a | 2 | 2 | 2 | 2 | 1 | 1 | 1 | 1 | 1 | 1 | 1 |
| 010102b | 010301b | 2 | 2 | 2 | 1 | 1 | 2 | 1 | 1 | 1 | 1 | 1 |
| 010102b | 010301c | 2 | 2 | 2 | 2 | 2 | 2 | 1 | 1 | 1 | 1 | 1 |
| 010102b | 010301d | 2 | 2 | 2 | 1 | 1 | 2 | 1 | 1 | 1 | 1 | 1 |
| 010102b | 010401a | 2 | 2 | 2 | 2 | 2 | 2 | 2 | 2 | 2 | 2 | 2 |
| 010102b | 010401b | 2 | 2 | 1 | 2 | 2 | 2 | 2 | 2 | 2 | 2 | 2 |

| | | AA35 | AA57 | AA69 | AA93 | AA100 | AA107 | AA110 | AA130 | | | |
|---|---|---|---|---|---|---|---|---|---|---|---|---|
| Allele1 | Allele2 | (A/T) | (A/G) | (C/T) | (T/C) | (C/T) | (A/T) | (C/A) | (C/D) | AA188 (C/T) | AA258 (C/T) | 14 bp in/del |
| 010102a | 010101b | 2 | 1 | 2 | 1 | 2 | 2 | 2 | 2 | 2 | 2 | 1 |
| 010102a | 010101c | 2 | 1 | 2 | 1 | 2 | 2 | 2 | 2 | 2 | 2 | 1 |
| 010102a | 010101d | 2 | 1 | 2 | 1 | 2 | 2 | 2 | 2 | 2 | 2 | 1 |
| 010102a | 010101e | 2 | 1 | 2 | 1 | 2 | 2 | 2 | 2 | 2 | 2 | 1 |
| 010102a | 010102a | 2 | 2 | 2 | 2 | 2 | 2 | 2 | 2 | 2 | 2 | 2 |
| 010102a | 010102b | 2 | 2 | 2 | 2 | 2 | 2 | 2 | 2 | 2 | 2 | 2 |
| 010102a | 010102c | 2 | 2 | 2 | 2 | 2 | 2 | 2 | 2 | 2 | 2 | 2 |
| 010102a | 010301a | 2 | 1 | 2 | 1 | 2 | 2 | 2 | 2 | 1 | 2 | 2 |
| 010102a | 010301b | 2 | 1 | 2 | 1 | 2 | 2 | 2 | 2 | 1 | 2 | 2 |
| 010102a | 010301c | 2 | 1 | 2 | 1 | 2 | 2 | 2 | 2 | 1 | 2 | 2 |
| 010102a | 010301d | 2 | 1 | 2 | 1 | 2 | 2 | 2 | 2 | 1 | 2 | 2 |
| 010102a | 010401a | 2 | 2 | 2 | 1 | 2 | 2 | 1 | 2 | 2 | 2 | 1 |
| 010102a | 010401b | 2 | 2 | 2 | 1 | 2 | 2 | 1 | 2 | 2 | 2 | 1 |
| 010102a | 0105N | 2 | 2 | 2 | 2 | 2 | 2 | 2 | 1 | 2 | 2 | 2 |
| 010102b | 010103 | 2 | 2 | 2 | 1 | 2 | 1 | 2 | 2 | 2 | 2 | 2 |
| 010102b | 010104 | 2 | 2 | 1 | 1 | 2 | 2 | 2 | 2 | 2 | 2 | 1 |
| 010102b | 010108 | 2 | 2 | 2 | 1 | 2 | 2 | 2 | 2 | 2 | 2 | 1 |
| 010102b | 010601 | 2 | 2 | 2 | 2 | 2 | 2 | 2 | 2 | 2 | 1 | 2 |
| 010102b | 010101a | 2 | 1 | 2 | 1 | 2 | 2 | 2 | 2 | 2 | 2 | 1 |
| 010102b | 010101b | 2 | 1 | 2 | 1 | 2 | 2 | 2 | 2 | 2 | 2 | 1 |
| 010102b | 010101c | 2 | 1 | 2 | 1 | 2 | 2 | 2 | 2 | 2 | 2 | 1 |
| 010102b | 010101d | 2 | 1 | 2 | 1 | 2 | 2 | 2 | 2 | 2 | 2 | 1 |
| 010102b | 010101e | 2 | 1 | 2 | 1 | 2 | 2 | 2 | 2 | 2 | 2 | 1 |
| 010102b | 010102b | 2 | 2 | 2 | 2 | 2 | 2 | 2 | 2 | 2 | 2 | 2 |
| 010102b | 010102c | 2 | 2 | 2 | 2 | 2 | 2 | 2 | 2 | 2 | 2 | 2 |
| 010102b | 010301a | 2 | 1 | 2 | 1 | 2 | 2 | 2 | 2 | 1 | 2 | 2 |
| 010102b | 010301b | 2 | 1 | 2 | 1 | 2 | 2 | 2 | 2 | 1 | 2 | 2 |
| 010102b | 010301c | 2 | 1 | 2 | 1 | 2 | 2 | 2 | 2 | 1 | 2 | 2 |
| 010102b | 010301d | 2 | 1 | 2 | 1 | 2 | 2 | 2 | 2 | 1 | 2 | 2 |
| 010102b | 010401a | 2 | 2 | 2 | 1 | 2 | 2 | 1 | 2 | 2 | 2 | 1 |
| 010102b | 010401b | 2 | 2 | 2 | 1 | 2 | 2 | 1 | 2 | 2 | 2 | 1 |

| Allele1 | Allele2 | −1306A/G | −1179G/A | −1155G/A | −1140T/A | −1138A/G | −1121C/T | −990G/A | −0964A/G | −922C/A | −810C/T |
|---|---|---|---|---|---|---|---|---|---|---|---|
| 010102b | 0105N | 2 | 2 | 2 | 2 | 2 | 2 | 2 | 2 | 2 | 1 |
| 010102c | 010103 | 2 | 2 | 2 | 2 | 2 | 2 | 2 | 2 | 1 | 2 |
| 010102c | 010104 | 1 | 1 | 2 | 1 | 2 | 2 | 1 | 1 | 1 | 2 |
| 010102c | 010108 | 1 | 1 | 2 | 1 | 2 | 2 | 1 | 1 | 1 | 2 |
| 010102c | 010601 | 2 | 2 | 2 | 2 | 2 | 2 | 2 | 2 | 1 | 2 |
| 010102c | 010101a | 1 | 1 | 2 | 1 | 2 | 2 | 2 | 1 | 1 | 2 |
| 010102c | 010101b | 1 | 1 | 2 | 1 | 2 | 2 | 2 | 1 | 1 | 2 |
| 010102c | 010101c | 1 | 1 | 2 | 1 | 2 | 1 | 2 | 1 | 1 | 2 |
| 010102c | 010101d | 1 | 1 | 2 | 1 | 2 | 2 | 2 | 1 | 1 | 2 |
| 010102c | 010101e | 1 | 1 | 2 | 1 | 2 | 2 | 1 | 1 | 1 | 2 |
| 010102c | 010102c | 2 | 2 | 2 | 2 | 2 | 2 | 2 | 2 | 0 | 2 |
| 010102c | 010301a | 1 | 2 | 2 | 1 | 1 | 1 | 2 | 1 | 1 | 2 |
| 010102c | 010301b | 1 | 2 | 2 | 1 | 1 | 1 | 2 | 1 | 1 | 2 |
| 010102c | 010301c | 1 | 2 | 2 | 1 | 1 | 1 | 2 | 1 | 1 | 2 |
| 010102c | 010301d | 1 | 2 | 2 | 1 | 1 | 1 | 2 | 1 | 1 | 2 |
| 010102c | 010401a | 2 | 2 | 1 | 1 | 2 | 2 | 2 | 2 | 1 | 2 |
| 010102c | 010401b | 2 | 2 | 1 | 1 | 2 | 2 | 2 | 2 | 1 | 2 |
| 010102c | 0105N | 2 | 2 | 2 | 2 | 2 | 2 | 2 | 2 | 1 | 2 |
| 010301a | 010104 | 0 | 1 | 2 | 0 | 1 | 1 | 1 | 0 | 2 | 2 |
| 010301a | 010108 | 0 | 1 | 2 | 0 | 1 | 1 | 1 | 0 | 2 | 2 |
| 010301a | 010101a | 0 | 1 | 2 | 0 | 1 | 1 | 2 | 0 | 2 | 2 |
| 010301a | 010101b | 0 | 1 | 2 | 0 | 1 | 1 | 2 | 0 | 2 | 2 |
| 010301a | 010101c | 0 | 1 | 2 | 0 | 1 | 0 | 2 | 0 | 2 | 2 |
| 010301a | 010101d | 0 | 1 | 2 | 0 | 1 | 1 | 2 | 0 | 2 | 2 |
| 010301a | 010101e | 0 | 1 | 2 | 0 | 1 | 1 | 1 | 0 | 2 | 2 |
| 010301a | 010301a | 0 | 2 | 2 | 0 | 0 | 0 | 2 | 0 | 2 | 2 |
| 010301a | 010301b | 0 | 2 | 2 | 0 | 0 | 0 | 2 | 0 | 2 | 2 |
| 010301a | 010301c | 0 | 2 | 2 | 0 | 0 | 0 | 2 | 0 | 2 | 2 |
| 010301a | 010301d | 0 | 2 | 2 | 0 | 0 | 0 | 2 | 0 | 2 | 2 |
| 010301a | 010401a | 1 | 2 | 1 | 0 | 1 | 1 | 2 | 1 | 2 | 2 |
| 010301a | 010401b | 1 | 2 | 1 | 0 | 1 | 1 | 2 | 1 | 2 | 2 |

| Allele1 | Allele2 | −762T/C | −725C | −725G | −725T | −716G/T | −689G/A | −666T/G | −649A/G | −633A/G | −509C/G | −486C/A |
|---|---|---|---|---|---|---|---|---|---|---|---|---|
| 010102b | 0105N | 2 | 2 | 0 | 0 | 2 | 2 | 2 | 2 | 2 | 2 | 2 |
| 010102c | 010103 | 2 | 2 | 0 | 0 | 2 | 2 | 2 | 2 | 2 | 2 | 2 |
| 010102c | 010104 | 1 | 2 | 0 | 0 | 1 | 1 | 1 | 2 | 1 | 2 | 1 |
| 010102c | 010108 | 1 | 2 | 0 | 0 | 1 | 1 | 1 | 2 | 1 | 2 | 1 |
| 010102c | 010601 | 2 | 2 | 0 | 0 | 2 | 2 | 2 | 2 | 2 | 2 | 2 |
| 010102c | 010101a | 1 | 2 | 0 | 0 | 1 | 1 | 1 | 2 | 1 | 2 | 1 |
| 010102c | 010101b | 1 | 1 | 1 | 0 | 1 | 1 | 1 | 2 | 1 | 2 | 1 |
| 010102c | 010101c | 1 | 1 | 1 | 0 | 1 | 1 | 1 | 2 | 1 | 2 | 1 |
| 010102c | 010101d | 1 | 2 | 0 | 0 | 1 | 1 | 1 | 2 | 1 | 2 | 1 |
| 010102c | 010101e | 1 | 2 | 0 | 0 | 1 | 1 | 1 | 2 | 1 | 2 | 1 |
| 010102c | 010102c | 2 | 2 | 0 | 0 | 2 | 2 | 2 | 2 | 2 | 2 | 2 |
| 010102c | 010301a | 1 | 1 | 0 | 1 | 1 | 1 | 1 | 2 | 1 | 1 | 1 |
| 010102c | 010301b | 1 | 1 | 0 | 1 | 1 | 1 | 1 | 1 | 1 | 1 | 1 |
| 010102c | 010301c | 1 | 1 | 0 | 1 | 1 | 1 | 1 | 1 | 1 | 1 | 1 |
| 010102c | 010301d | 1 | 1 | 0 | 1 | 1 | 1 | 1 | 2 | 1 | 2 | 1 |
| 010102c | 010401a | 2 | 2 | 0 | 0 | 2 | 2 | 2 | 2 | 2 | 2 | 2 |
| 010102c | 010401b | 2 | 2 | 0 | 0 | 2 | 2 | 2 | 2 | 2 | 2 | 2 |
| 010102c | 0105N | 2 | 2 | 0 | 0 | 2 | 2 | 2 | 2 | 2 | 2 | 2 |
| 010301a | 010104 | 0 | 1 | 0 | 1 | 0 | 0 | 0 | 2 | 0 | 1 | 0 |
| 010301a | 010108 | 0 | 1 | 0 | 1 | 0 | 0 | 0 | 2 | 0 | 1 | 0 |
| 010301a | 010101a | 0 | 1 | 0 | 1 | 0 | 0 | 0 | 2 | 0 | 1 | 0 |
| 010301a | 010101b | 0 | 0 | 1 | 1 | 0 | 0 | 0 | 2 | 0 | 1 | 0 |
| 010301a | 010101c | 0 | 0 | 1 | 1 | 0 | 0 | 0 | 2 | 0 | 1 | 0 |
| 010301a | 010101d | 0 | 1 | 0 | 1 | 0 | 0 | 0 | 2 | 0 | 1 | 0 |
| 010301a | 010101e | 0 | 1 | 0 | 1 | 0 | 0 | 0 | 2 | 0 | 1 | 0 |
| 010301a | 010301a | 0 | 0 | 0 | 2 | 0 | 0 | 0 | 2 | 0 | 0 | 0 |
| 010301a | 010301b | 0 | 0 | 0 | 2 | 0 | 0 | 0 | 1 | 0 | 0 | 0 |
| 010301a | 010301c | 0 | 0 | 0 | 2 | 0 | 0 | 0 | 1 | 0 | 0 | 0 |
| 010301a | 010301d | 0 | 0 | 0 | 2 | 0 | 0 | 0 | 2 | 0 | 1 | 0 |
| 010301a | 010401a | 1 | 1 | 0 | 1 | 1 | 1 | 1 | 2 | 1 | 1 | 1 |
| 010301a | 010401b | 1 | 1 | 0 | 1 | 1 | 1 | 1 | 2 | 1 | 1 | 1 |

| Allele1 | Allele2 | −482A/G | −477G/C | −443G/A | −400G/A | −391G/A | −369A/G | −201A/G | −56C/T | +15A/G | +36A/G | AA31 (A/T) |
|---|---|---|---|---|---|---|---|---|---|---|---|---|
| 010102b | 0105N | 2 | 2 | 2 | 2 | 2 | 2 | 2 | 2 | 2 | 2 | 2 |
| 010102c | 010103 | 2 | 2 | 2 | 2 | 2 | 2 | 2 | 2 | 2 | 2 | 2 |
| 010102c | 010104 | 2 | 1 | 2 | 2 | 1 | 1 | 1 | 2 | 1 | 1 | 2 |
| 010102c | 010108 | 2 | 1 | 2 | 2 | 1 | 1 | 1 | 2 | 1 | 1 | 2 |
| 010102c | 010601 | 2 | 2 | 2 | 2 | 2 | 2 | 2 | 2 | 2 | 2 | 2 |
| 010102c | 010101a | 2 | 1 | 2 | 2 | 1 | 1 | 1 | 2 | 1 | 1 | 2 |
| 010102c | 010101b | 2 | 1 | 2 | 2 | 1 | 1 | 1 | 2 | 1 | 1 | 2 |
| 010102c | 010101c | 2 | 1 | 2 | 2 | 1 | 1 | 1 | 2 | 1 | 1 | 2 |
| 010102c | 010101d | 1 | 1 | 2 | 2 | 1 | 1 | 1 | 2 | 1 | 1 | 2 |
| 010102c | 010101e | 2 | 1 | 2 | 2 | 1 | 1 | 1 | 2 | 1 | 1 | 2 |
| 010102c | 010102c | 2 | 2 | 2 | 2 | 2 | 2 | 2 | 2 | 2 | 2 | 2 |
| 010102c | 010301a | 2 | 2 | 2 | 2 | 1 | 1 | 1 | 1 | 1 | 1 | 1 |
| 010102c | 010301b | 2 | 2 | 2 | 1 | 1 | 2 | 1 | 1 | 1 | 1 | 1 |
| 010102c | 010301c | 2 | 2 | 2 | 2 | 2 | 2 | 1 | 1 | 1 | 1 | 1 |
| 010102c | 010301d | 2 | 2 | 2 | 1 | 1 | 2 | 1 | 1 | 1 | 1 | 1 |
| 010102c | 010401a | 2 | 2 | 2 | 2 | 2 | 2 | 2 | 2 | 2 | 2 | 2 |
| 010102c | 010401b | 2 | 2 | 1 | 2 | 2 | 2 | 2 | 2 | 2 | 2 | 2 |
| 010102c | 0105N | 2 | 2 | 2 | 2 | 2 | 2 | 2 | 2 | 2 | 2 | 2 |
| 010301a | 010104 | 2 | 1 | 2 | 2 | 0 | 0 | 0 | 1 | 0 | 0 | 1 |
| 010301a | 010108 | 2 | 1 | 2 | 2 | 0 | 0 | 0 | 1 | 0 | 0 | 1 |
| 010301a | 010101a | 2 | 1 | 2 | 2 | 0 | 0 | 0 | 1 | 0 | 0 | 1 |
| 010301a | 010101b | 2 | 1 | 2 | 2 | 0 | 0 | 0 | 1 | 0 | 0 | 1 |
| 010301a | 010101c | 2 | 1 | 2 | 2 | 0 | 0 | 0 | 1 | 0 | 0 | 1 |
| 010301a | 010101d | 1 | 1 | 2 | 2 | 0 | 0 | 0 | 1 | 0 | 0 | 1 |
| 010301a | 010101e | 2 | 1 | 2 | 2 | 0 | 0 | 0 | 1 | 0 | 0 | 1 |
| 010301a | 010301a | 2 | 2 | 2 | 2 | 0 | 0 | 0 | 0 | 0 | 0 | 0 |
| 010301a | 010301b | 2 | 2 | 2 | 1 | 0 | 1 | 0 | 0 | 0 | 0 | 0 |
| 010301a | 010301c | 2 | 2 | 2 | 2 | 1 | 1 | 0 | 0 | 0 | 0 | 0 |
| 010301a | 010301d | 2 | 2 | 2 | 1 | 0 | 1 | 0 | 0 | 0 | 0 | 0 |
| 010301a | 010401a | 2 | 2 | 2 | 2 | 1 | 1 | 1 | 1 | 1 | 1 | 1 |
| 010301a | 010401b | 2 | 2 | 1 | 2 | 1 | 1 | 1 | 1 | 1 | 1 | 1 |

| | | AA35 | AA57 | AA69 | AA93 | AA100 | AA107 | AA110 | AA130 | | | |
|---|---|---|---|---|---|---|---|---|---|---|---|---|
| Allele1 | Allele2 | (A/T) | (A/G) | (C/T) | (T/C) | (C/T) | (A/T) | (C/A) | (C/D) | AA188 (C/T) | AA258 (C/T) | 14 bp in/del |
| 010102b | 0105N | 2 | 2 | 2 | 2 | 2 | 2 | 2 | 1 | 2 | 2 | 2 |
| 010102c | 010103 | 2 | 2 | 2 | 1 | 2 | 1 | 2 | 2 | 2 | 2 | 2 |
| 010102c | 010104 | 2 | 2 | 1 | 1 | 2 | 2 | 2 | 2 | 2 | 2 | 1 |
| 010102c | 010108 | 2 | 2 | 2 | 1 | 2 | 2 | 2 | 2 | 2 | 2 | 1 |
| 010102c | 010601 | 2 | 2 | 2 | 2 | 2 | 2 | 2 | 2 | 2 | 1 | 2 |
| 010102c | 010101a | 2 | 1 | 2 | 1 | 2 | 2 | 2 | 2 | 2 | 2 | 1 |
| 010102c | 010101b | 2 | 1 | 2 | 1 | 2 | 2 | 2 | 2 | 2 | 2 | 1 |
| 010102c | 010101c | 2 | 1 | 2 | 1 | 2 | 2 | 2 | 2 | 2 | 2 | 1 |
| 010102c | 010101d | 2 | 1 | 2 | 1 | 2 | 2 | 2 | 2 | 2 | 2 | 1 |
| 010102c | 010101e | 2 | 1 | 2 | 1 | 2 | 2 | 2 | 2 | 2 | 2 | 1 |
| 010102c | 010102c | 2 | 2 | 2 | 2 | 2 | 2 | 2 | 2 | 2 | 2 | 2 |
| 010102c | 010301a | 2 | 1 | 2 | 1 | 2 | 2 | 2 | 2 | 1 | 2 | 2 |
| 010102c | 010301b | 2 | 1 | 2 | 1 | 2 | 2 | 2 | 2 | 1 | 2 | 2 |
| 010102c | 010301c | 2 | 1 | 2 | 1 | 2 | 2 | 2 | 2 | 1 | 2 | 2 |
| 010102c | 010301d | 2 | 1 | 2 | 1 | 2 | 2 | 2 | 2 | 1 | 2 | 2 |
| 010102c | 010401a | 2 | 2 | 2 | 1 | 2 | 2 | 1 | 2 | 2 | 2 | 1 |
| 010102c | 010401b | 2 | 2 | 2 | 1 | 2 | 2 | 1 | 2 | 2 | 2 | 1 |
| 010102c | 0105N | 2 | 2 | 2 | 2 | 2 | 2 | 2 | 1 | 2 | 2 | 2 |
| 010301a | 010104 | 2 | 1 | 1 | 0 | 2 | 2 | 2 | 2 | 1 | 2 | 1 |
| 010301a | 010108 | 2 | 1 | 2 | 0 | 2 | 2 | 2 | 2 | 1 | 2 | 1 |
| 010301a | 010101a | 2 | 0 | 2 | 0 | 2 | 2 | 2 | 2 | 1 | 2 | 1 |
| 010301a | 010101b | 2 | 0 | 2 | 0 | 2 | 2 | 2 | 2 | 1 | 2 | 1 |
| 010301a | 010101c | 2 | 0 | 2 | 0 | 2 | 2 | 2 | 2 | 1 | 2 | 1 |
| 010301a | 010101d | 2 | 0 | 2 | 0 | 2 | 2 | 2 | 2 | 1 | 2 | 1 |
| 010301a | 010101e | 2 | 0 | 2 | 0 | 2 | 2 | 2 | 2 | 1 | 2 | 1 |
| 010301a | 010301a | 2 | 0 | 2 | 0 | 2 | 2 | 2 | 2 | 0 | 2 | 2 |
| 010301a | 010301b | 2 | 0 | 2 | 0 | 2 | 2 | 2 | 2 | 0 | 2 | 2 |
| 010301a | 010301c | 2 | 0 | 2 | 0 | 2 | 2 | 2 | 2 | 0 | 2 | 2 |
| 010301a | 010301d | 2 | 0 | 2 | 0 | 2 | 2 | 2 | 2 | 0 | 2 | 2 |
| 010301a | 010401a | 2 | 1 | 2 | 0 | 2 | 2 | 1 | 2 | 1 | 2 | 1 |
| 010301a | 010401b | 2 | 1 | 2 | 0 | 2 | 2 | 1 | 2 | 1 | 2 | 1 |

| Allele1 | Allele2 | −1306A/G | −1179G/A | −1155G/A | −1140T/A | −1138A/G | −1121C/T | −990G/A | −0964A/G | −922C/A | −810C/T |
|---|---|---|---|---|---|---|---|---|---|---|---|
| 010301b | 010104 | 0 | 1 | 2 | 0 | 1 | 1 | 1 | 0 | 2 | 2 |
| 010301b | 010108 | 0 | 1 | 2 | 0 | 1 | 1 | 1 | 0 | 2 | 2 |
| 010301b | 010101a | 0 | 1 | 2 | 0 | 1 | 1 | 2 | 0 | 2 | 2 |
| 010301b | 010101b | 0 | 1 | 2 | 0 | 1 | 1 | 2 | 0 | 2 | 2 |
| 010301b | 010101c | 0 | 1 | 2 | 0 | 1 | 0 | 2 | 0 | 2 | 2 |
| 010301b | 010101d | 0 | 1 | 2 | 0 | 1 | 1 | 2 | 0 | 2 | 2 |
| 010301b | 010101e | 0 | 1 | 2 | 0 | 1 | 1 | 1 | 0 | 2 | 2 |
| 010301b | 010301b | 0 | 2 | 2 | 0 | 0 | 0 | 2 | 0 | 2 | 2 |
| 010301b | 010301c | 0 | 2 | 2 | 0 | 0 | 0 | 2 | 0 | 2 | 2 |
| 010301b | 010301d | 0 | 2 | 2 | 0 | 0 | 0 | 2 | 0 | 2 | 2 |
| 010301b | 010401a | 1 | 2 | 1 | 0 | 1 | 1 | 2 | 1 | 2 | 2 |
| 010301b | 010401b | 1 | 2 | 1 | 0 | 1 | 1 | 2 | 1 | 2 | 2 |
| 010301c | 010104 | 0 | 1 | 2 | 0 | 1 | 1 | 1 | 0 | 2 | 2 |
| 010301c | 010108 | 0 | 1 | 2 | 0 | 1 | 1 | 1 | 0 | 2 | 2 |
| 010301c | 010101a | 0 | 1 | 2 | 0 | 1 | 1 | 2 | 0 | 2 | 2 |
| 010301c | 010101b | 0 | 1 | 2 | 0 | 1 | 1 | 2 | 0 | 2 | 2 |
| 010301c | 010101c | 0 | 1 | 2 | 0 | 1 | 0 | 2 | 0 | 2 | 2 |
| 010301c | 010101d | 0 | 1 | 2 | 0 | 1 | 1 | 2 | 0 | 2 | 2 |
| 010301c | 010101e | 0 | 1 | 2 | 0 | 1 | 1 | 1 | 0 | 2 | 2 |
| 010301c | 010301c | 0 | 2 | 2 | 0 | 0 | 0 | 2 | 0 | 2 | 2 |
| 010301c | 010301d | 0 | 2 | 2 | 0 | 0 | 0 | 2 | 0 | 2 | 2 |
| 010301c | 010401a | 1 | 2 | 1 | 0 | 1 | 1 | 2 | 1 | 2 | 2 |
| 010301c | 010401b | 1 | 2 | 1 | 0 | 1 | 1 | 2 | 1 | 2 | 2 |
| 010301d | 010104 | 0 | 1 | 2 | 0 | 1 | 1 | 1 | 0 | 2 | 2 |
| 010301d | 010108 | 0 | 1 | 2 | 0 | 1 | 1 | 1 | 0 | 2 | 2 |
| 010301d | 010101a | 0 | 1 | 2 | 0 | 1 | 1 | 2 | 0 | 2 | 2 |
| 010301d | 010101b | 0 | 1 | 2 | 0 | 1 | 1 | 2 | 0 | 2 | 2 |
| 010301d | 010101c | 0 | 1 | 2 | 0 | 1 | 0 | 2 | 0 | 2 | 2 |
| 010301d | 010101d | 0 | 1 | 2 | 0 | 1 | 1 | 2 | 0 | 2 | 2 |
| 010301d | 010101e | 0 | 1 | 2 | 0 | 1 | 1 | 1 | 0 | 2 | 2 |
| 010301d | 010301d | 0 | 2 | 2 | 0 | 0 | 0 | 2 | 0 | 2 | 2 |

| Allele1 | Allele2 | −762T/C | −725C | −725G | −725T | −716G/T | −689G/A | −666T/G | −649A/G | −633A/G | −509C/G | −486C/A |
|---|---|---|---|---|---|---|---|---|---|---|---|---|
| 010301b | 010104 | 0 | 1 | 0 | 1 | 0 | 0 | 0 | 1 | 0 | 1 | 0 |
| 010301b | 010108 | 0 | 1 | 0 | 1 | 0 | 0 | 0 | 1 | 0 | 1 | 0 |
| 010301b | 010101a | 0 | 1 | 0 | 1 | 0 | 0 | 0 | 1 | 0 | 1 | 0 |
| 010301b | 010101b | 0 | 0 | 1 | 1 | 0 | 0 | 0 | 1 | 0 | 1 | 0 |
| 010301b | 010101c | 0 | 0 | 1 | 1 | 0 | 0 | 0 | 1 | 0 | 1 | 0 |
| 010301b | 010101d | 0 | 1 | 0 | 1 | 0 | 0 | 0 | 1 | 0 | 1 | 0 |
| 010301b | 010101e | 0 | 1 | 0 | 1 | 0 | 0 | 0 | 1 | 0 | 1 | 0 |
| 010301b | 010301b | 0 | 0 | 0 | 2 | 0 | 0 | 0 | 0 | 0 | 0 | 0 |
| 010301b | 010301c | 0 | 0 | 0 | 2 | 0 | 0 | 0 | 0 | 0 | 0 | 0 |
| 010301b | 010301d | 0 | 0 | 0 | 2 | 0 | 0 | 0 | 1 | 0 | 1 | 0 |
| 010301b | 010401a | 1 | 1 | 0 | 1 | 1 | 1 | 1 | 1 | 1 | 1 | 1 |
| 010301b | 010401b | 1 | 1 | 0 | 1 | 1 | 1 | 1 | 1 | 1 | 1 | 1 |
| 010301c | 010104 | 0 | 1 | 0 | 1 | 0 | 0 | 0 | 1 | 0 | 1 | 0 |
| 010301c | 010108 | 0 | 1 | 0 | 1 | 0 | 0 | 0 | 1 | 0 | 1 | 0 |
| 010301c | 010101a | 0 | 1 | 0 | 1 | 0 | 0 | 0 | 1 | 0 | 1 | 0 |
| 010301c | 010101b | 0 | 0 | 1 | 1 | 0 | 0 | 0 | 1 | 0 | 1 | 0 |
| 010301c | 010101c | 0 | 0 | 1 | 1 | 0 | 0 | 0 | 1 | 0 | 1 | 0 |
| 010301c | 010101d | 0 | 1 | 0 | 1 | 0 | 0 | 0 | 1 | 0 | 1 | 0 |
| 010301c | 010101e | 0 | 1 | 0 | 1 | 0 | 0 | 0 | 1 | 0 | 1 | 0 |
| 010301c | 010301c | 0 | 0 | 0 | 2 | 0 | 0 | 0 | 0 | 0 | 0 | 0 |
| 010301c | 010301d | 0 | 0 | 0 | 2 | 0 | 0 | 0 | 1 | 0 | 1 | 0 |
| 010301c | 010401a | 1 | 1 | 0 | 1 | 1 | 1 | 1 | 1 | 1 | 1 | 1 |
| 010301c | 010401b | 1 | 1 | 0 | 1 | 1 | 1 | 1 | 1 | 1 | 1 | 1 |
| 010301d | 010104 | 0 | 1 | 0 | 1 | 0 | 0 | 0 | 2 | 0 | 2 | 0 |
| 010301d | 010108 | 0 | 1 | 0 | 1 | 0 | 0 | 0 | 2 | 0 | 2 | 0 |
| 010301d | 010101a | 0 | 1 | 0 | 1 | 0 | 0 | 0 | 2 | 0 | 2 | 0 |
| 010301d | 010101b | 0 | 0 | 1 | 1 | 0 | 0 | 0 | 2 | 0 | 2 | 0 |
| 010301d | 010101c | 0 | 0 | 1 | 1 | 0 | 0 | 0 | 2 | 0 | 2 | 0 |
| 010301d | 010101d | 0 | 1 | 0 | 1 | 0 | 0 | 0 | 2 | 0 | 2 | 0 |
| 010301d | 010101e | 0 | 1 | 0 | 1 | 0 | 0 | 0 | 2 | 0 | 2 | 0 |
| 010301d | 010301d | 0 | 0 | 0 | 2 | 0 | 0 | 0 | 2 | 0 | 2 | 0 |

| Allele1 | Allele2 | −482A/G | −477G/C | −443G/A | −400G/A | −391G/A | −369A/G | −201A/G | −56C/T | +15A/G | +36A/G | AA31 (A/T) |
|---|---|---|---|---|---|---|---|---|---|---|---|---|
| 010301b | 010104 | 2 | 1 | 2 | 1 | 0 | 1 | 0 | 1 | 0 | 0 | 1 |
| 010301b | 010108 | 2 | 1 | 2 | 1 | 0 | 1 | 0 | 1 | 0 | 0 | 1 |
| 010301b | 010101a | 2 | 1 | 2 | 1 | 0 | 1 | 0 | 1 | 0 | 0 | 1 |
| 010301b | 010101b | 2 | 1 | 2 | 1 | 0 | 1 | 0 | 1 | 0 | 0 | 1 |
| 010301b | 010101c | 2 | 1 | 2 | 1 | 0 | 1 | 0 | 1 | 0 | 0 | 1 |
| 010301b | 010101d | 1 | 1 | 2 | 1 | 0 | 1 | 0 | 1 | 0 | 0 | 1 |
| 010301b | 010101e | 2 | 1 | 2 | 1 | 0 | 1 | 0 | 1 | 0 | 0 | 1 |
| 010301b | 010301b | 2 | 2 | 2 | 0 | 0 | 2 | 0 | 0 | 0 | 0 | 0 |
| 010301b | 010301c | 2 | 2 | 2 | 1 | 1 | 2 | 0 | 0 | 0 | 0 | 0 |
| 010301b | 010301d | 2 | 2 | 2 | 0 | 0 | 2 | 0 | 0 | 0 | 0 | 0 |
| 010301b | 010401a | 2 | 2 | 2 | 1 | 1 | 2 | 1 | 1 | 1 | 1 | 1 |
| 010301b | 010401b | 2 | 2 | 1 | 1 | 1 | 2 | 1 | 1 | 1 | 1 | 1 |
| 010301c | 010104 | 2 | 1 | 2 | 2 | 1 | 1 | 0 | 1 | 0 | 0 | 1 |
| 010301c | 010108 | 2 | 1 | 2 | 2 | 1 | 1 | 0 | 1 | 0 | 0 | 1 |
| 010301c | 010101a | 2 | 1 | 2 | 2 | 1 | 1 | 0 | 1 | 0 | 0 | 1 |
| 010301c | 010101b | 2 | 1 | 2 | 2 | 1 | 1 | 0 | 1 | 0 | 0 | 1 |
| 010301c | 010101c | 2 | 1 | 2 | 2 | 1 | 1 | 0 | 1 | 0 | 0 | 1 |
| 010301c | 010101d | 1 | 1 | 2 | 2 | 1 | 1 | 0 | 1 | 0 | 0 | 1 |
| 010301c | 010101e | 2 | 1 | 2 | 2 | 1 | 1 | 0 | 1 | 0 | 0 | 1 |
| 010301c | 010301c | 2 | 2 | 2 | 2 | 2 | 2 | 0 | 0 | 0 | 0 | 0 |
| 010301c | 010301d | 2 | 2 | 2 | 1 | 1 | 2 | 0 | 0 | 0 | 0 | 0 |
| 010301c | 010401a | 2 | 2 | 2 | 2 | 2 | 2 | 1 | 1 | 1 | 1 | 1 |
| 010301c | 010401b | 2 | 2 | 1 | 2 | 2 | 2 | 1 | 1 | 1 | 1 | 1 |
| 010301d | 010104 | 2 | 1 | 2 | 1 | 0 | 1 | 0 | 1 | 0 | 0 | 1 |
| 010301d | 010108 | 2 | 1 | 2 | 1 | 0 | 1 | 0 | 1 | 0 | 0 | 1 |
| 010301d | 010101a | 2 | 1 | 2 | 1 | 0 | 1 | 0 | 1 | 0 | 0 | 1 |
| 010301d | 010101b | 2 | 1 | 2 | 1 | 0 | 1 | 0 | 1 | 0 | 0 | 1 |
| 010301d | 010101c | 2 | 1 | 2 | 1 | 0 | 1 | 0 | 1 | 0 | 0 | 1 |
| 010301d | 010101d | 1 | 1 | 2 | 1 | 0 | 1 | 0 | 1 | 0 | 0 | 1 |
| 010301d | 010101e | 2 | 1 | 2 | 1 | 0 | 1 | 0 | 1 | 0 | 0 | 1 |
| 010301d | 010301d | 2 | 2 | 2 | 0 | 0 | 2 | 0 | 0 | 0 | 0 | 0 |

| | | AA35 | AA57 | AA69 | AA93 | AA100 | AA107 | AA110 | AA130 | | | |
|---|---|---|---|---|---|---|---|---|---|---|---|---|
| Allele1 | Allele2 | (A/T) | (A/G) | (C/T) | (T/C) | (C/T) | (A/T) | (C/A) | (C/D) | AA188 (C/T) | AA258 (C/T) | 14 bp in/del |
| 010301b | 010104 | 2 | 1 | 1 | 0 | 2 | 2 | 2 | 2 | 1 | 2 | 1 |
| 010301b | 010108 | 2 | 1 | 2 | 0 | 2 | 2 | 2 | 2 | 1 | 2 | 1 |
| 010301b | 010101a | 2 | 0 | 2 | 0 | 2 | 2 | 2 | 2 | 1 | 2 | 1 |
| 010301b | 010101b | 2 | 0 | 2 | 0 | 2 | 2 | 2 | 2 | 1 | 2 | 1 |
| 010301b | 010101c | 2 | 0 | 2 | 0 | 2 | 2 | 2 | 2 | 1 | 2 | 1 |
| 010301b | 010101d | 2 | 0 | 2 | 0 | 2 | 2 | 2 | 2 | 1 | 2 | 1 |
| 010301b | 010101e | 2 | 0 | 2 | 0 | 2 | 2 | 2 | 2 | 1 | 2 | 1 |
| 010301b | 010301b | 2 | 0 | 2 | 0 | 2 | 2 | 2 | 2 | 0 | 2 | 2 |
| 010301b | 010301c | 2 | 0 | 2 | 0 | 2 | 2 | 2 | 2 | 0 | 2 | 2 |
| 010301b | 010301d | 2 | 0 | 2 | 0 | 2 | 2 | 2 | 2 | 0 | 2 | 2 |
| 010301b | 010401a | 2 | 1 | 2 | 0 | 2 | 2 | 1 | 2 | 1 | 2 | 1 |
| 010301b | 010401b | 2 | 1 | 2 | 0 | 2 | 2 | 1 | 2 | 1 | 2 | 1 |
| 010301c | 010104 | 2 | 1 | 1 | 0 | 2 | 2 | 2 | 2 | 1 | 2 | 1 |
| 010301c | 010108 | 2 | 1 | 2 | 0 | 2 | 2 | 2 | 2 | 1 | 2 | 1 |
| 010301c | 010101a | 2 | 0 | 2 | 0 | 2 | 2 | 2 | 2 | 1 | 2 | 1 |
| 010301c | 010101b | 2 | 0 | 2 | 0 | 2 | 2 | 2 | 2 | 1 | 2 | 1 |
| 010301c | 010101c | 2 | 0 | 2 | 0 | 2 | 2 | 2 | 2 | 1 | 2 | 1 |
| 010301c | 010101d | 2 | 0 | 2 | 0 | 2 | 2 | 2 | 2 | 1 | 2 | 1 |
| 010301c | 010101e | 2 | 0 | 2 | 0 | 2 | 2 | 2 | 2 | 1 | 2 | 1 |
| 010301c | 010301c | 2 | 0 | 2 | 0 | 2 | 2 | 2 | 2 | 0 | 2 | 2 |
| 010301c | 010301d | 2 | 0 | 2 | 0 | 2 | 2 | 2 | 2 | 0 | 2 | 2 |
| 010301c | 010401a | 2 | 1 | 2 | 0 | 2 | 2 | 1 | 2 | 1 | 2 | 1 |
| 010301c | 010401b | 2 | 1 | 2 | 0 | 2 | 2 | 1 | 2 | 1 | 2 | 1 |
| 010301d | 010104 | 2 | 1 | 1 | 0 | 2 | 2 | 2 | 2 | 1 | 2 | 1 |
| 010301d | 010108 | 2 | 1 | 2 | 0 | 2 | 2 | 2 | 2 | 1 | 2 | 1 |
| 010301d | 010101a | 2 | 0 | 2 | 0 | 2 | 2 | 2 | 2 | 1 | 2 | 1 |
| 010301d | 010101b | 2 | 0 | 2 | 0 | 2 | 2 | 2 | 2 | 1 | 2 | 1 |
| 010301d | 010101c | 2 | 0 | 2 | 0 | 2 | 2 | 2 | 2 | 1 | 2 | 1 |
| 010301d | 010101d | 2 | 0 | 2 | 0 | 2 | 2 | 2 | 2 | 1 | 2 | 1 |
| 010301d | 010101e | 2 | 0 | 2 | 0 | 2 | 2 | 2 | 2 | 1 | 2 | 1 |
| 010301d | 010301d | 2 | 0 | 2 | 0 | 2 | 2 | 2 | 2 | 0 | 2 | 2 |

| Allele1 | Allele2 | −1306A/G | −1179G/A | −1155G/A | −1140T/A | −1138A/G | −1121C/T | −990G/A | −0964A/G | −922C/A | −810C/T |
|---|---|---|---|---|---|---|---|---|---|---|---|
| 010301d | 010401a | 1 | 2 | 1 | 0 | 1 | 1 | 2 | 1 | 2 | 2 |
| 010301d | 010401b | 1 | 2 | 1 | 0 | 1 | 1 | 2 | 1 | 2 | 2 |
| 010401a | 010104 | 1 | 1 | 1 | 0 | 2 | 2 | 1 | 1 | 2 | 2 |
| 010401a | 010108 | 1 | 1 | 1 | 0 | 2 | 2 | 1 | 1 | 2 | 2 |
| 010401a | 010101a | 1 | 1 | 1 | 0 | 2 | 2 | 2 | 1 | 2 | 2 |
| 010401a | 010101b | 1 | 1 | 1 | 0 | 2 | 2 | 2 | 1 | 2 | 2 |
| 010401a | 010101c | 1 | 1 | 1 | 0 | 2 | 1 | 2 | 1 | 2 | 2 |
| 010401a | 010101d | 1 | 1 | 1 | 0 | 2 | 2 | 2 | 1 | 2 | 2 |
| 010401a | 010101e | 1 | 1 | 1 | 0 | 2 | 2 | 1 | 1 | 2 | 2 |
| 010401a | 010401a | 2 | 2 | 0 | 0 | 2 | 2 | 2 | 2 | 2 | 2 |
| 010401a | 010401b | 2 | 2 | 0 | 0 | 2 | 2 | 2 | 2 | 2 | 2 |
| 010401b | 010104 | 1 | 1 | 1 | 0 | 2 | 2 | 1 | 1 | 2 | 2 |
| 010401b | 010108 | 1 | 1 | 1 | 0 | 2 | 2 | 1 | 1 | 2 | 2 |
| 010401b | 010101a | 1 | 1 | 1 | 0 | 2 | 2 | 2 | 1 | 2 | 2 |
| 010401b | 010101b | 1 | 1 | 1 | 0 | 2 | 2 | 2 | 1 | 2 | 2 |
| 010401b | 010101c | 1 | 1 | 1 | 0 | 2 | 1 | 2 | 1 | 2 | 2 |
| 010401b | 010101d | 1 | 1 | 1 | 0 | 2 | 2 | 2 | 1 | 2 | 2 |
| 010401b | 010101e | 1 | 1 | 1 | 0 | 2 | 2 | 1 | 1 | 2 | 2 |
| 010401b | 010401b | 2 | 2 | 0 | 0 | 2 | 2 | 2 | 2 | 2 | 2 |
| 0105N | 010103 | 2 | 2 | 2 | 2 | 2 | 2 | 2 | 2 | 2 | 2 |
| 0105N | 010104 | 1 | 1 | 2 | 1 | 2 | 2 | 1 | 1 | 2 | 2 |
| 0105N | 010108 | 1 | 1 | 2 | 1 | 2 | 2 | 1 | 1 | 2 | 2 |
| 0105N | 010101a | 1 | 1 | 2 | 1 | 2 | 2 | 2 | 1 | 2 | 2 |
| 0105N | 010101b | 1 | 1 | 2 | 1 | 2 | 2 | 2 | 1 | 2 | 2 |
| 0105N | 010101c | 1 | 1 | 2 | 1 | 2 | 1 | 2 | 1 | 2 | 2 |
| 0105N | 010101d | 1 | 1 | 2 | 1 | 2 | 2 | 2 | 1 | 2 | 2 |
| 0105N | 010101e | 1 | 1 | 2 | 1 | 2 | 2 | 1 | 1 | 2 | 2 |
| 0105N | 010301a | 1 | 2 | 2 | 1 | 1 | 1 | 2 | 1 | 2 | 2 |
| 0105N | 010301b | 1 | 2 | 2 | 1 | 1 | 1 | 2 | 1 | 2 | 2 |
| 0105N | 010301c | 1 | 2 | 2 | 1 | 1 | 1 | 2 | 1 | 2 | 2 |
| 0105N | 010301d | 1 | 2 | 2 | 1 | 1 | 1 | 2 | 1 | 2 | 2 |

| Allele1 | Allele2 | −762T/C | −725C | −725G | −725T | −716G/T | −689G/A | −666T/G | −649A/G | −633A/G | −509C/G | −486C/A |
|---|---|---|---|---|---|---|---|---|---|---|---|---|
| 010301d | 010401a | 1 | 1 | 0 | 1 | 1 | 1 | 1 | 2 | 1 | 2 | 1 |
| 010301d | 010401b | 1 | 1 | 0 | 1 | 1 | 1 | 1 | 2 | 1 | 2 | 1 |
| 010401a | 010104 | 1 | 2 | 0 | 0 | 1 | 1 | 1 | 2 | 1 | 2 | 1 |
| 010401a | 010108 | 1 | 2 | 0 | 0 | 1 | 1 | 1 | 2 | 1 | 2 | 1 |
| 010401a | 010101a | 1 | 2 | 0 | 0 | 1 | 1 | 1 | 2 | 1 | 2 | 1 |
| 010401a | 010101b | 1 | 1 | 1 | 0 | 1 | 1 | 1 | 2 | 1 | 2 | 1 |
| 010401a | 010101c | 1 | 1 | 1 | 0 | 1 | 1 | 1 | 2 | 1 | 2 | 1 |
| 010401a | 010101d | 1 | 2 | 0 | 0 | 1 | 1 | 1 | 2 | 1 | 2 | 1 |
| 010401a | 010101e | 1 | 2 | 0 | 0 | 1 | 1 | 1 | 2 | 1 | 2 | 1 |
| 010401a | 010401a | 2 | 2 | 0 | 0 | 2 | 2 | 2 | 2 | 2 | 2 | 2 |
| 010401a | 010401b | 2 | 2 | 0 | 0 | 2 | 2 | 2 | 2 | 2 | 2 | 2 |
| 010401b | 010104 | 1 | 2 | 0 | 0 | 1 | 1 | 1 | 2 | 1 | 2 | 1 |
| 010401b | 010108 | 1 | 2 | 0 | 0 | 1 | 1 | 1 | 2 | 1 | 2 | 1 |
| 010401b | 010101a | 1 | 2 | 0 | 0 | 1 | 1 | 1 | 2 | 1 | 2 | 1 |
| 010401b | 010101b | 1 | 1 | 1 | 0 | 1 | 1 | 1 | 2 | 1 | 2 | 1 |
| 010401b | 010101c | 1 | 1 | 1 | 0 | 1 | 1 | 1 | 2 | 1 | 2 | 1 |
| 010401b | 010101d | 1 | 2 | 0 | 0 | 1 | 1 | 1 | 2 | 1 | 2 | 1 |
| 010401b | 010101e | 1 | 2 | 0 | 0 | 1 | 1 | 1 | 2 | 1 | 2 | 1 |
| 010401b | 010401b | 2 | 2 | 0 | 0 | 2 | 2 | 2 | 2 | 2 | 2 | 2 |
| 0105N | 010103 | 2 | 2 | 0 | 0 | 2 | 2 | 2 | 2 | 2 | 2 | 2 |
| 0105N | 010104 | 1 | 2 | 0 | 0 | 1 | 1 | 1 | 2 | 1 | 2 | 1 |
| 0105N | 010108 | 1 | 2 | 0 | 0 | 1 | 1 | 1 | 2 | 1 | 2 | 1 |
| 0105N | 010101a | 1 | 2 | 0 | 0 | 1 | 1 | 1 | 2 | 1 | 2 | 1 |
| 0105N | 010101b | 1 | 1 | 1 | 0 | 1 | 1 | 1 | 2 | 1 | 2 | 1 |
| 0105N | 010101c | 1 | 1 | 1 | 0 | 1 | 1 | 1 | 2 | 1 | 2 | 1 |
| 0105N | 010101d | 1 | 2 | 0 | 0 | 1 | 1 | 1 | 2 | 1 | 2 | 1 |
| 0105N | 010101e | 1 | 2 | 0 | 0 | 1 | 1 | 1 | 2 | 1 | 2 | 1 |
| 0105N | 010301a | 1 | 1 | 0 | 1 | 1 | 1 | 1 | 2 | 1 | 1 | 1 |
| 0105N | 010301b | 1 | 1 | 0 | 1 | 1 | 1 | 1 | 1 | 1 | 1 | 1 |
| 0105N | 010301c | 1 | 1 | 0 | 1 | 1 | 1 | 1 | 1 | 1 | 1 | 1 |
| 0105N | 010301d | 1 | 1 | 0 | 1 | 1 | 1 | 1 | 2 | 1 | 2 | 1 |

| Allele1 | Allele2 | −482A/G | −477G/C | −443G/A | −400G/A | −391G/A | −369A/G | −201A/G | −56C/T | +15A/G | +36A/G | AA31 (A/T) |
|---|---|---|---|---|---|---|---|---|---|---|---|---|
| 010301d | 010401a | 2 | 2 | 2 | 1 | 1 | 2 | 1 | 1 | 1 | 1 | 1 |
| 010301d | 010401b | 2 | 2 | 1 | 1 | 1 | 2 | 1 | 1 | 1 | 1 | 1 |
| 010401a | 010104 | 2 | 1 | 2 | 2 | 1 | 1 | 1 | 2 | 1 | 1 | 2 |
| 010401a | 010108 | 2 | 1 | 2 | 2 | 1 | 1 | 1 | 2 | 1 | 1 | 2 |
| 010401a | 010101a | 2 | 1 | 2 | 2 | 1 | 1 | 1 | 2 | 1 | 1 | 2 |
| 010401a | 010101b | 2 | 1 | 2 | 2 | 1 | 1 | 1 | 2 | 1 | 1 | 2 |
| 010401a | 010101c | 2 | 1 | 2 | 2 | 1 | 1 | 1 | 2 | 1 | 1 | 2 |
| 010401a | 010101d | 1 | 1 | 2 | 2 | 1 | 1 | 1 | 2 | 1 | 1 | 2 |
| 010401a | 010101e | 2 | 1 | 2 | 2 | 1 | 1 | 1 | 2 | 1 | 1 | 2 |
| 010401a | 010401a | 2 | 2 | 2 | 2 | 2 | 2 | 2 | 2 | 2 | 2 | 2 |
| 010401a | 010401b | 2 | 2 | 1 | 2 | 2 | 2 | 2 | 2 | 2 | 2 | 2 |
| 010401b | 010104 | 2 | 1 | 1 | 2 | 1 | 1 | 1 | 2 | 1 | 1 | 2 |
| 010401b | 010108 | 2 | 1 | 1 | 2 | 1 | 1 | 1 | 2 | 1 | 1 | 2 |
| 010401b | 010101a | 2 | 1 | 1 | 2 | 1 | 1 | 1 | 2 | 1 | 1 | 2 |
| 010401b | 010101b | 2 | 1 | 1 | 2 | 1 | 1 | 1 | 2 | 1 | 1 | 2 |
| 010401b | 010101c | 2 | 1 | 1 | 2 | 1 | 1 | 1 | 2 | 1 | 1 | 2 |
| 010401b | 010101d | 1 | 1 | 1 | 2 | 1 | 1 | 1 | 2 | 1 | 1 | 2 |
| 010401b | 010101e | 2 | 1 | 1 | 2 | 1 | 1 | 1 | 2 | 1 | 1 | 2 |
| 010401b | 010401b | 2 | 2 | 0 | 2 | 2 | 2 | 2 | 2 | 2 | 2 | 2 |
| 0105N | 010103 | 2 | 2 | 2 | 2 | 2 | 2 | 2 | 2 | 2 | 2 | 2 |
| 0105N | 010104 | 2 | 1 | 2 | 2 | 1 | 1 | 1 | 2 | 1 | 1 | 2 |
| 0105N | 010108 | 2 | 1 | 2 | 2 | 1 | 1 | 1 | 2 | 1 | 1 | 2 |
| 0105N | 010101a | 2 | 1 | 2 | 2 | 1 | 1 | 1 | 2 | 1 | 1 | 2 |
| 0105N | 010101b | 2 | 1 | 2 | 2 | 1 | 1 | 1 | 2 | 1 | 1 | 2 |
| 0105N | 010101c | 2 | 1 | 2 | 2 | 1 | 1 | 1 | 2 | 1 | 1 | 2 |
| 0105N | 010101d | 1 | 1 | 2 | 2 | 1 | 1 | 1 | 2 | 1 | 1 | 2 |
| 0105N | 010101e | 2 | 1 | 2 | 2 | 1 | 1 | 1 | 2 | 1 | 1 | 2 |
| 0105N | 010301a | 2 | 2 | 2 | 2 | 1 | 1 | 1 | 1 | 1 | 1 | 1 |
| 0105N | 010301b | 2 | 2 | 2 | 1 | 1 | 2 | 1 | 1 | 1 | 1 | 1 |
| 0105N | 010301c | 2 | 2 | 2 | 2 | 2 | 2 | 1 | 1 | 1 | 1 | 1 |
| 0105N | 010301d | 2 | 2 | 2 | 1 | 1 | 2 | 1 | 1 | 1 | 1 | 1 |

| | | AA35 | AA57 | AA69 | AA93 | AA100 | AA107 | AA110 | AA130 | | | |
|---|---|---|---|---|---|---|---|---|---|---|---|---|
| Allele1 | Allele2 | (A/T) | (A/G) | (C/T) | (T/C) | (C/T) | (A/T) | (C/A) | (C/D) | AA188 (C/T) | AA258 (C/T) | 14 bp in/del |
| 010301d | 010401a | 2 | 1 | 2 | 0 | 2 | 2 | 1 | 2 | 1 | 2 | 1 |
| 010301d | 010401b | 2 | 1 | 2 | 0 | 2 | 2 | 1 | 2 | 1 | 2 | 1 |
| 010401a | 010104 | 2 | 2 | 1 | 0 | 2 | 2 | 1 | 2 | 2 | 2 | 0 |
| 010401a | 010108 | 2 | 2 | 2 | 0 | 2 | 2 | 1 | 2 | 2 | 2 | 0 |
| 010401a | 010101a | 2 | 1 | 2 | 0 | 2 | 2 | 1 | 2 | 2 | 2 | 0 |
| 010401a | 010101b | 2 | 1 | 2 | 0 | 2 | 2 | 1 | 2 | 2 | 2 | 0 |
| 010401a | 010101c | 2 | 1 | 2 | 0 | 2 | 2 | 1 | 2 | 2 | 2 | 0 |
| 010401a | 010101d | 2 | 1 | 2 | 0 | 2 | 2 | 1 | 2 | 2 | 2 | 0 |
| 010401a | 010101e | 2 | 1 | 2 | 0 | 2 | 2 | 1 | 2 | 2 | 2 | 0 |
| 010401a | 010401a | 2 | 2 | 2 | 0 | 2 | 2 | 0 | 2 | 2 | 2 | 0 |
| 010401a | 010401b | 2 | 2 | 2 | 0 | 2 | 2 | 0 | 2 | 2 | 2 | 0 |
| 010401b | 010104 | 2 | 2 | 1 | 0 | 2 | 2 | 1 | 2 | 2 | 2 | 0 |
| 010401b | 010108 | 2 | 2 | 2 | 0 | 2 | 2 | 1 | 2 | 2 | 2 | 0 |
| 010401b | 010101a | 2 | 1 | 2 | 0 | 2 | 2 | 1 | 2 | 2 | 2 | 0 |
| 010401b | 010101b | 2 | 1 | 2 | 0 | 2 | 2 | 1 | 2 | 2 | 2 | 0 |
| 010401b | 010101c | 2 | 1 | 2 | 0 | 2 | 2 | 1 | 2 | 2 | 2 | 0 |
| 010401b | 010101d | 2 | 1 | 2 | 0 | 2 | 2 | 1 | 2 | 2 | 2 | 0 |
| 010401b | 010101e | 2 | 1 | 2 | 0 | 2 | 2 | 1 | 2 | 2 | 2 | 0 |
| 010401b | 010401b | 2 | 2 | 2 | 0 | 2 | 2 | 0 | 2 | 2 | 2 | 0 |
| 0105N | 010103 | 2 | 2 | 2 | 1 | 2 | 1 | 2 | 1 | 2 | 2 | 2 |
| 0105N | 010104 | 2 | 2 | 1 | 1 | 2 | 2 | 2 | 1 | 2 | 2 | 1 |
| 0105N | 010108 | 2 | 2 | 2 | 1 | 2 | 2 | 2 | 1 | 2 | 2 | 1 |
| 0105N | 010101a | 2 | 1 | 2 | 1 | 2 | 2 | 2 | 1 | 2 | 2 | 1 |
| 0105N | 010101b | 2 | 1 | 2 | 1 | 2 | 2 | 2 | 1 | 2 | 2 | 1 |
| 0105N | 010101c | 2 | 1 | 2 | 1 | 2 | 2 | 2 | 1 | 2 | 2 | 1 |
| 0105N | 010101d | 2 | 1 | 2 | 1 | 2 | 2 | 2 | 1 | 2 | 2 | 1 |
| 0105N | 010101e | 2 | 1 | 2 | 1 | 2 | 2 | 2 | 1 | 2 | 2 | 1 |
| 0105N | 010301a | 2 | 1 | 2 | 1 | 2 | 2 | 2 | 1 | 1 | 2 | 2 |
| 0105N | 010301b | 2 | 1 | 2 | 1 | 2 | 2 | 2 | 1 | 1 | 2 | 2 |
| 0105N | 010301c | 2 | 1 | 2 | 1 | 2 | 2 | 2 | 1 | 1 | 2 | 2 |
| 0105N | 010301d | 2 | 1 | 2 | 1 | 2 | 2 | 2 | 1 | 1 | 2 | 2 |

| Allele1 | Allele2 | −1306A/G | −1179G/A | −1155G/A | −1140T/A | −1138A/G | −1121C/T | −990G/A | −0964A/G | −922C/A | −810C/T |
|---|---|---|---|---|---|---|---|---|---|---|---|
| 0105N | 010401a | 2 | 2 | 1 | 1 | 2 | 2 | 2 | 2 | 2 | 2 |
| 0105N | 010401b | 2 | 2 | 1 | 1 | 2 | 2 | 2 | 2 | 2 | 2 |
| 0105N | 0105N | 2 | 2 | 2 | 2 | 2 | 2 | 2 | 2 | 2 | 2 |

| Allele1 | Allele2 | −762T/C | −725C | −725G | −725T | −716G/T | −689G/A | −666T/G | −649A/G | −633A/G | −509C/G | −486C/A |
|---|---|---|---|---|---|---|---|---|---|---|---|---|
| 0105N | 010401a | 2 | 2 | 0 | 0 | 2 | 2 | 2 | 2 | 2 | 2 | 2 |
| 0105N | 010401b | 2 | 2 | 0 | 0 | 2 | 2 | 2 | 2 | 2 | 2 | 2 |
| 0105N | 0105N | 2 | 2 | 0 | 0 | 2 | 2 | 2 | 2 | 2 | 2 | 2 |

| Allele1 | Allele2 | −482A/G | −477G/C | −443G/A | −400G/A | −391G/A | −369A/G | −201A/G | −56C/T | +15A/G | +36A/G | AA31 (A/T) |
|---|---|---|---|---|---|---|---|---|---|---|---|---|
| 0105N | 010401a | 2 | 2 | 2 | 2 | 2 | 2 | 2 | 2 | 2 | 2 | 2 |
| 0105N | 010401b | 2 | 2 | 1 | 2 | 2 | 2 | 2 | 2 | 2 | 2 | 2 |
| 0105N | 0105N | 2 | 2 | 2 | 2 | 2 | 2 | 2 | 2 | 2 | 2 | 2 |
| 010103 | 01010g | | | | | | | | | 2 | 2 | 2 |
| 010103 | G3d5 | | | | | | | | | 2 | 2 | 2 |
| 010601 | 01010g | | | | | | | | | | 2 | 2 |
| 010601 | G3d5 | | | | | | | | | | 2 | 2 |
| 010102a | 01010g | | | | | | | | | 2 | 2 | 2 |
| 010102a | G3d5 | | | | | | | | | 2 | 2 | 2 |
| 010102b | 01010g | | | | | | | | | 2 | 2 | 2 |
| 010102b | G3d5 | | | | | | | | | 2 | 2 | 2 |
| 010102c | 01010g | | | | | | | | | 2 | 2 | 2 |
| 010102c | G3d5 | | | | | | | | | 2 | 2 | 2 |
| 01010g | 010104 | | | | | | | | | 1 | 1 | 2 |
| 01010g | 010108 | | | | | | | | | 1 | 1 | 2 |
| 01010g | 010101a | | | | | | | | | 1 | 1 | 2 |
| 01010g | 010101b | | | | | | | | | 1 | 1 | 2 |
| 01010g | 010101c | | | | | | | | | 1 | 1 | 2 |
| 01010g | 010101d | | | | | | | | | 1 | 1 | 2 |
| 01010g | 010101e | | | | | | | | | 1 | 1 | 2 |
| 01010g | 01010g | | | | | | | | | 2 | 2 | 2 |
| 01010g | 010301a | | | | | | | | | 1 | 1 | 1 |
| 01010g | 010301b | | | | | | | | | 1 | 1 | 1 |
| 01010g | 010301c | | | | | | | | | 1 | 1 | 1 |
| 01010g | 010301d | | | | | | | | | 1 | 1 | 1 |
| 01010g | 010401a | | | | | | | | | 2 | 2 | 2 |
| 01010g | 010401b | | | | | | | | | 2 | 2 | 2 |
| 01010g | G3d5 | | | | | | | | | 2 | 2 | 2 |
| 0105N | 01010g | | | | | | | | | 2 | 2 | 2 |
| 0105N | G3d5 | | | | | | | | | 2 | 2 | 2 |

| | | AA35 | AA57 | AA69 | AA93 | AA100 | AA107 | AA110 | AA130 | | | |
|---|---|---|---|---|---|---|---|---|---|---|---|---|
| Allele1 | Allele2 | (A/T) | (A/G) | (C/T) | (T/C) | (C/T) | (A/T) | (C/A) | (C/D) | AA188 (C/T) | AA258 (C/T) | 14 bp in/del |
| 0105N | 010401a | 2 | 2 | 2 | 1 | 2 | 2 | 1 | 1 | 2 | 2 | 1 |
| 0105N | 010401b | 2 | 2 | 2 | 1 | 2 | 2 | 1 | 1 | 2 | 2 | 1 |
| 0105N | 0105N | 2 | 2 | 2 | 2 | 2 | 2 | 2 | 0 | 2 | 2 | 2 |
| 010103 | 01010g | 1 | 2 | 2 | 1 | 2 | 1 | 2 | 2 | 2 | 2 | 2 |
| 010103 | G3d5 | 2 | 2 | 2 | 1 | 1 | 1 | 2 | 2 | 2 | 2 | 2 |
| 010601 | 01010g | 1 | 2 | 2 | 2 | 2 | 2 | 2 | 2 | 2 | 1 | 2 |
| 010601 | G3d5 | 2 | 2 | 2 | 2 | 1 | 2 | 2 | 2 | 2 | 1 | 2 |
| 010102a | 01010g | 1 | 2 | 2 | 2 | 2 | 2 | 2 | 2 | 2 | 2 | 2 |
| 010102a | G3d5 | 2 | 2 | 2 | 2 | 1 | 2 | 2 | 2 | 2 | 2 | 2 |
| 010102b | 01010g | 1 | 2 | 2 | 2 | 2 | 2 | 2 | 2 | 2 | 2 | 2 |
| 010102b | G3d5 | 2 | 2 | 2 | 2 | 1 | 2 | 2 | 2 | 2 | 2 | 2 |
| 010102c | 01010g | 1 | 2 | 2 | 2 | 2 | 2 | 2 | 2 | 2 | 2 | 2 |
| 010102c | G3d5 | 2 | 2 | 2 | 2 | 1 | 2 | 2 | 2 | 2 | 2 | 2 |
| 01010g | 010104 | 1 | 2 | 1 | 1 | 2 | 2 | 2 | 2 | 2 | 2 | 1 |
| 01010g | 010108 | 1 | 2 | 2 | 1 | 2 | 2 | 2 | 2 | 2 | 2 | 1 |
| 01010g | 010101a | 1 | 1 | 2 | 1 | 2 | 2 | 2 | 2 | 2 | 2 | 1 |
| 01010g | 010101b | 1 | 1 | 2 | 1 | 2 | 2 | 2 | 2 | 2 | 2 | 1 |
| 01010g | 010101c | 1 | 1 | 2 | 1 | 2 | 2 | 2 | 2 | 2 | 2 | 1 |
| 01010g | 010101d | 1 | 1 | 2 | 1 | 2 | 2 | 2 | 2 | 2 | 2 | 1 |
| 01010g | 010101e | 1 | 1 | 2 | 1 | 2 | 2 | 2 | 2 | 2 | 2 | 1 |
| 01010g | 01010g | 0 | 2 | 2 | 2 | 2 | 2 | 2 | 2 | 2 | 2 | 2 |
| 01010g | 010301a | 1 | 1 | 2 | 1 | 2 | 2 | 2 | 2 | 1 | 2 | 2 |
| 01010g | 010301b | 1 | 1 | 2 | 1 | 2 | 2 | 2 | 2 | 1 | 2 | 2 |
| 01010g | 010301c | 1 | 1 | 2 | 1 | 2 | 2 | 2 | 2 | 1 | 2 | 2 |
| 01010g | 010301d | 1 | 1 | 2 | 1 | 2 | 2 | 2 | 2 | 1 | 2 | 2 |
| 01010g | 010401a | 1 | 2 | 2 | 1 | 2 | 2 | 1 | 2 | 2 | 2 | 1 |
| 01010g | 010401b | 1 | 2 | 2 | 1 | 2 | 2 | 1 | 2 | 2 | 2 | 1 |
| 01010g | G3d5 | 1 | 2 | 2 | 2 | 1 | 2 | 2 | 2 | 2 | 2 | 2 |
| 0105N | 01010g | 1 | 2 | 2 | 2 | 2 | 2 | 2 | 1 | 2 | 2 | 2 |
| 0105N | G3d5 | 2 | 2 | 2 | 2 | 1 | 2 | 2 | 1 | 2 | 2 | 2 |

| | | | | | | | | | | | |
|---|---|---|---|---|---|---|---|---|---|---|---|
| Allele1 | Allele2 | −1306A/G | −1179G/A | −1155G/A | −1140T/A | −1138A/G | −1121C/T | −990G/A | −0964A/G | −922C/A | −810C/T |

| | | | | | | | | | | | | |
|---|---|---|---|---|---|---|---|---|---|---|---|---|
| Allele1 | Allele2 | −762T/C | −725C | −725G | −725T | −716G/T | −689G/A | −666T/G | −649A/G | −633A/G | −509C/G | −486C/A |

| Allele1 | Allele2 | −482A/G | −477G/C | −443G/A | −400G/A | −391G/A | −369A/G | −201A/G | −56C/T | +15A/G | +36A/G | AA31 (A/T) |
|---|---|---|---|---|---|---|---|---|---|---|---|---|
| G3d5 | 010104 | | | | | | | | | 1 | 1 | 2 |
| G3d5 | 010108 | | | | | | | | | 1 | 1 | 2 |
| G3d5 | 010101a | | | | | | | | | 1 | 1 | 2 |
| G3d5 | 010101b | | | | | | | | | 1 | 1 | 2 |
| G3d5 | 010101c | | | | | | | | | 1 | 1 | 2 |
| G3d5 | 010101d | | | | | | | | | 1 | 1 | 2 |
| G3d5 | 010101e | | | | | | | | | 1 | 1 | 2 |
| G3d5 | 010301a | | | | | | | | | 1 | 1 | 1 |
| G3d5 | 010301b | | | | | | | | | 1 | 1 | 1 |
| G3d5 | 010301c | | | | | | | | | 1 | 1 | 1 |
| G3d5 | 010301d | | | | | | | | | 1 | 1 | 1 |
| G3d5 | 010401a | | | | | | | | | 2 | 2 | 2 |
| G3d5 | 010401b | | | | | | | | | 2 | 2 | 2 |
| G3d5 | G3d5 | | | | | | | | | 2 | 2 | 2 |

| | | AA35 | AA57 | AA69 | AA93 | AA100 | AA107 | AA110 | AA130 | | | |
|---|---|---|---|---|---|---|---|---|---|---|---|---|
| Allele1 | Allele2 | (A/T) | (A/G) | (C/T) | (T/C) | (C/T) | (A/T) | (C/A) | (C/D) | AA188 (C/T) | AA258 (C/T) | 14 bp in/del |
| G3d5 | 010104 | 2 | 2 | 1 | 1 | 1 | 2 | 2 | 2 | 2 | 2 | 1 |
| G3d5 | 010108 | 2 | 2 | 2 | 1 | 1 | 2 | 2 | 2 | 2 | 2 | 1 |
| G3d5 | 010101a | 2 | 1 | 2 | 1 | 1 | 2 | 2 | 2 | 2 | 2 | 1 |
| G3d5 | 010101b | 2 | 1 | 2 | 1 | 1 | 2 | 2 | 2 | 2 | 2 | 1 |
| G3d5 | 010101c | 2 | 1 | 2 | 1 | 1 | 2 | 2 | 2 | 2 | 2 | 1 |
| G3d5 | 010101d | 2 | 1 | 2 | 1 | 1 | 2 | 2 | 2 | 2 | 2 | 1 |
| G3d5 | 010101e | 2 | 1 | 2 | 1 | 1 | 2 | 2 | 2 | 2 | 2 | 1 |
| G3d5 | 010301a | 2 | 1 | 2 | 1 | 1 | 2 | 2 | 2 | 1 | 2 | 2 |
| G3d5 | 010301b | 2 | 1 | 2 | 1 | 1 | 2 | 2 | 2 | 1 | 2 | 2 |
| G3d5 | 010301c | 2 | 1 | 2 | 1 | 1 | 2 | 2 | 2 | 1 | 2 | 2 |
| G3d5 | 010301d | 2 | 1 | 2 | 1 | 1 | 2 | 2 | 2 | 1 | 2 | 2 |
| G3d5 | 010401a | 2 | 2 | 2 | 1 | 1 | 2 | 1 | 2 | 2 | 2 | 1 |
| G3d5 | 010401b | 2 | 2 | 2 | 1 | 1 | 2 | 1 | 2 | 2 | 2 | 1 |
| G3d5 | G3d5 | 2 | 2 | 2 | 2 | 0 | 2 | 2 | 2 | 2 | 2 | 2 |

### Publications Cited

The publications cited below are incorporated by reference to the extent they teach a material or method disclosed herein:
Ober et al. (2003) Am. J. Hum. Genet. 72: 1425-1435.
Aldrich C L, et al. (2001) Molec Hum Reprod 7:1162-1172
Hoffjan S, Ober C (2002) Curr Opin Immunol 14:709-717
Hviid T V, et al. (1999) Hum Immun 60:1237-1244
Kovats S, et al. (1990) Science 248:220-223
Mathew C G, Lewis C M (2004) Hum Mol Genet 13 Suppl 1:R161-168
Ober C (1998) Amer J Hum Genet 62:1-5
Ober C, et al. (1998) Placenta 19:127-132
Ober C, et al. (2000) Am J Hum Genet 67:1154-1162
Pfeiffer K A, Fimmers R, Engels G, van Der Ven H, van Der Ven K (2001) Mol Hum Reprod 7:373-378
Soderhall C, et al. (2001) Hum Genet 109:129-135
Wright P, et al. (2001) Schizophr Res 47:1-12
Xu J, et al. (2001) Am J Hum Genet 68:1437-1446.
Zhang X J, et al. (2002) J Invest Dermatol 119:1361-1366
Aldrich C, et al. (2000) J Reprod Immunol 47:41-48
Aldrich C, et al. (2002) Hum Immunol 63:405-412
Cavalcanti A, et al. (2002) Molecular Biology & Evolution 19:256-262
Christiansen O B, et al. (1989) Tissue Antigens 34:190-199
Diggle P J, et al. (1994) Analysis of Longitudinal Data. Oxford University Press, New York
Frommer M, et al. (1992) Proc Natl Acad Sci USA 89:1827-1831
Hauck W W, et al. (1991) Genet Epid 8:187-197
Hostetler J A (1974) Hutterite Society. Johns Hopkins University Press, Baltimore
Kostyu D D, et al. (1989) Am J Hum Genet 45:261-269
Lefebvre S, et al. (1999) Journal of Reproductive Immunology 43:213-24
Martin A O (1970) Am J Phys Anthropology 32:351-368
Moreau P, et al. (2003) Proc Natl Acad Sci USA 100:1191-1196
Ober C (1997) J Reprod Immunol 33:89-90
Ober C, et al. (1992) Amer J Hum Genet 50:6-14
Ober C, et al. (1998b) Hum Reprod 13:33-38
Ober C, et al. (1999) Am J Human Genet 64:225-231
Ober C, et al. (1996a) J Reprod Immunol 32:111-23
Ober C, et al. (1996b) J Reprod Immunol 32:111-123
Olek A, et al. (1996) Nucleic Acids Res 24:5064-5066
Schmidt C M, et al. (1993) Crit Rev Immunol 13:207-24
Schmidt C M, et al. (1995) Immunology Reviews 147:53-65
Sheps M C (1965) Popul Stud 19:65-80
Steinberg A G, et al. (1967) Johns Hopkins University Press, Baltimore, pp 267-290
Stephenson M D, et al. (2002) Human Reproduction 17:446-451
Suarez M B, et al. (1997) Immunogenetics 45:464-465
Warburton D, et al. (1964) Hum Genet 16:1-25
Warnecke P M, et al. (2002) Methods 27:101-107
Weitkamp L R, et al. (1999) Immunogenetics 49:491-497
Zangenberg G, et al. (1995) Nat Genet 10:407-14.
Bourgain, C. et al. Am J Hum Genet 73, 612-626 (2003).
Gudbjartsson, D. F., Jonasson, K., Frigge, M. L. & Kong, A. Nat Genet 25, 12-13 (2000).
Kong, A. & Cox, N. J. Am J Hum Genet 61, 1179-1188 (1997).
Lester, L. A. et al. J Allergy Clin Immunol 108, 357-362 (2001).
Morales, P. J. et al. J Immunol 171, 6215-6224 (2003).
Ober, C. et al. Am J Hum Genet 72, 1425-1435 (2003).
Panhuysen, C. I., Bleecker, E. R., Koeter, G. H., Meyers, D. A. & Postma, D. S. Clin Exp 1995
Spielman, R. S., McGinnis, R. E. & Ewens, W. J. Amer J Hum Genet 52, 506-516 (1993).

1. A method of genotyping a biological sample for HLA-G, the method comprising:
(a) identifying a plurality of SNPs in the HLA-G region; and
(b) translating information on the SNPs into a genotype.

2. The method of claim 1, wherein the plurality of SNPs are located in the promoter region of HLA-G.

3. The method of claim 2, further comprising a plurality of SNPs located in the exons of HLA-G.

4. The method of claim 2, wherein the SNPs are listed in Table 7.

5. The method of claim 2, wherein the SNPs are identified by direct sequencing.

6. The method of claim 3, wherein the SNPs are listed in Table 8.

7. The method of claim 3, wherein the SNPs are identified by hybridization with specific probes.

8. The method of claim 2 wherein the promoter region of HLA-G comprises a DNA sequence from nucleotide position about 1350 to about +50 of the HLA-G promoter.

9. The method of claim 5, wherein the HLA-G promoter region is amplified by oligonucleotides selected from the group consisting of nucleotide sequences designated 5′-MCAGTGCTAGAGCCACAG (SEQ ID NO: 1), 5′-AACAGTGCTAGAGCCACAA (SEQ ID NO: 2), 5′-GMGAGGGTTCGGGGC (SEQ ID NO: 3), and 5′-GAAGAGGGTTCGGGGT (SEQ ID NO: 4).

10. The method of claim 1 wherein the genotype comprises a string of pseudo codes consisting of 0, 1 and 2.

11. The method of claim 10, wherein the string of pseudo codes is selected from the string of pseudo codes listed in Table 8.

12. A method for predicting an immune-associated risk in a subject, the method comprising:
(a) obtaining an HLA-G genotype of the subject; and
(b) determining the immune-related risk of the subject by comparing the subject's HLA-G genotype with a reference HLA-G genotype.

13. The method of claim 12, wherein the immune-associated risk is selected from the group consisting of asthma, allergy, auto-immune disorder, infertility, fetal miscarriage, and tissue or organ transplantation.

14. The method of claim 12, wherein the immune-associated risk is for a Th2-skewed immunologic condition.

15. An assay for a candidate drug to ameliorate a symptom of an immunologic condition, the assay comprising:
(a) determining association of HLA-G expression with the immunologic condition; and
(b) determining if the candidate drug affects the expression of the associated HLA-G.

16. A method for ameliorating the effects of an immunologic condition, the method comprising:
(a) determining an HLA-G polymorphism that is associated with the condition;
(b) determining an effect of the polymorphism on a metabolic pathway; and
(c) modulating the effect to ameliorate the immunologic condition.

17. Use of an HLA-G asthma susceptibility gene to develop an inhibitor of an asthma symptom in a person at risk and to deliver the inhibitor to the person.

18. The use of claim 17, wherein the inhibitor is delivered by an inhaler.

19. A diagnostic kit for HLA-G associated conditions, the kit comprising:
(a) primers capable of amplifying HLA-G; and
(b) reagents to amplify HLA-G and detect SNPs.

20. The diagnostic kit of claim 19, wherein primers are specific for promoter haplotypes and reagents are for sequencing to detect SNPs.

21. The diagnostic kit of claim 19, wherein primers are specific for all SNP polymorphisms and reagents are for hybridization to detect SNPs.

22. The diagnostic kit of claim 19, further comprising information on HLA-G genotype associated risks for immunologic conditions.
